(19) United States
(12) Patent Application Publication
(10) Pub. No.: US 2024/0127905 A1
(43) Pub. Date: Apr. 18, 2024

(54) INTEGRATING VARIANT CALLS FROM MULTIPLE SEQUENCING PIPELINES UTILIZING A MACHINE LEARNING ARCHITECTURE

(71) Applicant: Illumina, Inc., San Diego, CA (US)

(72) Inventors: Gavin Derek Parnaby, Laguna Niguel, CA (US); Seyedmohammadjafar Hashemidoulabi, Lakewood, CO (US); Aaron L. Halpern, San Carlos, CA (US); Michael Ruehle, Fort Worth, TX (US)

(21) Appl. No.: 18/481,038

(22) Filed: Oct. 4, 2023

Related U.S. Application Data
(60) Provisional application No. 63/482,163, filed on Jan. 30, 2023.
(60) Provisional application No. 63/378,474, filed on Oct. 5, 2022.

(51) Int. Cl. G16B 20/20

(52) U.S. Cl. CPC G16B 20/20

(57) ABSTRACT

This disclosure describes methods, non-transitory computer readable media, and systems that can generate genotype calls from a combined pipeline for processing nucleotide reads from multiple read types/sources for robust, accurate genotype calls. For example, the disclosed systems can train and/or utilize a genotype-call-integration machine-learning model to generate predictions for genotype calls based on data associated with a first type of nucleotide reads (e.g., short reads) and a second type of nucleotide reads (e.g., long reads). As disclosed, the disclosed systems can determine sequencing metrics and can utilize a genotype-call-integration machine-learning model to generate predictions (e.g., genotype probabilities, variant call classifications) for generating output genotype calls based on the sequencing metrics. The disclosed system can utilize multiple such genotype-call-integration machine-learning models to generate genotype calls for different variant types, such as SNPs and indels, where the genotype-call-integration machine-learning models generate different predictions for each variant type.

### CROSS-REFERENCE TO RELATED APPLICATIONS

The present application claims the benefit of, and priority to, U.S. Provisional Application No. 63/482,163, entitled “INTEGRATING VARIANT CALLS FROM MULTIPLE SEQUENCING PIPELINES UTILIZING A MACHINE LEARNING ARCHITECTURE,” filed on Jan. 30, 2023; and U.S. Provisional Application No. 63/378,474, entitled “INTEGRATING VARIANT CALLS FROM MULTIPLE SEQUENCING PIPELINES UTILIZING A MACHINE LEARNING ARCHITECTURE,” filed on Oct. 5, 2022. The aforementioned applications are hereby incorporated by reference in their entirety.

### BACKGROUND

In recent years, biotechnology firms and research institutions have improved hardware and software for sequencing nucleotides and determining nucleotide base calls for reads and, subsequently, variant and genotype calls for genomic samples. For instance, some existing nucleobase sequencing platforms determine individual nucleotide bases (or “nucleobases”) within sequences by using conventional Sanger sequencing or by using sequencing-by-synthesis (SBS) methods. When using SBS, existing platforms can monitor many thousands of nucleic acid polymers being synthesized in parallel to predict genotype calls from a larger base call dataset. For instance, a camera in many SBS platforms captures images of irradiated fluorescent tags incorporated into oligonucleotides for determining the nucleobase calls. After capturing such images, existing SBS platforms send base call data (or image data) to a computing device to apply sequencing data analysis software that determines a nucleobase sequence for a nucleic acid polymer. Based on differences between the aligned nucleotide reads and the reference genome, existing systems can further utilize a variant caller to identify variants of a genomic sample, such as single nucleotide polymorphisms (SNPs), insertions and deletions (indels), and/or structural variants, and genotype calls.

Despite these recent advances in sequencing and variant calling, existing sequencing systems often include variant callers that inaccurately determine variant calls, especially for SNPs and indels. For example, many existing systems generate variant calls that include excessive numbers of false positive calls and/or false negative calls for SNPs and indels. Contributing to this inaccuracy, the constraints of some existing sequencing systems dictate that they generate variant calls from single-stream processing pipelines that focus on one read source at a time. For instance, as suggested above, some existing systems perform variant calling and/or variant call filtering based solely on nucleotide reads from SBS sequencing. As a further example, some existing systems perform variant calling based solely on nucleotide reads from certain types of long reads, such as circular consensus sequencing (CCS) reads or nanopore long reads. Consequently, relying exclusively on single sources for read data results in many existing systems generating variant calls that include excessive numbers of false positive calls and/or false negative calls for certain clinical benchmarks that could otherwise be reduced with a more accurate system. To further compound the problem, different sequencing systems exhibit different error profiles, such as when prior systems generate variant calls with higher indel errors based on CCS reads and nanopore long reads relative to sequencing systems using other types of reads.

To compound such variant calling inaccuracy, some existing sequencing systems utilize models that require training on millions or billions of base call data that are either unavailable or incomplete. More specifically, some existing sequencing systems utilize deep learning models that require an excessive amount of training data to achieve acceptable measures of accuracy. However, training data for variants is relatively limited for certain variant types (e.g., structural variants), and training models using incomplete or insubstantial data results in inaccurate and unreliable variant call predictions. Thus, some existing systems that rely on deep learning models can produce inaccurate variant calls, including SNPs and indels.

In addition to inaccurately determining variant calls, some existing sequencing systems also inefficiently expend computing resources with overly complex models. Specifically, the variant callers of some existing sequencing systems are computationally expensive and slow. Indeed, some existing sequencing systems utilize variant callers with deep learning architectures that require extensive computational resources (e.g., computing time, processing power, and memory) to train and apply the deep learning architectures. For example, some existing sequencing systems consume hundreds of hours and multiple graphical processing units (GPUs) to train complex convolutional neural networks or other deep learning architectures that, even after training, consume many hours (e.g., up to 24 hours) across multiple computing devices to generate variant calls or genotype calls for a single sample sequence.

As an added drawback of existing sequencing systems with complex deep learning networks, many such systems utilize model architectures that render sequence data uninterpretable. More specifically, as the basis for generating a variant call, some existing deep neural networks transform and manipulate sequence data many times over, changing from one uninterpretable latent vector to another such latent vector across the various layers and neurons during processing. In many cases, the internal data of these deep neural networks is uninterpretable and difficult to utilize in any way outside of the neural network architecture itself.

### SUMMARY

This disclosure describes embodiments of methods, non-transitory computer readable media, and systems that can utilize a machine learning model to generate predictions for genotype calls based on data from different types of nucleotide reads. In particular, the disclosed systems can generate genotype calls from a combined pipeline for processing nucleotide reads from multiple read types/sources for robust, accurate genotype calls (including constituent variant calls). For example, the disclosed systems can train or utilize a genotype-call-integration machine-learning model to generate predictions for genotype calls based on data associated with a first type of nucleotide reads (e.g., short reads) and a second type of nucleotide reads (e.g., long reads). As disclosed, the systems can determine sequencing metrics for a first genotype call corresponding to a first type of nucleotide reads and a second genotype call corresponding to a second type of nucleotide reads. Based on different or shared sequencing metrics corresponding to first and second genotype calls, the disclosed systems utilize a genotype-call-integration machine-learning model to generate predictions (e.g., genotype probabilities, variant call classifications) for updating or confirming the first genotype call or the second genotype call, or determining a different genotype call. In some cases, the disclosed system can utilize multiple such genotype-call-integration machine-learning models to update or confirm genotype calls for different variant types, such as SNPs and indels, where the genotype-call-integration machine-learning models generate different predictions for each variant type.

### BRIEF DESCRIPTION OF THE DRAWINGS

The detailed description refers to the drawings briefly described below.

FIG. 1 illustrates a block diagram of an example computing environment for implementing a sequencing system and a call integration system in accordance with one or more embodiments.

FIG. 2 illustrates an overview of the call integration system generating a genotype call utilizing a genotype-call-integration machine-learning model in accordance with one or more embodiments.

FIG. 3 illustrates example types of nucleotide reads based upon which the call integration system can generate genotype calls in accordance with one or more embodiments.

FIGS. 4A-4C illustrate the call integration system determining sequencing metrics shared or differing among different types of nucleotide reads in accordance with one or more embodiments.

FIGS. 5A-5C illustrate the call integration system generating predictions (e.g., genotype probabilities or variant call classifications) and corresponding genotype calls utilizing a genotype-call-integration machine-learning model in accordance with one or more embodiments.

FIG. 6 illustrates an example diagram of a training process for learning parameters of the genotype-call-integration machine-learning model in accordance with one or more embodiments.

FIG. 7 illustrates an example diagram for updating or generating a merged variant call file based on predictions of a genotype-call-integration machine-learning model in accordance with one or more embodiments.

FIG. 8 illustrates example graphs and tables of accuracy metrics for the call integration system in accordance with one or more embodiments.

FIG. 9A illustrates example tables of accuracy metrics for the call integration system in accordance with one or more embodiments.

FIG. 9B illustrates an example table of accuracy metrics for the call integration system in accordance with one or more embodiments.

FIGS. 10A-10B illustrate graphs depicting accuracy metrics associated with the call integration system in accordance with one or more embodiments.

FIG. 11 illustrates a flowchart of a series of acts for generating a genotype call from nucleotide reads of a first read type and a second read type utilizing a genotype-call-integration machine-learning model in accordance with one or more embodiments.

FIG. 12 illustrates a block diagram of an example computing device for implementing one or more embodiments of the present disclosure.

### DETAILED DESCRIPTION

This disclosure describes embodiments of a call integration system that generates and modifies genotype calls for a genomic sample utilizing a genotype-call-integration machine-learning model. In particular, the call integration system can utilize a genotype-call-integration machine-learning model to generate an output genotype call (e.g., a reported genotype call from a merged variant call file) from multiple initial genotype calls (e.g., variant calls) for a genomic locus generated by a call generation model from different read types. To generate an output genotype call, in certain embodiments, the call integration system generates or receives initial genotype calls from read data associated with a combination of short reads (e.g., sequencing-by-synthesis or “SBS” reads) and long reads (e.g., nanopore long reads, circular consensus sequencing or “CCS” reads, and/or assembled nucleotide reads). In some cases, the call integration system determines or identifies specific sequencing metrics (e.g., from read data, call generation model data, and/or external data) to input into the genotype-call-integration machine-learning model for generating an output genotype call. The call integration system can further train or apply the genotype-call-integration machine-learning model according to the sequencing metrics to generate (or refine or recalibrate) genotype calls.

As just mentioned, in certain implementations, the call integration system improves genotype calling accuracy (and corresponding variant calling accuracy) using read data from different read types. To facilitate generating genotype calls from multiple read types, in some embodiments, the call integration system receives initial genotype calls from a call generation model. For instance, the call integration system (i) receives or determines an initial genotype call (e.g., a call indicating a genotype at a genomic coordinate of a nucleotide sequence) corresponding to a first type of nucleotide reads (e.g., short reads) and further (ii) receives or determines another initial genotype call corresponding to a second type of nucleotide reads (e.g., long reads). In some cases, the first type of nucleotide reads includes nucleotide reads synthesized from sample library fragments that are shorter than the first threshold number of nucleobases. Conversely, in the same or other cases, the second type of nucleotide reads includes (i) assembled nucleotide reads that have been assembled from shorter nucleotide reads to form a contiguous sequence satisfying a second threshold number of nucleobases, (ii) CCS reads satisfying the second threshold number of nucleobases, and/or (iii) nanopore long reads satisfying the second threshold number of nucleobases.

From the initial genotype calls corresponding to the different read types, the call integration system can further generate an output genotype call, such as a prediction of the presence or absence of a variant, such as an SNP or an indel, and zygosity of a genomic sample's alleles. As mentioned, to generate the output genotype call, the call integration system can extract, identify, or determine sequencing metrics (associated with initial genotype calls from the different read types) to input into a genotype-call-integration machine-learning model. In turn, the genotype-call-integration machine-learning model generates a set of likelihoods or predictions (e.g., a different set of predictions for each initial genotype call corresponding to the different read types and/or for each different variant type) that indicate likelihoods that the initial genotype calls are correct or incorrect. For instance, the call integration system can extract or determine sequencing metrics belonging to one or more categories, including: (i) read-based sequencing metrics, (ii) call-model-generated sequencing metrics, and (iii) externally sourced sequencing metrics. Additional detail regarding the makeup and determination of sequencing metrics is provided below with reference to the figures.

As suggested, in certain embodiments, the call integration system generates genotype calls using a multi-stream pipeline that processes multiple read types for an output genotype call as part of a combined or merged variant call file based on the multiple read types. For instance, the call integration system (i) processes a first set of sequencing metrics extracted from an initial genotype call based on a first read type and (ii) processes a second set of sequencing metrics extracted from an initial genotype call based on a second read type. In addition, the call integration system can utilize a genotype-call-integration machine-learning model to generate a set of predictions based on the first and second sets of sequencing metrics and can generate an output genotype call from the set of predictions.

To predict or generate output genotype calls for different variant types (e.g., SNPs and indels), in some cases, the call integration system generates different sets of predictions for the different variant types (e.g., from the same or different sequencing metrics) utilizing the genotype-call-integration machine-learning model. For example, the call integration system can utilize a first instance of a genotype-call-integration machine-learning model (e.g., trained to predict SNPs) to process first-read-type sequencing metrics (e.g., SBS sequencing metrics) and second-read-type sequencing metrics (e.g., assembled-nucleotide-read sequencing metrics) to generate an output genotype call for an SNP at a genomic coordinate. In addition, the call integration system can utilize a second genotype-call-integration machine-learning model (e.g., trained to predict indels) to process first-read-type sequencing metrics and second-read-type sequencing metrics to generate an output genotype call for an indel at a different (or the same) genomic coordinate. In some embodiments, the call integration system can utilize a first genotype-call-integration machine-learning model for biallelic SNPs and can utilize a second genotype-call-integration machine-learning model for variant calls of other types (e.g., variants that are not biallelic SNPs). Further, while this disclosure describes at least two different types of genotype-call-integration machine-learning models, in certain implementations, the call integration system trains or applies a single genotype-call-integration machine-learning model to generate both genotype predictions for different types of variants (e.g., genotype predictions for either SNPs or indels).

As suggested above, the call integration system provides several advantages, benefits, and/or improvements over existing sequencing systems, including variant callers and other sequencing data analysis software. For instance, the call integration system generates more accurate genotype calls (including variant calls) than existing sequencing systems. While some prior sequencing systems inaccurately generate variant calls (especially for SNPs and indels), the call integration system trains or utilizes a genotype-call-integration machine-learning model to improve genotype/variant calling over prior systems. Specifically, unlike prior systems that rely on single sources for read data, the call integration system can process multiple reads of different types (e.g., assembled nucleotide reads and SBS reads) to generate more accurate genotype calls (thereby reducing false positives and false negatives) corresponding to SNPs and indels. Also contributing to the accuracy improvements over prior systems, the call integration system can utilize different instances of a genotype-call-integration machine-learning model trained for different variant types (e.g., SNPs and indels) to generate or predict genotype calls from multiple read types, something prior systems cannot do. Further contributing to the improved accuracy in genotype calling, in some cases, the call integration system determines and utilizes specific sequencing metrics (unique from prior systems) as a basis for generating calls (e.g., as input data) via the genotype-call-integration machine-learning model.

To accomplish the aforementioned improved accuracies, the call integration system utilizes an improved and unique machine-learning model—the genotype-call-integration machine-learning model—that is trained to perform new applications. Unlike existing variant callers that generate genotype calls from general, single-stream sequencing data—without adjustment or emphasis on whether a particular genomic coordinate historically exhibits or has been detected to exhibit a particular variant—the call integration system utilizes (multiple instances of) a unique genotype-call-integration machine-learning model that generates specific predictions or classifications for different types of variants (e.g., SNPs and indels) from multi-read-type data. In some cases, the call integration system utilizes the genotype-call-integration machine-learning model as a post processing filter to either (i) select between a first genotype call corresponding to a first type of nucleotide reads and a second genotype call corresponding to a second type of nucleotide reads or (ii) determine another genotype call differing from the first genotype call and the second genotype call.

Contributing at least in part to the improved accuracy, the call integration system exhibits improved flexibility over existing sequencing systems. For example, while many existing sequencing systems are limited to analyzing read data from one read type at a time, in some embodiments, the call integration system adapts to processing multiple read types to merge data and generate output genotype calls for particular genomic coordinates or regions. Specifically, unlike some existing sequencing systems, the call integration system can generate genotype calls (e.g., including variant calls) for genomic coordinates based on multiple types of read data for the genomic coordinates, such as assembled nucleotide reads and SBS reads.

In addition to improved accuracy and flexibility, in certain embodiments, the call integration system improves computing efficiency and speed. As noted above, some existing sequencing systems utilize computationally expensive, slow neural network architectures (e.g., deep learning architectures, such as convolutional neural networks) that require many hours (e.g., up to 24 hours) across multiple high-end processors to implement for processing read data to generate calls for a genomic sample. In addition, the call integration system can generate (merged) variant call files by updating only certain fields, without regenerating entirely new variant call files (as done by some prior systems). Such deep learning architectures can further require several days (or weeks) to train. Conversely, the call integration system utilizes a comparatively lightweight, fast architecture for the genotype-call-integration machine-learning model. In contrast to the many hours across multiple processors required by existing sequencing systems, the call integration system requires under an hour (e.g., around fifteen minutes for the call generation model and less than one minute for the genotype-call-integration machine-learning model) of runtime (e.g., on a single processor) to generate genotype calls (and/or variant calls) for a genomic sample. Thus, the call integration system is far faster and less computationally expensive than many deep learning approaches to genotype/variant calling. Indeed, not only are the models of the call integration system faster and less computationally expensive to implement, but the genotype-call-integration machine-learning model is also much faster and less computationally expensive to train than many existing deep learning systems.

As a further advantage over existing sequencing systems, in certain implementations, the call integration system can identify or facilitate changes to individual sequencing metrics that affect the accuracy of genotype calls (and corresponding variant calls). While neural network architectures of many existing sequencing systems render interpretation of internal model data impossible with hidden, latent features among their many layers and neurons, the call integration system utilizes model architectures that facilitate interpretation of the effect of individual sequencing metrics. More specifically, in some cases, the call integration system utilizes a call generation model and a genotype-call-integration machine-learning model that enable much easier extraction and analysis of individual sequencing metrics used throughout the process of generating a genotype call. Indeed, the call integration system can determine respective importance measures for sequencing metrics involved in determining a genotype call at a particular region of genomic coordinates.

As suggested by the foregoing discussion, this disclosure utilizes a variety of terms to describe features and benefits of the call integration system. Additional detail is hereafter provided regarding the meaning of these terms as used in this disclosure. As used in this disclosure, for instance, the term “sample nucleotide sequence” or “sample sequence” refers to a sequence of nucleotides isolated or extracted from a sample organism (or a copy of such an isolated or extracted sequence). In particular, a sample nucleotide sequence includes a segment of a nucleic acid polymer that is isolated or extracted from a sample organism and composed of nitrogenous heterocyclic bases. For example, a sample nucleotide sequence can include a segment of deoxyribonucleic acid (DNA), ribonucleic acid (RNA), or other polymeric forms of nucleic acids or chimeric or hybrid forms of nucleic acids noted below. More specifically, in some cases, the sample nucleotide sequence is found in a sample prepared or isolated by a kit and received by a sequencing device.

Relatedly, as used herein, the term “genomic sample” refers to a target genome or portion of a genome undergoing an assay or sequencing. For example, a genomic sample includes one or more sequences of nucleotides isolated or extracted from a sample organism (or a copy of such an isolated or extracted sequence). In particular, a genomic sample includes a full genome that is isolated or extracted (in whole or in part) from a sample organism and composed of nitrogenous heterocyclic bases. A genomic sample can include a segment of deoxyribonucleic acid (DNA), ribonucleic acid (RNA), or other polymeric forms of nucleic acids or chimeric or hybrid forms of nucleic acids noted below. In some cases, the genomic sample is found in a sample prepared or isolated by a kit and received by a sequencing device.

As further used herein, the term “genotype call” refers to a determination or prediction of a particular genotype of a genomic sample at a genomic locus. In particular, a genotype call can include a prediction of a particular genotype of a genomic sample with respect to a reference genome or a reference sequence at a genomic coordinate or a genomic region. For instance, in some cases, a genotype call includes a determination or prediction that a genomic sample comprises both a nucleobase and a complementary nucleobase at a genomic coordinate that is either homozygous or heterozygous for a reference base or a variant (e.g., homozygous reference bases represented as 0|0 or heterozygous for a variant on a particular strand represented as 0|1). Accordingly, a genotype call can include a prediction of a variant or reference base for one or more alleles of a genomic sample and indicate zygosity with respect to a variant or reference base. A genotype call is often determined for a genomic coordinate or genomic region at which an SNP, insertion, deletion, or other variant has been identified for a population of organisms.

In certain cases, an “initial genotype call” refers to a genotype call corresponding to, or determined from, nucleotide-read data and/or sequencing metrics for a particular type of nucleotide read. For instance, an initial genotype call can include a first genotype call corresponding to a first type of nucleotide reads of a first threshold number of nucleobases and/or a second genotype call corresponding to a second type of nucleotide reads of a second threshold number of nucleobases. By contrast, an “output genotype call” refers to a genotype call reported by or generated for an output data file. For instance, an output genotype call includes a final genotype call that is determined based on one or both of genotype probabilities and variant call classifications from a genotype-call-integration machine-learning model and included in a variant call file (VCF).

As further used herein, the term “nucleobase call” (or simply “base call”) refers to a determination or prediction of a particular nucleobase (or nucleobase pair) for an oligonucleotide (e.g., nucleotide read) during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or another base-call-output file—based on nucleotide reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, a thymine (T) call, or a uracil (U) call.

Relatedly, as used herein, the term “nucleotide read” refers to an inferred sequence of one or more nucleotide bases (or nucleobase pairs) from all or part of a sample nucleotide sequence (e.g., a sample genomic sequence, complementary DNA). In particular, a nucleotide read includes a determined or predicted sequence of nucleobase calls for a nucleotide fragment (or group of monoclonal nucleotide fragments) from a sequencing library corresponding to a genomic sample. For example, in some embodiments, the call integration system determines a nucleotide read by generating nucleobase calls for nucleobases passed through a nanopore of a nucleotide-sample slide, determined via fluorescent tagging, or determined from a well in a flow cell. In some cases, a nucleotide read can refer to a particular type of read, such as a nucleotide read synthesized from sample library fragments that are shorter than a threshold number of nucleobases (e.g., SBS reads). In these or other cases, another type of nucleotide read can refer to (i) assembled nucleotide reads that have been assembled from shorter nucleotide reads to form a contiguous sequence (e.g., assembled nucleotide reads) satisfying a threshold number of nucleobases, (ii) circular consensus sequencing (CCS) reads satisfying the threshold number of nucleobases, or (iii) nanopore long reads satisfying the threshold number of nucleobases.

As noted above, in some embodiments, the call integration system determines sequencing metrics for nucleobase calls of nucleotide reads. As used herein, the term “sequencing metric” refers to a quantitative measurement or score indicating a degree to which an individual nucleobase call (or a sequence of nucleobase calls) aligns, compares, or quantifies with respect to a genomic coordinate or genomic region of a reference genome, with respect to nucleobase calls from nucleotide reads, or with respect to external genomic sequencing or genomic structure. For instance, a sequencing metric includes a quantitative measurement or score indicating a degree to which (i) individual nucleobase calls align, map, or cover a genomic coordinate or reference base of a reference genome; (ii) nucleobase calls compare to reference or alternative nucleotide reads in terms of mapping, mismatch, base call quality, or other raw sequencing metrics; or (iii) genomic coordinates or regions corresponding to nucleobase calls demonstrate mappability, repetitive base call content, DNA structure, or other generalized metrics.

Along these lines, the call integration system determines various types of sequencing metrics from different sources, such as read-based sequencing metrics, externally sourced sequencing metrics, and call-model-generated sequencing metrics. As used herein, the term “read-based sequencing metrics” refers to sequencing metrics derived from nucleotide reads of a sample nucleotide sequence. For example, read-based sequencing metrics include sequencing metrics determined by applying statistical tests to detect differences between a reference sequence and nucleotide reads. In some embodiments, read-based sequencing metrics can include a comparative-mapping-quality-distribution metric that indicates a comparison between mapping qualities or a comparative-mismatch-count metric that indicates a comparison between mismatch counts. In some cases, read-based sequencing metrics can corresponding to nucleobase calls generated from different read types, such as assembled nucleotide reads and/or SBS reads.

By contrast, “externally sourced sequencing metrics” refer to sequencing metrics identified or obtained from one or more external databases. For example, externally sourced sequencing metrics include metrics relating to mappability of nucleotides, replication timing, or DNA structure that are available outside of the call integration system.

Further, the term “call-model-generated sequencing metrics” refers to internal, model-specific sequencing metrics generated or extracted by a call generation model. For example, call-model-generated sequencing metrics include variant calling sequencing metrics extracted or determined via variant caller components of a call generation model and mapping-and-alignment sequencing metrics extracted or determined via mapping-and-alignment components of a call generation model. As indicated above, call-model-generated sequencing metrics can include alignment metrics that quantify a degree to which sample nucleic acid sequences align with genomic coordinates of an example nucleic acid sequence, such as deletion-size metrics or mapping-quality metrics. Further, call-model-generated sequencing metrics can include depth metrics that quantify the depth of nucleobase calls for sample nucleic acid sequences at genomic coordinates of an example nucleic acid sequence, such as forward-reverse-depth metrics or normalized-depth metrics. Call-model-generated sequencing metrics can also include call-quality metrics that quantify a quality or accuracy of nucleobase calls, such as nucleobase-call-quality metrics, callability metrics, or somatic-quality metrics.

As further used herein, the term “genomic coordinate” (or sometimes simply “coordinate”) refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). In some cases, a genomic coordinate refers to a genomic coordinate on a sex chromosome (e.g., chrX or chrY). Consequently, the call integration system can determine genotype probabilities and/or variant call classifications for a genotype call (e.g., a variant call) for a genomic coordinate on a sex chromosome. Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleobase within the source for the reference genome (e.g., mt:16568 or SARS-CoV-2:29001). By contrast, in certain cases, a genomic coordinate refers to a position of a nucleobase within a reference genome without reference to a chromosome or source (e.g., 29727).

Also, as used herein, the term “genomic region” refers to a range of genomic coordinates. Like genomic coordinates, in certain embodiments, a genomic region may be identified by an identifier for a chromosome and a particular position or positions, such as numbered positions following the identifier for a chromosome (e.g., chr1:1234570-1234870).

As noted above, a genomic coordinate includes a position within a reference genome. Such a position may be within a particular reference genome. As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequenced determined by scientists as representative of an organism of a particular species. For example, a linear human reference genome may be GRCh38 or other versions of reference genomes from the Genome Reference Consortium. As a further example, a reference genome may include a reference graph genome that includes both a linear reference genome and paths representing nucleic acid sequences from ancestral haplotypes, such as Illumina DRAGEN Graph Reference Genome hg19.

Additionally, as used herein, the term “reference multigenome” (sometimes referred to as a “graph reference genome”) refers to a reference genome that includes both a linear reference genome and alternate contiguous sequences (or graph augmentations) representing variant haplotype sequences or other variant or alternative nucleic-acid sequences. For instance, a reference multigenome can include a linear reference genome and alternate contiguous sequences corresponding to one or more population haplotype sequences identified from a genomic sample database. As an example, a reference multigenome may include the Illumina DRAGEN Graph Reference Genome hg19.

As further used herein, the term “contiguous sequence” (or “contig assembly”) refers to a consensus nucleotide sequence for a genomic region of a genomic sample (or multiple genomic samples of a species) based on a set of overlapping nucleotide segments corresponding to the genomic region. In particular, a contiguous sequence includes a consensus nucleotide sequence for a genomic region of one or more genomic samples based on nucleotide reads for the one or more genomic samples covering (or overlapping with) the genomic region. As noted above, the terms “contiguous sequence” and “contig assembly” can be used interchangeably.

Relatedly, the term “alternate contiguous sequence” (or simply “alt contig”) refers to a contiguous sequence representing a population haplotype added to a linear reference genome (or other reference genome) at a particular genomic coordinate or genomic coordinates (e.g., lifted over to the linear reference genome). In some implementations, a graph reference genome (or a reference multigenome) can include alternate contiguous sequences mapped to genomic coordinates of a primary assembly for a linear reference genome. For example, an alternate contiguous sequence may represent a population haplotype containing a variant with liftover to two or more genomic coordinates in the linear reference genome corresponding to two or more flanks of variant breakends. In some cases, a hash table for a graph reference genome (or a reference multigenome) includes identifiers that associate alternate contiguous sequences representing variant haplotypes with genomic coordinates representing reference haplotypes from a primary assembly for a linear reference genome.

As used herein, the term “base-call-quality metric” refers to a specific score or other measurement indicating an accuracy of a nucleobase call. In particular, a base-call-quality metric comprises a value indicating a likelihood that one or more predicted nucleobase calls for a genomic coordinate contain errors. For example, in certain implementations, a base-call-quality metric can comprise a Q score (e.g., a PHil's Read EDitor (PHRED) quality score) predicting the error probability of any given nucleobase call. To illustrate, a quality score (or Q score) may indicate that a probability of an incorrect nucleobase call at a genomic coordinate is equal to 1 in 100 for a Q20 score, 1 in 1,000 for a Q30 score, 1 in 10,000 for a Q40 score, etc.

Relatedly, in some embodiments, the call integration in some embodiments, the call integration system can generate sequencing metrics through modifying or updating previous metrics. Such “re-engineered sequencing metrics” can refer to sequencing metrics that have been updated, modified, augmented, refined, or re-engineered to measure or compare nucleobase calls (e.g., nucleobase calls for reads, genotypes, or variant calls) with respect to other nucleobase calls, a standard or reference, or for targeted for a particular objective or task. For example, re-engineered sequencing metrics can include modifications to, or combinations of, raw (e.g., unmodified) sequencing metrics. In some embodiments, for instance, the call integration system generates one or more of the read-based sequencing metrics, the externally sourced sequencing metrics, and/or the call-model-generated sequencing metrics as re-engineered sequencing metrics. In some cases, re-engineered sequencing metrics refer to sequencing metrics that are generated by the call integration system and are therefore proprietary or internal to the call integration system and not available to third-party systems. Example re-engineered sequencing metrics include a comparative-mapping-quality-distribution metric indicating a comparison between mapping quality distributions associated with a reference sequence and alternatives supporting nucleotide reads or a comparative-base-quality metric indicating comparisons between base qualities of a reference sequence and alternative supporting nucleotide reads.

As suggested above, the call integration system can utilize a machine learning model to modify sequencing metrics and update a nucleobase call. As used herein, the term “machine learning model” refers to a computer algorithm or a collection of computer algorithms that automatically improve for a particular task through experience based on use of data. For example, a machine learning model can utilize one or more learning techniques to improve in accuracy and/or effectiveness. Example machine learning models include various types of decision trees (e.g., gradient boosted trees), support vector machines, Bayesian networks, or neural networks.

In some cases, the call integration system utilizes a genotype-call-integration machine-learning model to generate, modify, or update predictions for genotype calls based on sequencing metrics. As used herein, the term “genotype-call-integration machine-learning model” refers to a machine learning model that generates predictions, such as genotype probabilities and/or variant call classifications, for one or more genomic samples. As indicated above, a genotype-call-integration machine-learning model includes a machine learning model that generates predictions for genotype calls of one or more genomic samples based on data from different types of nucleotide reads. For example, in some cases, the genotype-call-integration machine-learning model is trained to generate genotype probabilities indicating probabilities or likelihoods of various genotypes at one or more genomic coordinates based on sequencing metrics. As another example, the genotype-call-integration machine-learning model is trained to generate variant call classifications indicating various probabilities or predictions for variant calls based on sequencing metrics. In some cases, the genotype-call-integration machine-learning model is a series of gradient boosted decision trees (e.g., XGBoost algorithm or treelite algorithm for an ensemble of decision trees), while in other cases the genotype-call-integration machine-learning model is a random forest model, a multilayer perceptron, a linear regression, a support vector machine, a deep tabular learning architecture, a deep learning transformer (e.g., self-attention-based-tabular transformer), or a logistic regression. In certain embodiments, a genotype-call-integration machine-learning model includes multiple sub-models or operates in tandem with another (instance of the) genotype-call-integration machine-learning model. For instance, a first genotype-call-integration machine-learning model (e.g., an ensemble of gradient boosted trees) generates a first set of predictions for a first variant type (e.g., SNPs) at a genomic coordinate, and a second genotype-call-integration machine-learning model generates a second set of predictions for a second variant type (e.g., indels) at the genomic coordinate.

Relatedly, the term “variant call classification” refers to a predicted classification from a genotype-call-integration machine-learning model that indicates a probability, score, or other quantitative measurement associated with some aspect of a genotype call (and how the genotype call impacts a variant call) based on one or more sequencing metrics. A variant call classification can include a specialized prediction depending on the application of a genotype-call-integration machine-learning model, such as for predicting indels. For example, variant call classifications can include, but are not limited to, (i) a true-positive variant probability that a genotype call constitutes a true positive variant for one or more genomic coordinates of a genomic sample; (ii) a zygosity-error probability that a genotype call comprises a genotype-zygosity error at one or more genomic coordinates; or (iii) a reference probability of a homozygous reference genotype at one or more genomic coordinates. Accordingly, the term “reference probability” can refer to a probability of a homozygous reference genotype occurring at one or more genomic coordinates. As explained below, in some cases, a genotype-call-integration machine-learning model generates variant call classifications based on a first type of nucleotide reads (e.g., SBS reads) and a second type of nucleotide reads (e.g., assembled nucleotide reads).

As further used herein, the term “genotype probability” refers to a likelihood, probability, or score of a particular genotype at a genomic coordinate or genomic region. For instance, a genotype probability includes a likelihood of a homozygous reference genotype, a likelihood of a heterozygous variant genotype, or a likelihood of a homozygous variant genotype at one or more genomic coordinates. In some cases, a genotype probability can refer to a posterior genotype probability. Accordingly, in some cases, a genotype probability determined by a genotype-call-integration machine-learning model can be presented in (or modified to be presented in) a posterior genotype probability (GP) field of a VCF, such as a merged VCF. A genotype probability can include a specialized prediction depending on the application of a genotype-call-integration machine-learning model, such as for predicting SNPs.

As noted above, the call integration system can generate genotype probabilities and/or variant call classifications that indicate or reflect a likelihood of identifying a variant at a genomic coordinate. As used herein, the term “variant” refers to a nucleobase or multiple nucleobases that do not align with, differs from, or varies from a corresponding nucleobase (or nucleobases) in a reference sequence or a reference genome. For example, a variant includes a SNP, an indel, or a structural variant that indicates nucleobases in a sample nucleotide sequence that differ from nucleobases in corresponding genomic coordinates of a reference sequence.

As mentioned, in some embodiments, the call integration system modifies data fields corresponding to a variant call file. As used herein, the term “variant call file” refers to a digital file that indicates or represents one or more nucleobase calls and/or variant calls compared to a reference genome along with other information pertaining to the calls. In some cases, a variant call file can also include a genotype call for a genomic sample indicating a reference call or variant call for alleles at particular genomic coordinates or regions. For example, a variant call format (VCF) file refers to a text file format that contains information about variants at specific genomic coordinates, including meta-information lines, a header line, and data lines where each data line contains information about a single nucleobase call (e.g., a single variant). As described further below, the call integration system can generate different versions of variant call files, including a pre-filter variant call file comprising variant nucleobase calls that either pass or fail a quality filter for base-call-quality metrics or a post-filter variant call file comprising variant nucleobase calls that pass the quality filter but excludes variant nucleobase calls that fail the quality filter.

Relatedly, a “merged variant call file” refers to a variant call file generated from one or more other variant call files. For example, a merged variant call file refers to a variant call file generated by selecting or merging data from a variant call file associated with one or more genotype calls determined from a first type of nucleotide reads and a variant call file associated with one or more genotype calls determined from a second type of nucleotide reads. In some cases, a merged variant call file includes some data selected from one initial variant call file and other data selected from a different, initial variant call file. Additionally, a merged variant call file can include data from merged positions, where some fields are generated to include new data not found in other (e.g., non-merged) variant call files. Accordingly, in some embodiments, a merged variant call file is generated from initial variant call files associated with different types of nucleotide calls.

In some embodiments, the call integration system modifies data fields corresponding to metrics of a nucleobase call associated with a variant call file, such as fields for call quality, genotype, and genotype quality. As used herein, the term “call quality” when used with respect to a data field in a variant call file refers to a measure or an indication of a likelihood or a probability that a variant exists at a given location. Accordingly, a call quality field (or QUAL field) corresponding to a VCF file may include a base-call-quality metric, such as a PHRED-scaled quality or Q score, representing a probability that a genomic coordinate of a sample genome includes a variant. Similarly, a “genotype quality” when used with respect to a field refers to a likelihood or a probability that a particular predicted genotype for a nucleobase call is correct.

As noted, in some embodiments, the call integration system utilizes a call generation model to generate a nucleobase call for a genomic coordinate. As used herein, the term “call generation model” refers to a probabilistic model that generates sequencing data from nucleotide reads of a sample nucleotide sequence, including nucleobase calls, variant calls, and/or genotype calls along with associated metrics. Accordingly, in some cases, a call generation model may be a variant call generation model. For example, in some cases, a call generation model refers to a Bayesian probability model that generates variant calls based on nucleotide reads of a sample nucleotide sequence. Such a model can process or analyze sequencing metrics corresponding to read pileups (e.g., multiple nucleotide reads corresponding to a single genomic coordinate), including mapping quality, base quality, and various hypotheses including foreign reads, missing reads, joint detection, and more. A call generation model may likewise include multiple components, including, but not limited to, different software applications or components for mapping and aligning, sorting, duplicate marking, computing read pileup depths, and variant calling. In some cases, a call generation model refers to an ILLUMINA DRAGEN model for variant calling functions and mapping and alignment functions (e.g., a DRAGEN variant caller or “DRAGEN VC”).

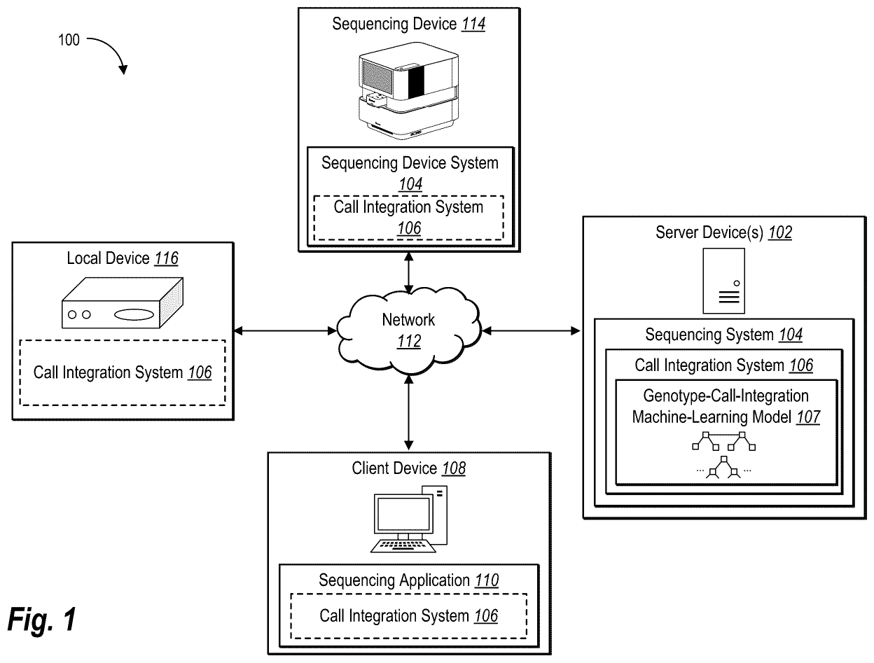

The following paragraphs describe the call integration system with respect to illustrative figures that portray example embodiments and implementations. For example, FIG. 1 illustrates a schematic diagram of a system environment (or “environment”) 100 in which a call integration system 106 operates in accordance with one or more embodiments. As illustrated, the environment 100 includes one or more server device(s) 102 connected to a client device 108, a local device 116, and a sequencing device 114 via a network 112. While FIG. 1 shows an embodiment of the call integration system 106, this disclosure describes alternative embodiments and configurations below.

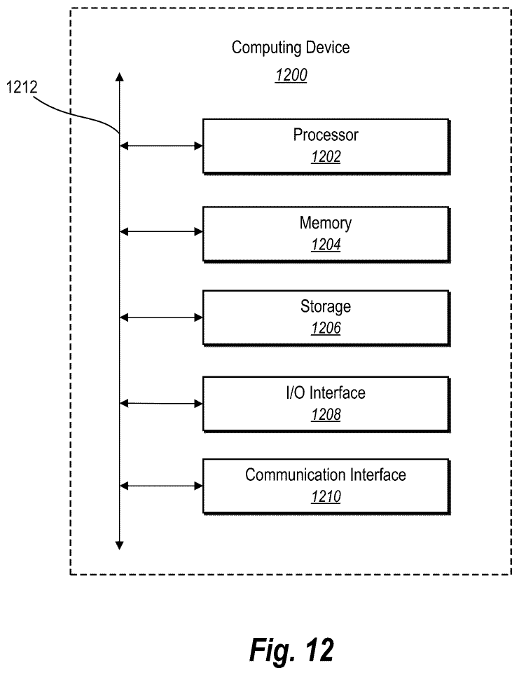

As shown in FIG. 1, the server device(s) 102, the client device 108, the local device 116, and the sequencing device 114 can communicate with each other via the network 112. The network 112 comprises any suitable network over which computing devices can communicate. Example networks are discussed in additional detail below with respect to FIG. 12.

As indicated by FIG. 1, the sequencing device 114 comprises a device for sequencing a nucleic acid polymer. In some embodiments, the sequencing device 114 analyzes nucleic acid segments or oligonucleotides extracted from genomic samples to generate nucleotide reads or other data utilizing computer implemented methods and systems (described herein) either directly or indirectly on the sequencing device 114. More particularly, the sequencing device 114 receives and analyzes, within nucleotide-sample slides (e.g., flow cells), nucleic acid sequences extracted from genomic samples. In one or more embodiments, the sequencing device 114 utilizes SBS to sequence nucleic acid polymers into nucleotide reads. In addition or in the alternative to communicating across the network 112, in some embodiments, the sequencing device 114 bypasses the network 112 and communicates directly with the client device 108.

As further indicated by FIG. 1, the local device 116 is located at or near a same physical location of the sequencing device 114. Indeed, in some embodiments, the local device 116 and the sequencing device 114 are integrated into a same computing device. The local device 116 may run the call integration system 106 to generate, receive, analyze, store, and transmit digital data, such as by receiving sequencing metrics or determining genotype calls and/or variant calls based on analyzing such sequencing metrics. As shown in FIG. 1, the sequencing device 114 may send (and the local device 116 may receive) sequencing metrics generated during a sequencing run of the sequencing device 114. By executing software in the form of the call integration system 106, the local device 116 may align nucleotide reads with a reference genome and/or utilize a genotype-call-integration machine-learning model 107 to determine genotypes and/or genetic variants based on the sequencing metrics. The local device 116 may also communicate with the client device 108. In particular, the local device 116 can send data to the client device 108, including a variant call file (VCF), sequencing metrics, or other information indicating nucleobase calls, genotype calls, variant calls, sequencing metrics, error data, or other metrics.

As further indicated by FIG. 1, the server device(s) 102 may generate, receive, analyze, store, and transmit digital data, such as data for determining genotype calls or sequencing nucleic acid polymers. As shown in FIG. 1, the sequencing device 114 may send (and the server device(s) 102 and/or the local device 116 may receive) call data and/or sequencing metrics. The server device(s) 102 may also communicate with the client device 108 and/or the local device 116. In particular, the server device(s) 102 and/or the local device 116 can send data to the client device 108, including a variant call file or other information indicating nucleobase calls, genotype calls, variant calls, sequencing metrics, error data, or other metrics.

In some embodiments, the server device(s) 102 comprise a distributed collection of servers where the server device(s) 102 include a number of server devices distributed across the network 112 and located in the same or different physical locations. Further, the server device(s) 102 can comprise a content server, an application server, a communication server, a web-hosting server, or another type of server. In some cases, the server device(s) 102 are located at a same physical location as the sequencing device 114 and/or the local device 116.

As further shown in FIG. 1, the server device(s) 102 and/or the sequencing device 114 can include a sequencing system 104. Generally, the sequencing system 104 analyzes read data and/or call data, such as sequencing metrics received from the sequencing device 114, to determine nucleobase sequences for nucleic acid polymers. For example, the sequencing system 104 can receive raw data from the sequencing device 114 and can determine a nucleobase sequence for a nucleic acid segment. In some embodiments, the sequencing system 104 determines the sequences of nucleobases in DNA and/or RNA segments or oligonucleotides. In addition to processing and determining sequences for nucleic acid polymers, the sequencing system 104 also generates a variant call file indicating one or more genotype calls and/or variant calls for one or more genomic coordinates.

As just mentioned, and as illustrated in FIG. 1, the call integration system 106 analyzes call data, such as sequencing metrics from the sequencing device 114, to determine genotype calls for sample nucleotide sequences of a genomic sample. The call integration system 106 includes a call generation model and a genotype-call-integration machine-learning model 107. In some embodiments, the call integration system 106 determines sequencing metrics for sample nucleotide sequences. Based on data derived or prepared from the sequencing metrics, the call integration system 106 trains and/or applies a call generation model to determine nucleobase calls for the sample sequence corresponding to genomic coordinates. The call integration system 106 further utilizes a genotype-call-integration machine-learning model 107 to generate sets of predictions (e.g., genotype probabilities for SNPs or variant call classifications for indels) to update or modify the genotype calls (and/or variant calls). Based on such data, for example, the call integration system 106 can update data fields corresponding to a variant call file to update a genotype call and/or a variant call for improved accuracy.

As further illustrated and indicated in FIG. 1, the client device 108 can generate, store, receive, and send digital data. In particular, the client device 108 can receive sequencing metrics from the sequencing device 114. Furthermore, the client device 108 may communicate with the server device(s) 102 and/or the local device 116 to receive a variant call file comprising genotype calls and/or other metrics, such as a call-quality and/or a genotype quality. The client device 108 can accordingly present or display information pertaining to the genotype call within a graphical user interface to a user associated with the client device 108. For example, the client device 108 can present a contribution-measure interface that includes a visualization or a depiction of various contribution measures associated with, or attributed to, individual sequencing metrics with respect to a particular nucleobase call.

The client device 108 illustrated in FIG. 1 may comprise various types of client devices. For example, in some embodiments, the client device 108 includes non-mobile devices, such as desktop computers or servers, or other types of client devices. In yet other embodiments, the client device 108 includes mobile devices, such as laptops, tablets, mobile telephones, or smartphones. Additional details regarding the client device 108 are discussed below with respect to FIG. 12.

As further illustrated in FIG. 1, the client device 108 includes a sequencing application 110. The sequencing application 110 may be a web application or a native application stored and executed on the client device 108 (e.g., a mobile application, desktop application). The sequencing application 110 can include instructions that (when executed) cause the client device 108 to receive data from the call integration system 106 and present, for display at the client device 108, data from a variant call file. Furthermore, the sequencing application 110 can instruct the client device 108 to display a visualization of contribution measures for sequencing metrics of a genotype call.

As further illustrated in FIG. 1, the call integration system 106 may be located on the client device 108 as part of the sequencing application 110 or on the sequencing device 114 or on the local device 116. Accordingly, in some embodiments, the call integration system 106 is implemented by (e.g., located entirely or in part) on the client device 108. In yet other embodiments, the call integration system 106 is implemented by one or more other components of the environment 100, such as the sequencing device 114 or the local device 116. In particular, the call integration system 106 can be implemented in a variety of different ways across the server device(s) 102, the network 112, the client device 108, and the sequencing device 114. For example, the call integration system 106 can be downloaded from the server device(s) 102 to the client device 108, to the local device 116, and/or to the sequencing device 114 where all or part of the functionality of the call integration system 106 is performed at each respective device within the environment 100.

Though FIG. 1 illustrates the components of environment 100 communicating via the network 112, in certain implementations, the components of environment 100 can also communicate directly with each other, bypassing the network 112. For instance, and as previously mentioned, in some implementations, the client device 108 communicates directly with the sequencing device 114 and/or the local device 116. Additionally, in some embodiments, the client device 108 communicates directly with the call integration system 106 (hosted on one or more of the illustrated components). Moreover, the call integration system 106 can access one or more databases housed on or accessed by the server device(s) 102 or elsewhere in the environment 100.

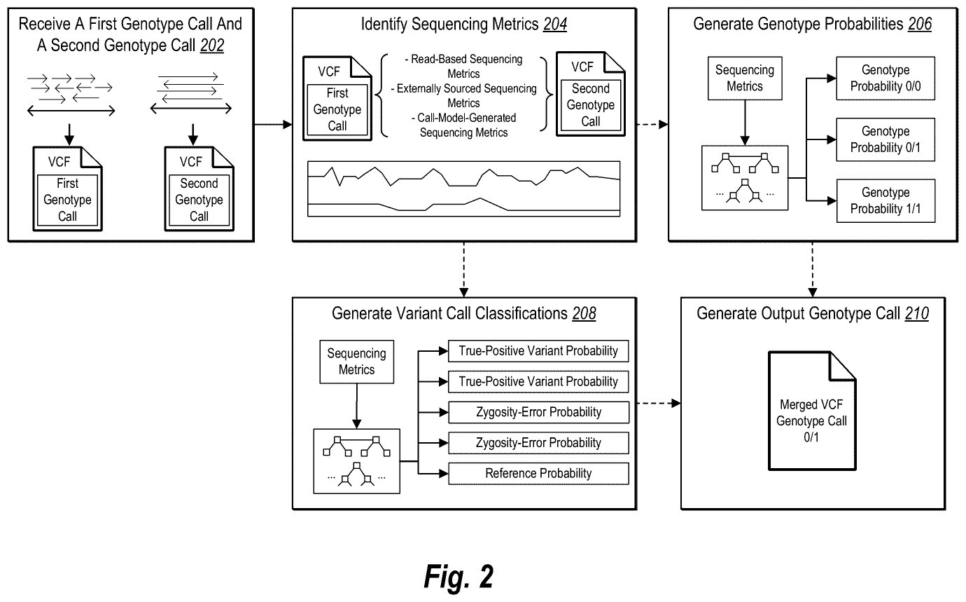

As indicated above, the call integration system 106 can determine an output genotype call based on one or more sequencing metrics for initial genotype calls from different types of nucleotide reads. In particular, the call integration system 106 can generate predictions (e.g., genotype probabilities or variant call classifications) from sequencing metrics utilizing a genotype-call-integration machine-learning model and can determine or update various metrics (e.g., within a VCF file) associated with a genotype call from the generated predictions. In accordance with one or more embodiments, FIG. 2 illustrates an example overview of the call integration system 106 determining an output genotype call based on genotype probabilities or variant call classifications from a genotype-call-integration machine-learning model. Additional detail regarding the acts of FIG. 2 is provided thereafter with reference to subsequent figures.

As illustrated in FIG. 2, the call integration system 106 performs an act 202 to receive a first genotype call and a second genotype call. In particular, in some embodiments, the call integration system 106 receives a first genotype call indicated by a first VCF file generated from nucleotide reads of a first read type. In addition, the call integration system receives a second genotype call indicated by a second VCF file generated from nucleotide reads of a second read type. In some cases, the call integration system 106 generates the first genotype call by analyzing SBS reads—e.g., nucleotide reads synthesized from sample library fragments that are shorter than the first threshold number of nucleobases. In these or other cases, the call integration system 106 generates the second genotype call by analyzing a different type of read data, such as: (i) assembled nucleotide reads—nucleotide reads that have been assembled from shorter nucleotide reads to form a contiguous sequence, (ii) CCS reads, and/or (iii) nanopore long reads. In certain embodiments, the first and second received genotype calls are initial genotype calls that the call integration system 106 uses as a basis for ultimately generating an output genotype call (e.g., by merging data associated with the first and second genotype calls).

As also illustrated in FIG. 2, the call integration system 106 performs an act 204 to identify sequencing metrics. In particular, the call integration system 106 identifies or determines sequencing metrics, such as read-based sequencing metrics, externally sourced sequencing metrics, and call-model-generated sequencing metrics. For example, the call integration system 106 determines sequencing metrics that indicate various attributes or data in relation to various genotype calls of nucleotide reads from a sample nucleotide sequence. In some embodiments, the call integration system 106 determines or extracts different sequencing metrics for generating genotype calls associated with different variant types, such as SNPs and indels. Indeed, from the different sequencing metrics, the call integration system 106 can generate an output genotype call corresponding to the respective variant type on which a genotype-call-integration machine-learning model is trained.

To elaborate, as illustrated in FIG. 2, the call integration system 106 utilizes different instances of a genotype-call-integration machine-learning model to generate different predictions for different variant types based on extracted sequencing metrics. For example, to generate an output genotype call corresponding to a (biallelic) SNP, the call integration system 106 performs an act 206 to generate genotype probabilities. As another example, to generate an output genotype call corresponding to an indel (or a multiallelic SNP or a variant type other than a biallelic SNP), the call integration system 106 performs an act 208 to generate variant call classifications. As indicated below, in some embodiments, the call integration system 106 can use one or both of a SNP-specific genotype-call-integration machine-learning model to generate genotype probabilities and an indel-specific genotype-call-integration machine-learning model to generate variant call classifications. In some cases, the call integration system 106 can use a biallelic-SNP-genotype-call-integration machine-learning model to analyze or determine biallelic SNPs. In these or other cases, the call integration system 106 can use an indel-specific genotype-call-integration machine-learning model to analyze or determine indels, multiallelic SNPs, or other variant types that are not biallelic SNPs.

To generate the genotype probabilities (e.g., via the act 206), the call integration system 106 utilizes a genotype-call-integration machine-learning model to analyze sequencing metrics (e.g., SNP-related sequencing metrics). Specifically, the call integration system 106 generates the genotype probabilities for one or more candidate SNPs utilizing the genotype-call-integration machine-learning model trained with SNP training data. From the sequencing metrics, the genotype-call-integration machine-learning model generates a set of genotype probabilities for a particular genomic coordinate, indicating a likelihood of a 0/0 genotype (e.g., a homozygous reference genotype), a likelihood of a 0/1 genotype or a I/O genotype (e.g., a heterozygous genotype), and a likelihood of a 1/1 genotype (e.g., a homozygous alternate genotype).

To generate the variant call classifications (e.g., via the act 208), the call integration system 106 generates (or updates or refines) variant call classifications from sequencing metrics utilizing a genotype-call-integration machine-learning model. To elaborate, the call integration system 106 utilizes the genotype-call-integration machine-learning model to process or analyze one or more sequencing metrics and to generate a set of classifications (e.g., predicted probabilities associated with variants, zygosity, or reference calls). For instance, the call integration system 106 generates, utilizing the genotype-call-integration machine-learning model, a set of variant call classifications, including: i) a first true-positive variant probability for the first genotype call (e.g., from the first read type), ii) a second true-positive variant probability for the second genotype call (e.g., from the second read type), iii) a first zygosity-error probability for the first genotype call, iv) a second zygosity-error probability for the second genotype call, and v) a reference probability.

As further illustrated in FIG. 2, the call integration system 106 also performs an act 210 to generate an output genotype call. In particular, the call integration system 106 generates an output genotype call for one or more genomic coordinates of an SNP based on genotype probabilities output by a genotype-call-integration machine-learning model. Additionally, or alternatively, the call integration system 106 generates an output genotype call for one or more genomic coordinates of an indel based on variant call classifications output by a genotype-call-integration machine-learning model. For either SNPs or indels, the call integration system 106 determines or updates a genotype call by generating a merged VCF file that merges, or is otherwise generated from, data associated with a first read type (e.g., SBS reads) and data associated with a second read type (e.g., assembled nucleotide reads). In some cases, the call integration system 106 determines an output genotype indicating a presence or absence of an SNP or an indel at the one or more genomic coordinates of a genomic sample. For instance, the call integration system 106 selects an initial genotype call (e.g., the first or second genotype call) as the output genotype call. Alternatively, the call integration system 106 generates an output genotype call different from the initial genotype calls (e.g., the first and second genotype calls), but based on data associated with the initial genotype calls.

In some embodiments, the call integration system 106 utilizes a call generation model to generate a merged VCF file from genotype probabilities and/or variant call classifications (as generated by a genotype-call-integration machine-learning model). For example, the call integration system 106 applies a number of Bayesian probabilistic models or algorithms to derive various probabilities for different nucleobases, quality metrics, mapping metrics, joint metrics, and other data occurring within the sample nucleotide sequence to include within a variant call file. From the probabilistic models, the call integration system 106 can further determine an output genotype call that indicates a predicted genotype (or variant) for the sample genome at a genomic coordinate corresponding to a reference genome.

As part of generating an output genotype call, in certain implementations, the call integration system 106 utilizes the genotype probabilities and/or the variant call classifications to generate, recalibrate, determine, modify, confirm, or augment the initial genotype call(s). To elaborate, the call integration system 106 utilizes the genotype probabilities and/or the variant call classifications (and/or other features) to determine or update certain metrics associated with a genotype call. For example, the call integration system 106 modifies data fields corresponding to a variant call file for metrics, such as call quality, genotype, and genotype quality (or others as described below) to generate an output genotype call (e.g., as a new genotype call or as a modified or merged version of the first genotype call and/or the second genotype call).

Although FIG. 2 illustrates a particular order for the acts 202-210, in some embodiments, the call integration system 106 performs the acts in a different order and/or in tandem simultaneously. For example, the call integration system 106 can perform the act 206 to generate genotype probabilities and/or the act 208 to generate variant call classifications while, or during the process of, performing the act 210 to generate an output genotype call. For example, the call integration system 106 simultaneously implements a genotype-call-integration machine-learning model and a call generation model to generate an output genotype call and genotype probabilities/variant call classifications for modifying the output genotype call. In some cases, the call integration system 106 further modifies data fields corresponding for a merged variant call file of the output genotype call (e.g., within a pre-filter or post-filter variant call file). As suggested above, this simultaneous or parallel operation affords the call integration system 106 improved computational efficiency and increased speed by recalibrating genotype calls as they are initially generated (rather than performing one operation before the other).

In one or more implementations, the call integration system 106 determines the output genotype call as part of genomic coordinate(s) tagged for a SNP or an indel. For example, the call integration system 106 determines an output genotype call to represent an SNP at a genomic coordinate (e.g., chr1:151863125) by identifying a G in the sample nucleotide sequence where an A exists in the reference genome. As another example, the call integration system 106 determines genotype calls surrounding one or more genomic coordinates (e.g., chr1:49263256) indicate a deletion by identifying a single G in the sample nucleotide sequence where GTAAC exists in the reference genome. As a further example, the call integration system 106 determines a sequence of genotype calls represent an insertion at a genomic coordinate (e.g., chr1:7602080) by identifying a sequence of TTTCC in the sample nucleotide sequence where a T exists in the reference genome. Indeed, in some cases, an insertion includes a sequence of genotype calls that replace a single reference base at a genomic coordinate of a reference sequence.

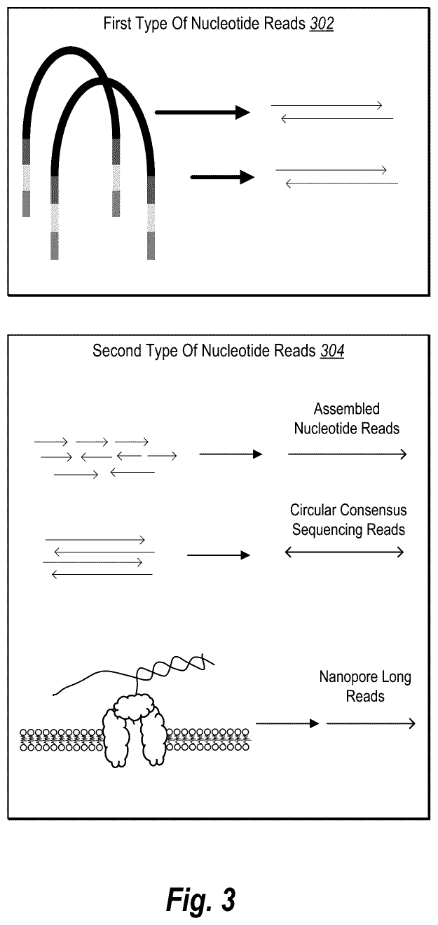

As mentioned above, in certain embodiments, the call integration system 106 receives, identifies, or determines initial genotype calls from different types of nucleotide reads. In particular, the call integration system 106 utilizes a multi-read-type pipeline to merge sequencing metrics or other data from one type of nucleotide read (e.g., short reads or SBS reads) with sequencing metrics or other data from another type of nucleotide read (e.g., long reads or assembled nucleotide reads) to generate an output genotype call from the initial genotype calls. FIG. 3 illustrates example types of nucleotide reads that the call integration system 106 can analyze or receive data concerning as part of generating an output genotype call at a genomic coordinate in accordance with one or more embodiments. As indicated above, in some embodiments, the call integration system 106 identifies or determines genotype calls and corresponding sequencing metrics based on a first type of nucleotide read and a second type of nucleotide read depicted in FIG. 3.

As illustrated in FIG. 3, the call integration system 106 analyzes read data associated with a first type of nucleotide reads 302. In particular, the call integration system 106 receives or determines a first genotype call from the first type of nucleotide reads 302. For example, the call integration system 106 determines or receives a genotype call indicating a genotype or a variant at a particular genomic coordinate, as indicated by reads of the first type of nucleotide reads 302. In some embodiments, the first type of nucleotide reads 302 includes short reads (e.g., reads shorter than a threshold length or made up of fewer than a threshold number of nucleobases), such as SBS reads synthesized from sample library fragments that are shorter than the threshold number of nucleobases. In certain embodiments, the call integration system 106 determines the first type nucleotide reads 302 from wells in a flow cell and/or via fluorescent tagging. In some cases, the call integration system 106 utilizes cluster generation and SBS chemistry to sequence millions or billions of clusters in a flow cell. During SBS chemistry, for each cluster, the call integration system 106 stores nucleobase calls from the nucleotide reads for every cycle of sequencing via real-time analysis (RTA) software. While the particular genomic coordinate described above includes a first genotype call based on reads of the first type of nucleotide reads 302 and a second genotype call based on reads of reads of the second type of nucleotide reads 304, certain genomic coordinates include only genotype calls based on reads of the first type of nucleotide reads 302 or only genotype calls based on reads of the second type of nucleotide reads 304, but not both.

As further illustrated in FIG. 3, the call integration system 106 analyzes read data associated with a second type of nucleotide reads 304. In particular, the call integration system 106 receives or determines a second genotype call from the second type of nucleotide reads 304. For example, the call integration system 106 determines or receives a genotype call indicating a genotype or a variant at a particular genomic coordinate, as indicated by reads of the second type of nucleotide reads 304. More specifically, the second type of nucleotide reads 304 can include long reads (e.g., reads longer than a threshold length or made up of at least a threshold number of nucleobases), such as assembled nucleotide reads, CCS reads, and/or nanopore long reads.

Regarding assembled nucleotide reads, the call integration system 106 can determine assembled nucleotide reads by utilizing a mutagenesis process and a rendering process. To elaborate, the call integration system 106 can assemble, create, synthesize, or generate long reads from short reads. For example, the call integration system 106 can apply mutations to a set of short reads (e.g., SBS reads or other short reads) to introduce unique genetic signatures so that the assembly can work over low complexity regions with many repeats. In some cases, the call integration system 106 applies random mutations and uses the output of mutated short reads to recover information in areas of a sample genome that are difficult to sequence using ordinary SBS techniques. For instance, the call integration system 106 combines mutated short reads to form assembled long reads, and the call integration system 106 further performs a rendering process to undo at least a portion of the mutations after the short reads are combined or assembled into long reads.

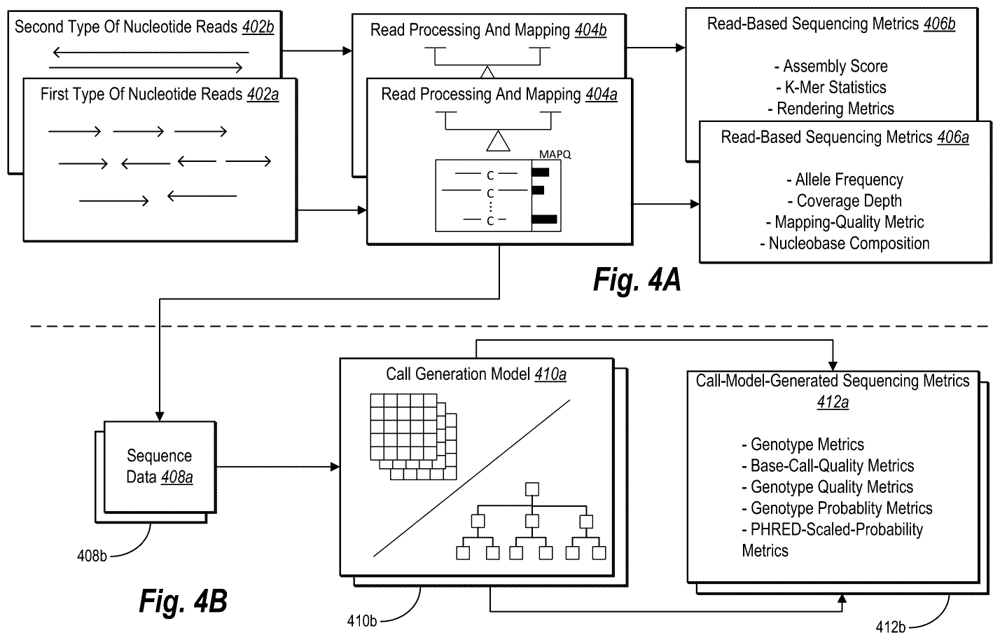

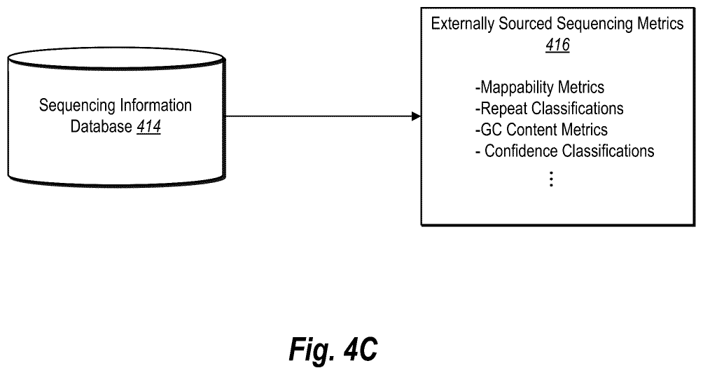

As mentioned above, in certain described embodiments, the call integration system 106 determines or extracts sequencing metrics for genotype calls at genomic coordinates. In particular, the call integration system 106 determines sequencing metrics, such as read-based sequencing metrics, externally sourced sequencing metrics, and call-model-generated sequencing metrics from calls corresponding to nucleotide reads from a sample nucleotide sequence. FIGS. 4A-4C illustrate the call integration system 106 determining sequencing metrics in accordance with one or more embodiments. Specifically, FIG. 4A illustrates the call integration system 106 determining read-based sequencing metrics based on a first type of nucleotide reads and a second type of nucleotide reads; FIG. 4B illustrates the call integration system 106 determining call-model-generated sequencing metrics for genotype calls corresponding to either the first type of nucleotide reads or the second type of nucleotide reads; and FIG. 4C illustrates the call integration system 106 identifying or determining externally sourced sequencing metrics for genomic coordinate(s) for genotype calls corresponding to either the first type of nucleotide reads or the second type of nucleotide reads.

As illustrated in FIG. 4A, the call integration system 106 accesses, retrieves, obtains, determines, receives, or generates nucleotide reads, including a first type of nucleotide reads 402a (e.g., the first type of nucleotide reads 302) and a second type of nucleotide reads 402b (e.g., the second type of nucleotide reads 304). For example, the call integration system 106 determines the nucleotide reads utilizing the sequencing device 114 for regions from a sample nucleotide sequence (e.g., a sample genome). For example, the call integration system 106 generates a plurality of nucleotide reads utilizing sequencing-by-synthesis (SBS) techniques, Sanger sequencing techniques, assembled nucleotide read techniques, or other sequencing techniques discussed herein to determine genotype calls for oligonucleotide clusters.

As further illustrated in FIG. 4A, in some embodiments, the call integration system 106 performs read processing and mapping 404a for the first type of nucleotide reads 402a and performs read processing and mapping 404b for the second type of nucleotide reads 402b. For example, the call integration system 106 utilizes RTA software to store base call data in the form of individual base call data files (or BCLs). In some cases, the call integration system 106 further converts the BCL files into sequence data 408a and 408b (e.g., via BCL to FASTQ conversion), as illustrated in FIG. 4B—where the sequence data 408a corresponds to the first type of nucleotide reads 402a, and the sequence data 408b corresponds to the second type of nucleotide reads 402b.

As shown in FIG. 4A, the call integration system 106 generates multiple-read coverages (e.g., read pileups) that include multiple nucleotide reads or nucleobase calls corresponding to a single genomic coordinate. In particular, in certain embodiments, the call integration system 106 aligns nucleotide reads with a reference genome or receives information pertaining to the read alignment. Specifically, the call integration system 106 determines which nucleobase(s) of a given read align with which genomic coordinate of a reference sequence (or receives information indicating alignment). Different reads have different lengths and include different nucleobases. Accordingly, in some cases, the call integration system 106 analyzes each nucleotide of each read to determine (or receives information indicating) where the read “fits” in relation to a reference sequence—e.g., where the bases within the read align with bases in the reference. In some cases, the call integration system 106 aligns many reads at a single genomic coordinate, thus resulting a read pileup.

In certain embodiments, the call integration system 106 performs additional statistical tests to determine or detect differences between metrics associated with a reference nucleotide sequence and metrics associated with alternative supporting nucleotide reads. Through these statistical tests, the call integration system 106 re-engineers raw sequencing metrics to determine read-based sequencing metrics 406a for the first type of nucleotide reads 402a and read-based sequencing metrics 406b for the second type of nucleotide reads 402b. In some embodiments, the call integration system 106 determines a shared set of sequencing metrics associated with both the first type of nucleotide reads 402a and the second type of nucleotide reads 402b.

In some cases, the call integration system 106 determines or extracts raw sequencing metrics that include one or more of (i) alignment metrics for quantifying alignment of sample nucleotide sequences with genomic coordinates of an example nucleotide sequence (e.g., a reference genome or a nucleotide sequence from an ancestral haplotype), (ii) depth metrics for quantifying depth of nucleobase calls for sample nucleotide sequences at genomic coordinates of the example nucleotide sequence, or (iii) call-quality metrics for quantifying quality of nucleobase calls for sample nucleotide sequences at genomic coordinates of the example nucleotide sequence. For instance, the call integration system 106 determines mapping-quality metrics (e.g., MAPQ metrics), soft-clipping metrics, or other alignment metrics that measure an alignment of sample sequences with a reference genome. As another example, the call integration system 106 determines forward-reverse-depth metrics (or other such depth metrics) or callability metrics for genotype calls or variant calls (or other such call-quality metrics).

As just mentioned, in some embodiments, the call integration system 106 re-engineers the raw sequencing metrics to generate read-based sequencing metrics 406a and 406b that are more informative for comparing metrics associated with a reference nucleotide sequence with metrics associated with various supporting alternative nucleotide reads. For example, the call integration system 106 determines various metrics for a sample sequence in relation to a reference sequence and further determines various metrics for the sample sequence in relation to alternative supporting sequences. In addition, the call integration system 106 performs comparative analyses between metrics associated with the reference sequence and the metrics associated with the alternative supporting reads.

For instance, the call integration system 106 compares mapping of nucleobases of a sample nucleotide sequence (e.g., sample genome) to a reference sequence with mapping of the nucleobases to various alternative supporting reads. In some cases, the call integration system 106 determines mapping qualities associated with the reference sequence to compare with mapping qualities associated with alternative supporting reads. For example, the call integration system 106 determines mapping quality statistics reflecting differences in the distribution of reads supporting a reference sequence versus reads supporting alternative alleles.

In these or other cases, the call integration system 106 determines mismatch counts between the sample sequence and the reference sequence and between the reference sequence and alternative supporting reads. The call integration system 106 further compares the mismatch counts to determine a comparative-mismatch-count metric. Further, the call integration system 106 determines soft-clipping metrics for the sample sequence in relation to the reference sequence and further determines soft-clipping metrics in relation to alternative supporting reads. The call integration system 106 also compares the soft clipping metrics between the reference sequence and the alternative supporting reads to generate a comparative-soft-clipping metric. Further still, the call integration system 106 compares base-call-quality metrics in relation to the reference sequence and alternative supporting reads and/or compares query positions of the sample sequence in relation to the reference sequence with those in relation to alternative supporting reads.

As further illustrated in FIG. 4A, the call integration system 106 utilizes the comparisons and/or other statistical tests to generate the read-based sequencing metrics 406a and 406b. In some cases, the call integration system 106 generates the read-based sequencing metrics 406a and 406b to include one or more of the same metrics enumerated above. For example, from the first type of nucleotide reads 402a and the second type of nucleotide reads 402b, the call integration system 106 generates read-based sequencing metrics 406a and 406b, including: i) an allele frequency metric indicating a frequency of occurrence for an allele of a first genotype call, an allele of a second genotype call, or a different allele of an alternative genotype call differing from the first and second genotype calls, ii) a coverage depth metric indicating a particular (e.g., maximum or cumulative total) depth of coverage for the first type of nucleotide reads 402a corresponding to a first genotype call or the second type of nucleotide reads 402b corresponding to the second genotype call, iii) a mapping-quality metric (e.g., a MAPQ score) for the first type of nucleotide reads 402a corresponding to a first genotype call or the second type of nucleotide reads 402b corresponding to a second genotype call, iv) a nucleobase composition metric indicating a makeup or composition of nucleobases at genomic coordinates of one or more nucleotide reads from the first type of nucleotide reads 402a or the second type of nucleotide reads 402b, and v) an average coverage depth metric indicating an average (e.g., a mean or median) of coverage depth for the first type of nucleotide reads 402a corresponding to a first genotype call or the second type of nucleotide reads 402b corresponding to a second genotype call.

Additionally, the call integration system 106 utilizes the comparisons and statistical tests to generate the read-based sequencing metrics 406b from the second type of nucleotide reads 402b that may not apply to the first type of nucleotide reads 402a. For example, the call integration system 106 generates the read-based sequencing metrics 406b, including: i) an assembly score indicating a measure of accuracy or completeness for assembled reads generated using mutagenesis and rendering, ii) k-mer statistics indicating lengths of reads and/or lengths of variants (e.g., insertions or deletions), and iii) rendering metrics indicating a measure of accuracy or completeness of rendering mutations out of assembled nucleotide reads (e.g., from a mutagenesis process). Additional detail regarding the read-based sequencing metrics 406a and 406b is provided hereafter.

A. Read-Based Sequencing Metrics

The following paragraphs describe various read-based sequencing metrics in more detail. For example, the call integration system 106 determines a base-call quality score for base calls within a nucleotide read. Specifically, the call integration system 106 determines probabilities of correctness for nucleobase calls of nucleotide reads (e.g., PHRED+33 encoded). In some cases, the call integration system 106 determines one or more base-call quality scores in the form of a DRAGEN QUAL score or a Q score for one or more nucleobase calls. Further, the call integration system 106 determines a fraction of nucleotide reads supporting an alternate contiguous sequence from a reference genome. For instance, the call integration system 106 determines numbers of nucleotide reads supporting (e.g., matching or aligning with) an alternate contiguous sequence of a reference genome and numbers of nucleotide reads supporting a primary assembly within the reference genome. The call integration system 106 further compares the aforementioned numbers and determines a fraction to reflect the comparison.

In some cases, the call integration system 106 utilizes specific features to determine the fraction of reads supporting an alternate contiguous sequence, including: i) an alignment score in relation to a reference genome, ii) an alignment score in relation to an assembly of alternate contiguous sequences, iii) a mapping quality of nucleotide reads, and iv) an amount of overlap with a genomic region. In addition, the call integration system 106 can categorize reads based on their alignment according to the following categories: i) perfect alignment to an assembly of alternate contiguous sequences (e.g., satisfying a first alignment score threshold), ii) perfect alignment to a reference genome, iii) strong alignment to an assembly of alternate contiguous sequences (e.g., satisfies a second alignment score threshold but not the first alignment score threshold), iv) strong alignment to a reference genome (e.g., also satisfying the second alignment score threshold but not the first alignment score threshold), and v) no strong alignment either an assembly of alternate contiguous sequences or a reference genome (e.g., fails to satisfy the second alignment threshold in relation to both the assembly of alternate contiguous sequences and the reference genome). Based on these five categories, the call integration system 106 can further determine fractions comparing each of these categories to determine a fraction of nucleotide reads (e.g., a fraction of reads overlapping with a target genomic region) supporting an alternate contiguous sequence versus a fraction of the nucleotide reads supporting a reference genome.

In addition, the call integration system 106 can determine, as a read-based sequencing metric, a number of split nucleotide reads from the nucleotide reads corresponding to the an genotype call or variant call. More particularly, the call integration system 106 determines a number of nucleotide reads with no contiguous alignment (or less than a threshold number of bases that align) with a primary assembly of a reference genome, but that rather contain nucleotide-read fragments that align with two or more reference sequences within the reference genome. For example, the call integration system 106 determines, using a call generation model, a split read count supporting a genotype call. For heterozygous deletion calls, some false positive cases have large split read counts that exceed those in true positive cases, along with a coverage depth that is higher than expected. The call integration system 106 can thus generate a split nucleotide read metric based on the nucleotide reads supporting a genotype call.

In some embodiments, the call integration system 106 compares split read evidence supporting alternate alleles for forward and reverse oriented nucleotide reads, respectively. If most of the evidence is from either the forward or reverse oriented reads, this bias could be indicative of a systematic issue especially when the read count is relatively high (e.g., greater than 10 nucleotide reads). The call integration system 106 uses forward and reverse read counts with perfect alignment scores with the contiguous sequence as sequencing metrics for the genotype-call-integration machine-learning model.

As mentioned above, the call integration system 106 can determine, as a read-based sequencing metric, a coverage depth of the nucleotide reads corresponding to the initial structural variant call. For example, the call integration system 106 determines a count or a number of nucleotide reads that overlap with a target genomic region corresponding to a variant identified as present or absent by an initial genotype call or an initial variant call. Accordingly, coverage depth may be represented by a raw count of nucleotide reads overlapping with a target genomic region by at least a threshold number of nucleotide bases.

Further, the call integration system 106 can determine, as part of the read-based sequencing metrics, an additional genotype call (e.g., variant call) located within a threshold number of base pairs from an initial genotype call (e.g., variant call) within the genomic sample. For example, the call integration system 106 determines a variant call, such as an insertion or a deletion within a threshold proximity (e.g., within 200 base pairs) of an initial variant call. Accordingly, the call integration system 106 may indicate a presence or absence of such an additional variant call using a code, such as a binary code of 0 for absent and 1 for present.

In some embodiments, the call integration system 106 further determines, as a read-based sequencing metric, an alignment of a contiguous sequence corresponding to the nucleotide reads with a reference sequence of a reference genome modified to include a variant corresponding to an initial genotype call. In particular, the call integration system 106 modifies the reference genome by changing nucleotide bases to reflect a variant, such as SNPs and indels in flanking regions. In theory, the modified reference genome may align perfectly with an alternate contiguous sequence, which provides some training benefit to a genotype-call-integration machine-learning model in accurately identifying variants.

To modify a reference genome to include a variant, the call integration system 106 can perform various steps. In particular, the call integration system 106 can remove a portion of a sequence corresponding to a deletion region (e.g., a deletion region for a deletion variant) from the reference genome. In some cases, the call integration system 106 replaces the relevant portion of the reference sequence in a FAST-All (FASTA) file with a contiguous sequence representing the relevant variant. The call integration system 106 can then regenerate the hash table using the modified FASTA file. In addition, the call integration system 106 can run mapping-and-alignment components of a call generation model on the modified reference genome. The call integration system 106 can further re-run variant caller components of the call generation model on the new mapping-and-alignment output.

For candidate variants where read-based evidence falls below a threshold (e.g., less than 5 or 10 nucleotide reads supporting a candidate variant call), one approach to finding missing reads is to modify the local reference sequence by replacing it with the contiguous sequence representing the candidate variant. For a true positive case, when reads are remapped with the modified reference genome, some of the nucleotide reads that were incorrectly mapped/aligned to the primary assembly of the reference genome would have a higher likelihood to be mapped correctly with a contiguous sequence representing the candidate variant and thereby increasing the read depth on the new modified reference genome. Based on the new mapping, if the call integration system 106 reruns the call generation model, the call generation model does not call a variant for a true homozygous deletion or an insertion for a true heterozygous deletion case. Additionally, the depth of read coverage should increase for the contiguous sequence representing the candidate variant relative to the original primary assembly, which should result in a more accurate variant call. The likelihood of achieving more accurate mapping could be estimated by aligning read length segments of the contiguous sequence representing the candidate variant to the reference genome.

In some embodiments, the call integration system 106 analyzes flanking regions of a variant (as called by the call generation model) within a sample sequence, where the flanking regions include base calls within a threshold proximity (e.g., within 200 base pairs) of the variant. For example, the call integration system 106 determines an initial variant based on an initial genotype call using a call generation model (e.g., a DRAGEN VC), modifies a reference genome to include a (portion of a) contiguous sequence that reflects the variant, and identifies flanking regions of a threshold size of 200 base pairs on either side of the variant. The call integration system 106 further analyzes the flanking regions (e.g., the left flank and the right flank) of the combined sequence to determine the presence or absence of variants. Indeed, the call integration system 106 can quantify the extent (e.g., the quantity, the magnitude, and/or the size) of single nucleotide polymorphisms (SNPs) and/or insertions or deletions (indels) based on a modified reference genome (e.g., the combined sequence of the reference genome and the contiguous sequence).

In some cases, the interpretation of a contiguous sequence is sensitive to scoring parameters and penalties within a Smith-Waterman algorithm. Accordingly, in these or other cases, the call integration system 106 measures sensitivity to Smith-Waterman scoring parameters/penalties using deletion counts from Concise Idiosyncratic Gapped Alignment Report (CIGAR) string outputs of multiple scoring parameter sets. The call integration system 106 can further use a maximum contiguous deletion length as well as the sum of all deletions corresponding to the genomic region spanned by the breakends as sequencing metrics (e.g., read-based sequencing metrics).

In some cases, the call integration system 106 determines a read-based sequencing metric in the form of a deletion length in nucleotide bases based on one or more soft clipped nucleotide reads. For instance, the call integration system 106 re-aligns soft clipped segments from nucleotide reads to determine a deletion length (or a length of a different type of variant). In some embodiments, the call integration system 106 re-aligns only soft clipped portions of reads to provide an estimate of a length of a deletion or some other variant. For example, the call integration system 106 performs re-alignment only if a size of a soft clipped portion satisfies (e.g., is greater than) a threshold number of soft clipped bases (e.g., 10 soft clipped bases or 20 soft clipped bases).

Additionally, in some embodiments, the call integration system 106 determines or computes a re-alignment offset for soft clipped segments (e.g., those that satisfy the length requirement) by: i) for soft clipped reads to the left of a called variant, aligning the soft clipped portion to the left of a current position/coordinate denoting the end of the soft clipping, ii) for soft clipped reads to the right of a called variant, aligning the soft clipped portion to the right of a current position/coordinate denoting the start of the soft clipping, iii) determining a distance in number of nucleotide bases between an aligned position/coordinate and a location of soft clipping from an original mapping, iv) determining a left mode and a right mode for all distances determined via steps i)-iii), and v) determining a left re-alignment offset and a right re-alignment offset by determining a difference between the left mode and deletion length determined by the call generation model (e.g., DRAGEN SV Caller) and between the right mode and the deletion length determined by the call generation model (e.g., DRAGEN SV Caller), such as a number of nucleotide bases determined from variant length—alt seq length.

Further, the call integration system 106 can determine a read-based sequencing metric in the form of a number of the nucleotide reads that exhibit a mapping quality metric that fails to satisfy a threshold mapping quality metric. To elaborate, the call integration system 106 corrects for cases where a true positive shows nucleotide reads with low MAPQ scores (i.e., below a threshold MAPQ) that are nevertheless correctly mapped (although local alignment may be incorrect). In some cases, the call integration system 106 utilizes MAPQ as a soft weighting to indicate likelihood of aligning with an alternate contiguous sequence or a reference genome. The call integration system 106 can further determine a count or a number of reads with mapping quality metrics (e.g., MAPQ scores) that fail to satisfy (or are below) a threshold mapping quality metric (e.g., MAPQ=10 or MAPQ=60 or a relative MAPQ threshold). In some cases, the call integration system 106 determines or generates a variant call based on the number of reads with low mapping quality metrics. In certain embodiments, such as in cases where MAPQ=60, the call integration system 106 further incorporates an XQ score to determine an extended range on the likelihood of a variant. The call integration system 106 can determine and incorporate a standard deviation of XQ across locally mapped reads for improved prediction of the genotype-call-integration machine-learning model.

As further noted above, in some embodiments, the call integration system 106 also determines an insert size representing a length of nucleotide-read fragments corresponding to an initial genotype call or variant call determined by the call generation model. Specifically, the call integration system 106 determines sizes or lengths (e.g., numbers of base pairs) for insertions (or other variants) within genomic region (e.g., an SV region) of a genomic sample.

In some cases, the call integration system 106 determines a read-based sequencing metric in the form of a palindrome metric. For instance, the call integration system 106 analyzes a portion of a reference sequence corresponding to a target genomic region where a variant is called (e.g., by a call generation model). Specifically, if the reference sequence in such a target genomic region is a palindrome (or within a threshold percentage of—or within a threshold number of base pairs from—a palindrome), then the likelihood of a folding effect increases. Based on the analysis, the call integration system 106 identifies or detects fragments or portions of a genomic sample (e.g., sub-sequences of reads) within a threshold distance (e.g., within 200 base pairs) from one another and that are palindromes (which can exhibit a deletion due to a folding effect during base calling). The call integration system 106 can determine or measure a distance or a closeness of (e.g., a number of base pairs separating) the segments of the palindrome metric. In some cases, the call integration system 106 further incorporates a permutation entropy with the palindrome metric such that a palindrome match (e.g., a pair of segments exhibiting a palindrome of each other) with higher permutation entropy increases a likelihood of a deletion (or some other variant).

Further, in some embodiments, the call integration system 106 determines a read-based sequencing metric in the form of a variant likelihood or probability representing a ratio of an initial variant call to a reference call for the one or more genomic coordinates based on an insert size. In particular, assuming there is no variant, then there is a certain implied insert size or fragment size. On the other hand, assuming there is a variant, then there is a different implied insert size or fragment size. Thus, based on a mean and a standard deviation of a fragment size, the call integration system 106 can determine which is more likely between a presence or absence of a variant. For instance, in some embodiments, the call integration system 106 determines a ratio of an initial variant call to a reference call for the one or more genomic coordinates according to the following formula:

$$\frac{{\prod }_{k = 0}^{{N}_{{A}^{- 1}}} {e}^{- \frac{{( {\tilde{l}}_{R , k} - {μ}_{I} )}^{2}}{2 {σ}_{I}^{2}}}}{{\prod }_{k = 0}^{{N}_{{A}^{- 1}}} {e}^{- \frac{{( {l}_{R , k} - {μ}_{I} )}^{2}}{2 {σ}_{I}^{2}}}}$$

where N_{A} is the number of reads showing evidence to support an alternate allele, l_{R,k} is the original estimated insert size corresponding to read k assuming no variant is present, \tilde{l}_{R,k} is the new estimated insert size based on alignment to the assembly of alternate contiguous sequences, μ_{I} is the mean insert size of a variant for the genomic sample, and σ_{I} is the standard deviation of the insert size of the variant for the genomic sample assuming a Gaussian distribution. In some cases, \tilde{l}_{R,k} is affected by the orientation of the split read and alignment relative to a candidate deletion (or another type of variant).

Depending on read orientation and alignment relative to a candidate variant genomic region, the call integration system 106 may subtract length of a proposed variant (e.g., deletion) from an original insert size estimate (e.g., based on reference mapping and alignment). When considering all nucleotide reads providing alternate allele supporting evidence, the call integration system 106 can determine the likelihood ratio (e.g., for alt vs. ref) based on projected insert sizes across the set of reads.

In some cases, the estimation of \tilde{l}_{R,k} is affected by the orientation of a split read serving as evidence for a variant (e.g., a deletion). Thus, the call integration system 106 adjusts insert size estimates based on read orientation (e.g., for forward and reverse cases). However, the contiguous sequence often will not match reference flanking regions. Thus, the insert sizes computation will depend on both read orientation and the start location of the split read relative to breakend after aligning with the contiguous sequence. Additionally, the reference starts (e.g., genomic coordinate for start of a variant) provided in a BAM file often do not include the soft clipped portions of the nucleotide reads, and because the insert size computation uses the actual start of the reads, the call integration system 106 adjusts reference starts to account for the amount of soft clipped bases.

In one or more embodiments, the call integration system 106 determines a read-based sequencing metric in the form of a confidence interval around ending breakpoints. In particular, the call integration system 106 utilizes the call generation model to determine a confidence interval as a measure of certainty of a breakpoint location. For example, the call integration system 106 determines a range of reference coordinates where a breakpoint might be located corresponding to a variant call. In some cases, the call integration system 106 determines the range of reference coordinates to reflect a threshold percentile (e.g., the 95th percentile) in terms of confidence interval.

In certain embodiments, the call integration system 106 further determines additional or alternative read-based sequencing metrics. For example, the call integration system 106 determines a homology length as a read-based sequencing metric. Specifically, the call integration system 106 determines a length of a nucleotide base sequence that is repeated in a target genomic region of a variant and/or a length of a nucleotide base sequence with at least a threshold measure of homology with other nucleotide base sequences (of similar lengths) within the target genomic region of the structural variant (e.g., HOMLEN=8 GCTTGAAC GCTTAAAC GCTAGAAC GCTTGAAC GCTTGTAC, etc.). In some cases, the call integration system 106 determines a length of an inserted nucleotide base sequence as a read-based sequencing metric. In these or other cases, the call integration system 106 determines a homology of an inserted nucleotide base sequence relative to a reference sequence within a target genomic region of a variant.

In one or more embodiments, the call integration system 106 determines additional or alternative read-based sequencing metrics, including: i) a comparative-mapping-quality-distribution metric indicating a mapping quality distribution comparing mapping qualities in relation to the reference sequence and mapping qualities in relation to alternative supporting reads, ii) a comparative-secondary-mapping-alignment metric indicating a comparison between secondary mapping in relation to bases in the reference sequence and bases in alternative supporting reads, iii) a comparative-mismatch-count metric indicating a comparison between mismatched nucleobases in relation to the reference sequence and mismatched bases in relation to alternative supporting reads, iv) a comparative-soft-clipping metric indicating a comparison between soft-clipping metrics in relation to the reference sequence and soft-clipping metrics in relation to alternative supporting reads, v) one or more comparative-read-depth metrics indicating comparisons between read depths of nucleotide reads and one or more average read depths (e.g., local average read depths at a particular genomic coordinate and global average read depths across a number genomic coordinates in a region), vi) one or more comparative-base-quality metric indicating comparisons between base qualities in relation to the reference sequence and base qualities in relation to alternative supporting reads (e.g., for overall base quality, early base quality, and late base quality in nucleotide reads), vii) a comparative-query-position metric indicating a comparison between query positions in relation to the reference sequence and query positions in relation to alternative supporting reads, viii) one or more contextual-information metrics indicating homopolymers and periodicity of nucleobase calls, ix) a strand-bias metric indicating a strand bias associated with one or more nucleotide reads, and x) a read-direction-bias metric indicating a read direction bias associated with the nucleotide reads.

B. Call-Model-Generated Sequencing Metrics

In addition to the read-based sequencing metrics 406a and 406b, as illustrated in FIG. 4B, the call integration system 106 generates call-model-generated sequencing metrics 412a and 412b. In particular, the call integration system 106 generates the call-model-generated sequencing metrics 412a and 412b from sequence data 408a and 408b, respectively, utilizing instances of a call generation model 410a and 410b. For example, the call integration system 106 extracts or determines sequence data 408a based on the read processing and mapping 404a described in relation to FIG. 4A. Similarly, the call integration system 106 extracts or determines the sequence data 408b based on the read processing and mapping 404b. In some cases, the call integration system 106 generates the sequence data 408a and 408b as part of one or more digital files, such as BCL and FASTQ files.

To generate such files, in some embodiments, the sequencing device 114 (or the call integration system 106) utilizes cluster generation and SBS chemistry to sequence millions or billions of clusters in a flow cell. During SBS chemistry, for each cluster, the sequencing device 114 (or the call integration system 106) stores nucleobase calls from the first type of nucleotide reads 402a and the second type of nucleotide reads 402b for every cycle of sequencing via real-time analysis (RTA) software. The sequencing device 114 (or the call integration system 106) utilizes RTA software to further store base call data in the form of individual base call data files (or BCLs). In some cases, the sequencing device 114 (or the call integration system 106) further converts the BCL files into sequence data 408a and 408b (e.g., via BCL to FASTQ conversion). For instance, the sequencing device 114 (or the call integration system 106) generates FASTQ files from the first type of nucleotide reads 402a and the second type of nucleotide reads 402b, where the FASTQ files includes sequence data 408a and 408b, respectively.

In some cases, the call integration system 106 generates the sequence data 408a and 408b for each cluster that passes an initial quality filter from a sample sequence. For example, the call integration system 106 generates entries for each cluster, where each entry includes four lines (or four items of sequence data): i) a sequence identifier with information about the sequencing run and the cluster, ii) nucleobase calls that make up the sequence (e.g., a sequence of A, C, T, G, and/or N calls), iii) a separator (e.g., a “+” sign), and iv) base-call-quality metrics indicating probabilities of correctness for the nucleobase calls (PHRED+33 encoded).

As further illustrated in FIG. 4B, the call integration system 106 implements, utilizes, or applies a call generation model 410a to process or analyze the sequence data 408a. Likewise, the call integration system 106 implements, utilizes, or applies a call generation model 410b to process or analyze the sequence data 408b. Indeed, in some embodiments, the call integration system 106 generates the call-model-generated sequencing metrics 412a and 412b by utilizing respective instances of the call generation model 410a and 410b to re-engineer raw sequencing metrics (e.g., raw sequencing metrics within the sequence data 408a and 408b). In particular, the instances of the call generation model 410a and 410b includes mapping-and-alignment components to map and align nucleobase calls from the sequence data 408a and 408b. In addition, the instances of the call generation model 410a and 410b includes variant calling components to generate initial genotype calls (e.g., reference-base calls such as nucleobase calls, variant calls, or non-variant calls) from the sequence data 408a and 408b. In some cases, the call integration system 106 extracts the call-model-generated sequencing metrics 412a and 412b that have been generated utilizing the mapping-and-alignment components and the variant calling components of the instances of the call generation model 410a and 410b.

To illustrate examples of the call-model-generated sequencing metrics 412a and 412b, in some cases, the call integration system 106 generates variant calling metrics including one or more of: i) genotype metrics corresponding to a GT field of a VCF file and indicating a genotype of a genomic coordinate, ii) a base-call-quality metric (e.g., DRAGEN QUAL score) indicating a quality score for genotype calls generated via the call generation model 410a or 410b, iii) genotype quality metrics (e.g., a GQ score) indicating a measure of confidence or quality of a predicted genotype for a genomic coordinate, iv) genotype probability metrics indicating one or more probabilities of various genotypes occurring at a genomic coordinate, v) PHRED-scaled-likelihood metrics or non-PHRED-scaled-likelihood metrics indicating probabilities of errors associated with genotype calls, vi) a call-model-generated-foreign-read-detection metric (e.g., foreign read detection (FRD) score) indicating a probability that one or more of the first type of nucleotide reads 402a or the second type of nucleotide reads 402b in a pileup might be foreign reads (e.g., their true location is elsewhere in the reference sequence), vii) a call-model-generated-base-quality-dropoff metric (e.g., base quality dropoff (BQD) score) indicating a probability of base quality dropoff based on one or more of strand bias, error position in a thread, or low mean base quality over a subset of the first type of nucleotide reads 402a and/or the second type of nucleotide reads 402b, viii) an average read depth ix) a normalized read depth, x) a read depth with mapq0 reads, xi) a read depth without mapq0 reads, xii) indel statistics (e.g., a polymerase chain reaction or “PCR” curve) and/or xiii) hidden Markov model (HMM) statistics, xiv) a secondary-alignment metric indicating a probability that a secondary genotype call is correct, xv) a base-context metric indicating contextual information for nucleotide around a genotype call, xvi) a nearby-call metric indicating nearby (e.g., adjacent or within a threshold degree of separation from) a genotype call, xvii) a joint-detection metric indicating a probability of detecting a joint corresponding to two or more overlapping genotype calls, and/or xviii) read-filtering metrics indicating threshold quality metrics or other metrics for filtering out genotype calls with low mapping quality, base quality, or other quality metrics, or others. The call integration system 106 generates the call-model-generated sequencing metrics 412a and 412b from internal (e.g., proprietary, and model-specific) variables that reflect interacting processing paths, corner cases, and difficult predictions/decisions.

As indicated above, in some cases, the call integration system 106 determines FRD scores according to the methods described in U.S. patent application Ser. No. 16/280,022 to Eric Jon Ojard, entitled System and Method for Correlated Error Event Mitigation for Variant Calling, filed Feb. 19, 2019, which is incorporated by reference herein in its entirety. In certain implementations, the call integration system 106 also (or alternatively) determines BQD scores, FRD scores, HMM statistics, and/or other variant calling metrics according to the methods described in U.S. patent application Ser. Nos. 17/165,828, 15/643,381, and 14/811,836, which are incorporated by reference herein in their entireties.

As illustrated in FIG. 4B, the call-model-generated sequencing metrics 412a and 412b include, but are not limited to, variant calling metrics extracted via the variant calling components of the instances of the call generation model 410a and 410b. In addition or in the alternative to the examples of the call-model-generated sequencing metrics 412a and 412b described above, in some cases, the call integration system 106 generates (e.g., via metric re-engineering) variant calling metrics including one or more of: i) a number of samples in a population, ii) a number of reads processed for generating genotype calls, a number of variants (e.g., SNPs and indels), iii) a number of biallelic sites (e.g., genomic coordinates that contain two observed alleles), iv) a number of multiallelic sites (e.g., a number of sites in a variant call file that contain three or more observed alleles), v) a number of SNPs, vi) numbers of different types of indels (e.g., homozygous insertions, heterozygous insertions, and heterozygous deletions), vii) a total number of heterozygous indels (e.g., insertion+deletion, insertion+SNP, or deletion+SNP), viii) a number of de novo SNPs (e.g., SNPs with de novo quality metrics that satisfy a threshold level), ix) a number of de novo indels (e.g., indels with de novo quality metrics that satisfy a threshold level), x) a number of de novo MNPs (e.g., MNPs with de novo quality metrics that satisfy a threshold level, xi) a number of SNPs in a first chromosome divided by a number of SNPs in a second chromosome, xii) a number of SNP transitions, xiii) a number of SNP transversions, xiv) a number of heterozygous variants, xv) a number of homozygous variants, xvi) a ratio between the number of heterozygous variants and the number of homozygous variants, xvii) a number of variants detected within a dbSNP reference file, and/or xviii) a total number of variants minus the number detected within the dbSNP file.

Additionally, the call-model-generated sequencing metrics 412a and 412b can include mapping-and-alignment sequencing metrics extracted via the mapping-and-alignment components of the call generation model 410a or 410b. For instance, the call integration system 106 generates or extracts (e.g., via metric re-engineering) mapping-and-alignment metrics including one or more of: i) a number of total input reads, ii) a number of duplicate marked reads, iii) a number of duplicate marked and mate reads removed, iv) a number of unique reads, v) a number of reads with mate sequenced, vi) a number of reads without mate sequenced, vii) indications of reads that fail quality checks, viii) indications of mapped reads, ix) a number of unique and mapped reads, x) a number of unmapped reads, xi) a number of singleton reads (e.g., where the read is mapped but the paired mate could not be read), xii) a number of paired reads, xiii) a number of properly paired reads (e.g., where both reads in a pair are mapped and fall within an acceptable range from each other based on an estimated insert length distribution), xiv) a number of discordant reads (e.g., not properly paired reads), xv) a number of paired reads mapped to different chromosomes, xvi) a number of paired reads mapped to different chromosomes that also have a mapping-quality metric of 10 or greater, xvii) percentages of reads within indels R1 and R2, xviii) percentages of bases in R1 and R2 that are soft clipped, xix) a number of mismatched bases in R1 and R2, xx) a number of bases with a base quality of at least 30 (e.g., total and/or in R1 or R2), xxi) a number of alignments (e.g., total alignments, secondary alignments, and/or supplementary alignments), xxii) an estimated read length, and xxiii) an estimated sample contamination.

C. Externally Sourced Sequencing Metrics

Turning now to FIG. 4C, the call integration system 106 generates, extracts, or determines externally sourced sequencing metrics 416. In particular, the call integration system 106 determines externally sourced sequencing metrics 416 from one or more databases external to the call integration system 106, such as a sequencing information database 414. For example, the call integration system 106 accesses sequencing metrics that are generic or applicable to sequencing nucleotides generally. In addition, the call integration system 106 accesses or determines sequencing information about a particular reference sequence (e.g., stored within the sequencing information database 414).

In some cases, the call integration system 106 determines externally sourced sequencing metrics 416 including: i) mappability metrics indicating an ease or difficulty of mapping a particular nucleotide sequence (or a particular nucleotide read or nucleobase call) to one or more genomic coordinates within a reference genome, ii) a guanine-cytosine-content metric indicating a count (or a dropout or a mean) of guanine-cytosine content in a reference nucleotide sequence (e.g., reference genome), iii) a replication-timing metric indicating a time required to replicate a particular number of nucleotides from a reference sequence, iv) one or more DNA-structure-metrics indicating DNA structures of a reference sequence (e.g., reference genome), v) a conservation metric indicating a measure of sequence conservation across multiple species (e.g., a measure of change relative to an average), vi) a confidence classification indicating a degree to which nucleobases at the one or more genomic coordinates can be accurately determined, vii) a repeat classification indicating a category of repetitive genomic region for the one or more genomic coordinates, viii) a cytosine quadruplex indicator indicating that one or more genomic coordinates are part of a cytosine quadruplex, ix) a guanine quadruplex indicator indicating that one or more genomic coordinates are part of a guanine quadruplex, and/or x) a homopolymer indicator indicating that one or more genomic coordinates are part of a homopolymer within a reference genome.

In some embodiments, the call integration system 106 determines the externally sourced sequencing metrics 416 by analyzing one or more genomic regions of a reference genome corresponding to (or aligning with) the one or more genomic coordinates for an initial genotype call. Many challenging variant calls occur in low complexity genomic regions of the reference genome. In some cases, these genomic regions are characterized by some combination of multiple instances of long repeat sequences (e.g., more than 50 base pairs), very high number (e.g., more than 10) of shorter repeat sequences (e.g., 4-8 repeated bases), and on occasion containing a subset of the bases (e.g. As and Ts but no Cs or Gs). The nucleotide reads that are aligned correctly to such low complexity genomic regions often have portions or fragments of the nucleotide reads that map to a more unique sequence flanking a repeat-heavy region. Alternatively, a reference genome or genomic sample may include some intermediate breaks (e.g., single bases in between the primary repeat pattern that breaks the repetitiveness) that help with alignment of nucleotide reads with a low complexity genomic region of a reference genome. However, when combined with SNPs, indels, and sequencing errors, the alignment and the collection of reads with sufficient evidence to compare reference versus alternate allele support becomes problematic. Thus, in some embodiments, the call integration system 106 monitors externally sourced sequencing metrics 416 (associated with complexity) which can be augmented with read-based sequencing metrics to provide an overall assessment of the likelihood of the presence of a variant (for both Bayesian and machine-learning approaches).

For example, the call integration system 106 accesses or determines sequencing information about a particular reference genome (e.g., stored within the sequencing information database 414). In some cases, the call integration system 106 determines externally sourced sequencing metrics 416 including a tandem repeat length in nucleotide bases of a target genomic region within a reference genome corresponding to a candidate region of a genomic sample. Specifically, the call integration system 106 analyzes portions of a reference genome that correspond to variant regions of a genomic sample to identify tandem repeats (e.g., sequences of two or bases that are repeated numerous times in a head-to-tail manner) and to further determine lengths (e.g., numbers of base pairs) within the tandem repeats.

In certain embodiments, the call integration system 106 determines an externally sourced sequencing metric in the form of a repetitiveness metric or homopolymer metric. Indeed, one indicator of a likelihood of a mis-mapping that needs to be corrected (e.g., a mis-mapping that results in a false positive) is based on repetitiveness of bases within a reference sequence. Thus, the call integration system 106 can utilize various sequencing metrics to measure this repetitiveness, including: i) a maximum repeat pattern length that indicates the maximum length of a sequence of bases that is repeated at least two times over the span of the (reference genome corresponding to the) candidate region, ii) a maximum repeat length percentage that indicates the percentage of the (portion of the reference genome corresponding to the) region that is consumed or occupied by the maximum repeat pattern length, and iii) a maximum homopolymer length that indicates the length of the longest sequence of the same base in the (portion of the reference genome corresponding to the) candidate region.

In addition or in the alternative to a repetitiveness metric, in some cases, the call integration system 106 determines an externally sourced sequencing metric in the form of a permutation entropy of nucleotide bases. For example, the call integration system 106 determines a measure of randomness of nucleotide sequences, which can be predictive of mapping/alignment accuracy. In some cases, the call integration system 106 determines a permutation entropy by determining an entropy over permutations of a nucleotide sequence of a given length. For instance, the call integration system 106 can determine permutation entropy according to the following formula:

S₁∈{A,C,G,T}

S₂∈{AA,AC,AG,AT,CA,CC,CG,CT,GA,GC,GG,GT,TA,TC,TG,TT}

S₃∈{AAA,AAC,AAG,AAT,ACT, . . . ,TTA,TTC,TTG,TTT}

S₄∈{AAAA,AAAC,AAAG,AAAT,AACA, . . . ,TTGT,TTTA,TTTC,TTTG,TTTT}

where S_{N} is a set of all permutations of length N base sequences, and where:

|S_{N}|=4^{N}

such that the probability of permutation element S_{N,k} occurring from set S_{N} is given by:

$${p}_{N , k} = \frac{{c}_{k}}{M - N + 1}$$

where cₖ is the number of occurrences of permutation element S_{N,k} in a sequence of length M. In some cases, the call integration system 106 normalizes the permutation entropy as:

$${E}_{N} = \frac{- {\sum }_{k ∈ K} {p}_{N , k} {log}_{2} {p}_{N , k}}{2 N}$$

where K⊆{0, . . . , 4^{N}−1} is the set of indices such that p_{N,k}>0.

As mentioned above, the call integration system 106 can further determine an externally sourced sequencing metric in the form of identifying a presence or absence of a cytosine quadruplex (C-quadruplex) or a guanine quadruplex (G-quadruplex) in a target genomic region. To elaborate, the call integration system 106 determines counts of cytosine calls and guanine calls within a target genomic region of a reference genome corresponding to a variant region of a genomic sample or genomic region under consideration for an initial variant call. To identify a cytosine quadruplex, the call integration system 106 identifies occurrences (within the target genomic region) of four or more instantiations of three consecutive cytosine bases separated by one or more different nucleotide bases (e.g., a pattern of CCC A CCC A CCC A CCC). Similarly, to identify a guanine quadruplex, the call integration system 106 identifies occurrences (within the target genomic region) of four or more instantiations of three consecutive guanine bases separated by one or more different nucleotide bases (e.g., a pattern of GGG T GGG T GGG T GGG).

In one or more embodiments, the call integration system 106 identifies a C-quadruplex or a G-quadruplex where up to a threshold number of nucleotide bases (e.g., up to 7 nucleotide bases) occur between instantiations of triple Cs or triple Gs. For instance, the call integration system 106 identifies GGG TACC GGG TGTACA GGG AAGTCT GGG as a G-quadruplex. In some cases, G-quadruplexes (and C-quadruplexes) are known to cause issues with sequencing. Accordingly, the call integration system 106 uses the presence of such sequences to adjust the confidence in the mapping and alignment of reads and the accuracy of subsequent contiguous sequence construction.

In certain embodiments, the call integration system 106 determines a data compression metric as part of the externally sourced sequencing metrics 416. In particular, the call integration system 106 determines a data compression metric that quantifies a measure of randomness of a sequence using one or more data compression algorithms. One such data compression algorithm for lossless compression is the Liv-Zempel-Welch algorithm. Using this algorithm, the call integration system 106 builds a dictionary of unique k-mers starting with length of one and comes up with an encoding for each entry in the dictionary. The call integration system 106 can utilize the number of keys in the dictionary for the structural variant and the flanking regions in the reference genome as a sequencing metric.

In addition or in the alternative to the externally sourced sequencing metrics 416 noted above, in some embodiments, the call integration system 106 determines a structural variant sequence alignment metric as part of the externally sourced sequencing metrics 416. For instance, the call integration system 106 uses gapless alignment scoring and Smith-Waterman alignment scoring of a proposed deletion sequence against the left/right flanking genomic regions in the reference. If there are multiple alignments that score above a threshold gapless alignment score and/or a threshold Smith-Waterman alignment score, the genotype-call-integration machine-learning model may process a variant sequence alignment metrics as an indicator that there is a higher likelihood of an imprecise variant call.

Further, the call integration system 106 can also determine a simulated read alignment metric as an externally sourced sequencing metric. Assuming that the contiguous sequence representing or including a variant is accurate, there should theoretically be many nucleotide reads with good alignment to the contiguous sequence, even for heterozygous deletions. However, for low evidence true-positive cases of variants, there is a likelihood of missing reads because the reads corresponding to the SV region were either mapped elsewhere or unmapped. The call integration system 106 can thus determine a likelihood of missing reads by simulating reads.

Specifically, the call integration system 106 chooses segments from the contiguous sequence equal in length to the SBS reads. The call integration system 106 chooses segments of the contiguous sequence that cross the breakend(s), that are equivalent to SBS read length, and that are aligned to the reference sequence in the SV region. For cases where alignment is ambiguous, alternate alignment scores will be higher and can serve as a possible guide for expected read depth. The call integration system 106 can further use the segment of the contiguous sequence equivalent to read length that is symmetric about the breakend to obtain the highest alignment scores. The call integration system 106 can further determine additional offsets from this symmetric point to check alternate alignment scores for a range of overlaps.

In one or more embodiments, the call integration system 106 determines additional or alternative sequencing metrics, including read-based sequencing metrics, call-model-generated sequencing metrics, and/or externally sourced sequencing metrics. For example, the call integration system 106 determines the sequencing metrics in following table, where each of the metrics belongs to one or more of the read-based sequencing metrics, call-model-generated sequencing metrics, and/or externally sourced sequencing metrics.

| Sequencing Metric | Description |
|---|---|
| Mappability | Lookup files of mappability scores by genomic |
| | position |
| Variant type | SNP or indel |
| Length | + for insertion, − for deletion, 0 for SNP |
| Indel_class_ref | For multiallelic positions, allows +/− |
| Indel_class_alt | For multiallelic positions, allows +/− |
| Ref_softclip | Number of softclips in reference-supporting |
| | reads |
| Alt_softclip | Number of softclips in alternate-supporting reads |
| Querypos_p | Statistical test of query position difference |
| | between reference-supporting reads and |
| | alternate-supporting reads |
| Leftpos_p | Statistical test of leftmost read position |
| | difference between reference-supporting reads |
| | and alternate-supporting reads |
| Seqpos_p | Statistical test of sequencing position difference |
| | between reference-supporting reads and |
| | alternate-supporting reads |
| Mapq_p | Statistical test of mapping quality difference |
| | between reference-supporting reads and |
| | alternate-supporting reads |
| Baseq_p | Statistical test of base quality difference between |
| | reference-supporting reads and alternate- |
| | supporting reads |
| Ref_baseq | Base quality of reference-supporting reads |
| Alt_baseq | Base quality of alternate-supporting reads |
| Context | Integer field capturing a five-base context |
| | around variant position |
| Major_mismatches_mean | Mean number of mismatches in reference- |
| | supporting reads |
| Minor_mismatches_mean | Mean number of mismatches in alternate- |
| | supporting reads |
| Mismatches_p | Statistical test of mismatch difference between |
| | reference-supporting reads and alternate- |
| | supporting reads |
| AF | Alternate allele frequency |
| AF_other | Allele frequency for any other allele |
| Dp | Sequence depth at position |
| AF_without_mapq0 | Alternate allele frequency after removing mapq0 |
| | reads (e.g., reads where MAPQ = 0) |
| Dp_without_mapq0 | Depth at position after removing mapq0 reads |
| Mapq_p_without_mapq0 | Statistical test of mapping quality difference |
| | between reference-supporting reads and |
| | alternate-supporting reads after removing mapq0 |
| | reads |
| Mosaic_likelihood | Calculated likelihood of a genetic mosaicism |
| Het_likelihood | Calculated likelihood of a heterozygous |
| | genotype |
| Refhom_likelihood | Calculated likelihood of a homozygous reference |
| | genotype |
| Althom_likelihood | Calculated likelihood of a homozygous alternate |
| | genotype |
| Mapq_difference | Difference in average MAPQ between reference- |
| | supporting reads and alternate-supporting reads |
| Mapq_ref | Average MAPQ on reference-supporting reads |
| Mapq_alt | Average MAPQ on alternate-supporting reads |
| Mapq_difference_without_mapq0 | Difference in average MAPQ between reference- |
| | supporting reads and alternate-supporting reads |
| | after removing mapq0 reads |
| Mapq_ref_without_mapq0 | Average MAPQ on reference-supporting reads |
| | after removing mapq0 reads |
| Mapq_alt_without_mapq0 | Average MAPQ on alternate-supporting reads |
| | after removing mapq0 reads |
| Encoded_position | Normalized position along chromosome |
| Qual | VC quality score |
| Gt | VC-derived genotype |
| Gq | VC-derived genotype quality score |
| Gerp200, gerp1000, gerp10000 | Gerp scores in window around variant position |
| Gc20, gc50, gc100, gc250, gc500, gc1000, | GC bias in window around variant position |
| gc2500, gc5000, gc10000, gc25000, gc75000 | |
| LowMap | Low mappability region flag |
| Homopolymer_100 | Window around known homopolymers in |
| | reference genome |
| Replication_timing | Score for replication timing at variant position |
| Ref_base | Encoded reference base |
| Alt_base | Encoded alternate base |
| Transition | Flag for a transition variant |
| FS | Fisher strand bias metric |
| ReadPosRankSum | Evidence of bias in position of alleles within |
| | reads that support them, between reference and |
| | alternate alleles |
| SOR | Strand bias |
| Max_depth | Maximum depth in active region |
| Avg_depth | Average depth in active region |
| Repeat_period | Repeat period at variant position |
| Repeat_length | Repeat length at variant position |
| Ins_gop | Insert gap opening penalty estimated at variant |
| | position |
| Del_gop | Delete gap opening penalty estimated at variant |
| | position |
| Is_columnwise_event | Flags variants that come from a simplified |
| | columnwise caller instead of full HMM-based |
| | calling |
| Base counts in window around variant position | Number of base counts within a window around |
| | a variant position |
| Ratio of soft clipping | Ref_softclip/alt_softclip |
| Insert size | Size of insertion |
| Population statistics | Population genotyping statistics |
| Dinuc flag | Flags dinucleotides, such as CPGs at variant |
| | position |
| Rosetta scores | Measure of repeatability of calls at locations or |
| | variant positions |
| RepeatMasker class | Indication of class within RepeatMasker |
| | database |
| g-quad flag | Indicates presence of g-quadruplex |
| Larger context window | Integer field capturing a context size (e.g., ten or |
| | twenty bases) around a variant position |

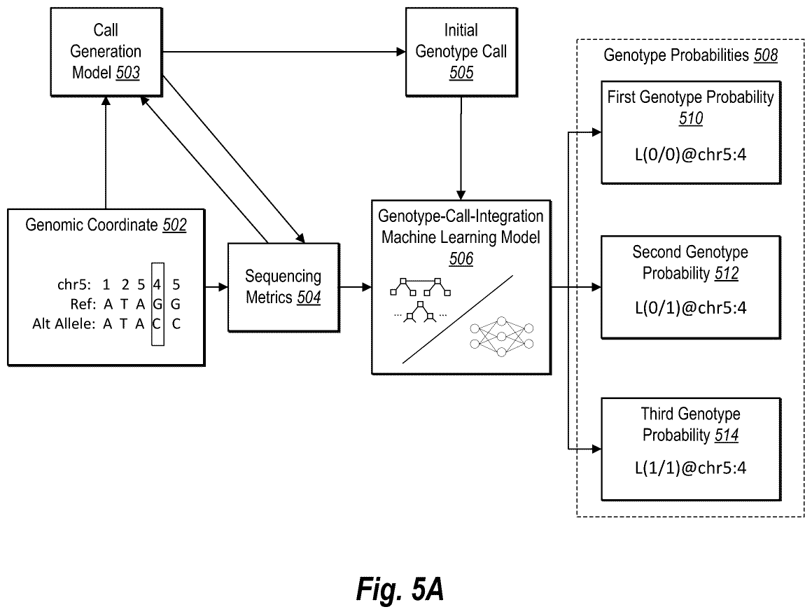

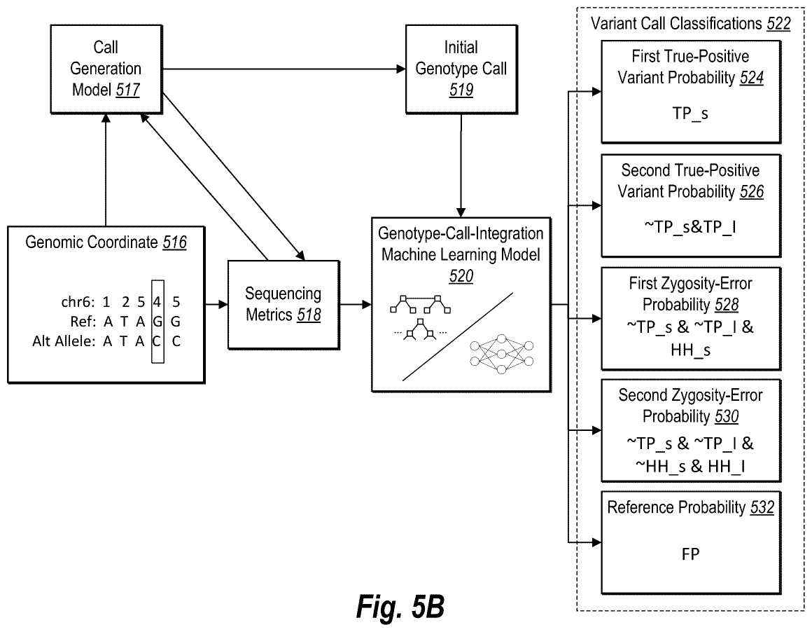

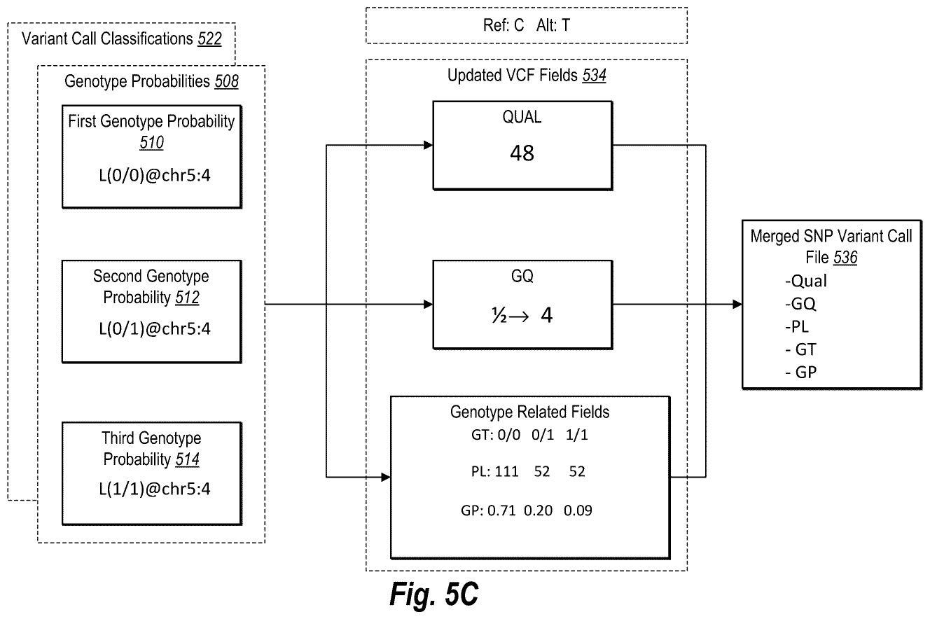

As mentioned above, in certain described embodiments, the call integration system 106 generates sets of machine learning predictions for different variant types using the sequencing metrics described above. In particular, the call integration system 106 utilizes a genotype-call-integration machine-learning model to generate genotype probabilities (for SNPs) or variant call classifications (for indels) corresponding to various genomic coordinates. In addition, the call integration system 106 determines an output genotype call by generating a variant call file (e.g., a merged variant call file) based on the genotype probabilities and/or the variant call classifications. In accordance with one or more embodiments, FIG. 5A-5C illustrate the call integration system 106 generating one or both of genotype probabilities and variant call classifications, generating a genotype call based on such likelihoods and/or classifications, and generating a merged variant call file comprising the genotype call based on such likelihoods and/or classifications. For example, FIG. 5A illustrates the call integration system 106 using a genotype-call-integration machine-learning model to generate genotype probabilities for (biallelic) SNPs based on sequencing metrics corresponding to initial genotype calls from different read types in accordance with one or more embodiments. FIG. 5B illustrates the call integration system 106 using a genotype-call-integration machine-learning model to generate variant call classifications for indels (or multiallelic SNPs or variant types other than biallelic SNPs) based on sequencing metrics corresponding to initial genotype calls from different read types in accordance with one or more embodiments. Thereafter, FIG. 5C illustrates the call integration system 106 generating a variant call file comprising output genotype calls based on the genotype probabilities and/or the variant call classifications in accordance with one or more embodiments.

As illustrated in FIG. 5A, the call integration system 106 identifies a genomic coordinate 502. For instance, the call integration system 106 identifies the genomic coordinate 502 from nucleobase calls corresponding to a sample nucleotide sequence or based on haplotype data corresponding to the genomic coordinate 502. In some cases, the call integration system 106 identifies the genomic coordinate 502 by determining (i) one or more nucleobase calls from nucleotide reads covering a genomic coordinate and (ii) that the one or more nucleobase calls satisfy one or more threshold sequencing metrics (e.g., a base-call-quality metric of Q30). Additionally or alternatively, in certain embodiments, the call integration system 106 identifies the genomic coordinate 502 by from a database comprising a haplotype reference panel correlated with specific genomic coordinates. Regardless of the identification method, in some cases, the call integration system 106 uses a call generation model 503 (e.g., a variant caller as part of a call generation model) to identify the genomic coordinate 502.

As depicted in FIG. 5A, the call integration system 106 also utilizes the call generation model 503 to generate an initial genotype call 505. To elaborate, the call integration system 106 utilizes the call generation model 503 (e.g., a DRAGEN caller) to generate the initial genotype call 505 to predict presence (or absence) of a variant (or a particular genotype) at the genomic coordinate 502. As described, the call generation model 503 generates the initial genotype call 505 by analyzing or processing sequencing metrics 504 (or a subset of the sequencing metrics 504, such as read-based sequencing metrics and externally sourced sequencing metrics). In addition, the call generation model 503 also generates some of the sequencing metrics 504 (e.g., the call-model-generated sequencing metrics) as part of predicting the initial genotype call 505.

Indeed, the call integration system 106 determines sequencing metrics 504 for the genomic coordinate 502. In particular, the call integration system 106 determines sequencing metrics associated with nucleotide reads, generated by the call generation model 503, or retrieved from an external source, as described above. Based on the sequencing metrics 504, the call integration system 106 further generates genotype probabilities 508 that together can indicate a measure of confidence or a probability that the genomic coordinate 502 includes or exhibits a SNP variant.

Specifically, as shown in FIG. 5A, the call integration system 106 utilizes a genotype-call-integration machine-learning model 506 to generate the genotype probabilities 508. For example, the genotype-call-integration machine-learning model 506 analyzes or processes the sequencing metrics 504 and the initial genotype call 505 as inputs to generate, as outputs, the genotype probabilities 508, including: i) a first genotype probability 510 that the initial genotype call 505 is a homozygous reference genotype at the genomic coordinate 502 (e.g., “L(0/0)@chr5:4”), ii) a second genotype probability 512 that the initial genotype call 505 is a heterozygous variant genotype at the genomic coordinate 502 (e.g., “L(0/1)@chr5:4”), and iii) a third genotype probability 514 that the initial genotype call 505 is a homozygous variant genotype at the genomic coordinate 502 (e.g., “L(1/1)@chr5:4”).

As mentioned, the call integration system 106 generates the genotype probabilities 508 to predict whether an SNP occurs at the genomic coordinate 502. To predict whether an indel occurs at a genomic coordinate, however, the call integration system 106 generates a different set of machine learning predictions. Specifically, the call integration system 106 generates variant call classifications that indicate presence (or absence) of an indel (or a multiallelic SNP or another variant type other than a biallelic SNP) at a genomic coordinate of a sample sequence.

As shown in FIG. 5B, the call integration system 106 utilizes a genotype-call-integration machine-learning model 520 to generate variant call classifications 522. To elaborate, the call integration system 106 utilizes genotype-call-integration machine-learning model 520 to generate the variant call classifications 522 based on sequencing metrics 518 and an initial genotype call 519 associated with a genomic coordinate 516. Indeed, similar to the discussion above regarding generating genotype probabilities for a biallelic SNP, the call integration system 106 likewise determines sequencing metrics 518 associated with the genomic coordinate 516, including read-based sequencing metrics, call-model-generated sequencing metrics, and externally sourced sequencing metrics. For instance, the call integration system 106 utilizes the call generation model 517 to analyze a subset of the sequencing metrics 518 (e.g., read-based sequencing metrics and/or externally sourced sequencing metrics) for determining the initial genotype call 519 (e.g., indicating a particular genotype or variant at the genomic coordinate 516). In some cases, the call generation model 517 further generates a subset of the sequencing metrics 518 (e.g., call-model-generated sequencing metrics) associated with the genomic coordinate 516.

In generating the variant call classifications 522 for the genomic coordinate 516, the call integration system 106 utilizes the genotype-call-integration machine-learning model 520. Particularly, the call integration system 106 utilizes the genotype-call-integration machine-learning model 520 to generate: i) a first true-positive variant probability 524 indicating a likelihood that the initial genotype call 519 (or an initial VCF file) from a first type of nucleotide reads (e.g., SBS reads) is a true positive at the genomic coordinate 516, ii) a second true-positive variant probability 526 indicating a likelihood that the initial genotype call 519 (or an initial VCF file) from a second type of nucleotide reads (e.g., assembled nucleotide reads) is a true positive at the genomic coordinate 516, iii) a first zygosity-error probability 528 indicating a likelihood that the initial genotype call 519 (or an initial VCF file) from a first type of nucleotide reads exhibits a genotype-zygosity error at the genomic coordinate 516, iv) a second zygosity-error probability 530 indicating a likelihood that the initial genotype call 519 (or an initial VCF file) from a second type of nucleotide reads exhibits a genotype-zygosity error at the genomic coordinate 516, and v) a reference probability 532 indicating a likelihood that the initial genotype call 519 at the genomic coordinate 516 is a homozygous reference genotype (or a false positive). In some cases, the variant call classifications 522 are mutually exclusive.

As shown, the first true-positive variant probability 524 is represented by “TP_s.” The symbol “TP_s” represents the probability that an input (x) is a true positive variant in a first variant call file (e.g., SBS variant call file), where “TP_s” can be formulated as P(tp_s|x)), “s” stands a first type of nucleotide reads, such “short reads” or SBS reads in particular. In addition, the second true-positive variant probability 526 is represented by “TP_1.” The symbol “˜TP_s&TP_1” represents the probability that the input (x) is not true positive in the first variant call file (e.g., SBS variant call file) and is a true positive in the second variant call file (e.g., assembled nucleotide read variant call file), where “˜TP_s&TP_1” can be formulated as P(˜tp_s&p_1|x)) and where “1” stands for “long reads” or assembled nucleotide reads.

By contrast, the first zygosity-error probability 528 is represented by “HH_s.” The symbol ““˜TP_s&TP_1&HH_s” represents the probability that the input (x) is not a true positive in the first variant call file (e.g., SBS variant call file), is not a true positive in the second variant call file (e.g., assembled nucleotide read variant call file), and is a het-hom error in the first variant call file (e.g., SBS variant call file). Additionally, the second zygosity-error probability 530 is represented by “HH_1.” The symbol “˜TP_s&˜TP_1&˜HH_s&HH_1” represents the probability that the input (x) is not a true positive in the first variant call file (e.g., SBS variant call file), is not a true positive in the second variant call file (e.g., assembled nucleotide read variant call file), is not a het-hom error in the first variant call file (e.g., SBS variant call file), and is a het-hom error in the second variant call file (e.g., assembled nucleotide read variant call file). Further, the reference probability 532 is represented by “FP,” which indicates the probability that the input (x) is a false positive and can be formulated as P(fP|x)).

To elaborate on the first zygosity-error probability 528 and the second zygosity-error probability 530, the call integration system 106 determines probabilities that predicted genotypes (e.g., initial genotype calls for different read types) at the genomic coordinate 516 are incorrect genotypes (e.g., a genotype incorrectly identified by the call generation model 517) or include an incorrect allele. To elaborate, in some cases, the call integration system 106 determines, based on a first type of nucleotide reads or a second type of nucleotide reads, a probability that a zygosity error (e.g., a het/hom error) exists at the genomic coordinate 516—e.g., where the alternate base is correct but the genotype is wrong—or a probability that the nucleobase calls represent either the wrong genotype altogether or the wrong allele(s) in the initial genotype call 519. For example, when determining a probability that a zygosity error exists, the call integration system 106 determines a probability that an alternate base call represented as “1” is correct, but the genotype is incorrect, such as a probability of incorrectly determining a 0/1 genotype call (e.g., A/T) instead of a correct 1/1 genotype call (e.g., T/T) (or vice versa when the correct genotype call is 0/1).

By determining the first zygosity-error probability 528 and the second zygosity-error probability 530, the call integration system 106 can fix inaccuracies of existing sequencing systems where incorrect calls are often indels. In particular, the call integration system 106 can more accurately generate genotype calls for genomic coordinates corresponding to indels where existing sequencing systems would determine a genotype call represent an incorrect genotype that represents an incorrect allele resulting from a long inserted or deleted sequence.

As further illustrated in FIG. 5B, the call integration system 106 utilizes the genotype-call-integration machine-learning model 520 to generate the first true-positive variant probability 524 and the second true-positive variant probability 526. In particular, the call integration system 106 generates the first true-positive variant probability 524 from a first type of nucleotide reads (e.g., SBS reads) and generates the second true-positive variant probability 526 from a second type of nucleotide reads (e.g., assembled nucleotide reads). In some cases, a true-positive variant probabilities indicates a probability of a correct variant call genotype at the genomic coordinate 516. For example, the call integration system 106 generates a probability that the initial genotype call 519 for the genomic coordinate 516 is correct as determined by the call generation model 517.

Continuing to FIG. 5C, in some embodiments, the call integration system 106 utilizes the genotype probabilities 508 and/or the variant call classifications 522 to update one or more data fields or variant call file fields (“VCF” fields) associated with a variant call file. For example, the call integration system 106 generates a merged SNP variant call file 536 based on the genotype probabilities 508 and the variant call classifications 522. Indeed, in some cases, the call integration system 106 generates a single merged variant call file that combines data from the genotype probabilities 508 for SNPs and from the variant call classifications 522 for indels.

As shown, the call integration system 106 generates updated VCF fields 534 that indicate, or correspond to, updated sequencing metrics for an output genotype call. Specifically, the call integration system 106 generates one set of updated VCF fields for the genotype probabilities 508 and generates another set of updated VCF fields for the variant call classifications 522. For purposes of illustration, FIG. 5C shows a few example fields within the updated VCF fields 534 without separately depicting one set of updated VCF fields for the genotype probabilities 508 and another set of updated VCF fields for the variant call classifications 522. In some cases, the call integration system 106 modifies or updates only certain VCF fields and does not update others based on the genotype probabilities 508 and/or the variant call classifications 522.

In other cases, the call integration system 106 does not update VCF fields. When generating genotype calls, for instance, the call integration system 106 does not update certain fields, such as a genotype (GT) field, based on the genotype probabilities 508 and/or the variant call classifications 522. Indeed, in some cases, the call integration system 106 does not modify or update a GT field because there may not be enough information to determine a new or updated genotype at a genomic coordinate.

To illustrate one embodiment, FIG. 5C depicts the call integration system 106 generating the updated VCF fields 534 for a genotype (GT) of ½, where cytosine represents a reference base (shown as “Ref: C”) at a genomic coordinate for an allele corresponding to the reference genome, adenine represents a first alternate base (“Alt 1: A”) at the genomic coordinate for a different allele, and thymine represents a second alternate base (“Alt 2: T”) at the genomic coordinate for yet a different allele. But FIG. 5C merely depicts examples of a possible reference base and possible alternate bases at a genomic coordinate. The call integration system 106 can generate genotype probabilities 508 and variant call classifications 522 to modify corresponding metrics in VCF fields for various other reference bases and alternate bases at genomic coordinates.

As further illustrated in FIG. 5C, the call integration system 106 generates an updated base call quality (QUAL) field. More specifically, the call integration system 106 modifies or updates a base-call-quality metric based on the genotype probabilities 508 and/or the variant call classifications 522 to indicate an accuracy of a genotype call. As shown, the updated base call quality field indicates a QUAL score of 48 for a variant at the corresponding genomic coordinate. In this example, the updated base-call-quality metric (e.g., QUAL score of 48) represents a score for any type of variant at the corresponding genomic coordinate. In addition, the call integration system 106 generates a modified or updated genotype quality (GQ) field. For instance, based on the variant call classifications 522, the call integration system 106 generates a modified or updated genotype quality metric indicating a likelihood or a probability that a predicted genotype at a genomic coordinate is correct. As shown, for instance, the updated genotype quality field indicates a genotype quality metric for a genotype call with a heterozygous genotype (e.g., a GQ score of 4 for a genotype of ½) for a multiallelic genomic coordinate.

In one or more embodiments, the call integration system 106 further generates or updates genotype probability fields and (in some cases) uses the genotype probability fields to rank alleles. To elaborate, the call integration system 106 generates an updated GT field by ordering candidate genotype calls at a genomic coordinate according to respective probabilities of belonging at a multiallelic genomic coordinate. For example, the call integration system 106 determines probabilities associated with a plurality of genotypes where each diploid genotype is composed of a pair of alleles. As another example, the call integration system 106 determines relative probabilities associated with a plurality of alleles (e.g., from a reference genome, a first alternate allele, and a second alternate allele) of belonging at the genomic coordinate.

In some embodiments, the call integration system 106 also or alternatively generates metrics for a PHRED-scale Likelihood (PL) field as part of the updated VCF fields. For example, the call integration system 106 generates metrics for a PL field that can indicate genotypes, such as homozygous reference, heterozygous, and homozygous alternate genotypes (e.g., with PL field nomenclature 9/0/3, respectively).

In one or more embodiments, the call integration system 106 generates allele-specific probabilities or likelihoods based on a relative probability of a genotype call corresponding to an allele from a call generation model versus any other (non-reference) genotype identified by a genotype-call-integration machine-learning model. For instance, in some embodiments, the call integration system 106 indicates relative probability scores for each allele corresponding to respective genotype calls in PL fields indicating normalized PHRED-scale likelihoods for genotypes and/or genotype probability (GP) fields indicating log-scaled posterior genotype probabilities (e.g., log 10-scaled) of data (e.g., sequencing metrics) given a called genotype.

As motivation for modifying certain VCF fields for an SNP, in some cases, the call integration system 106 utilizes a genotype-call-integration machine-learning model to generate the genotype probabilities 508 (whose probabilities sum to 1). In particular, the genotype-call-integration machine-learning model may generate the first genotype probability 510 as 0.1, the second genotype probability 512 as 0.2, and the third genotype probability 514 as 0.7. Based on the genotype probabilities 508 in such an example, the call integration system 106 generates the updated genotype probability fields by updating GT fields, GP fields, and PL fields using a combination of information from the genotype-call-integration machine-learning model and the call generation model.

As further illustrated in FIG. 5C, the call integration system 106 updates PL fields for different genotypes (GT). According to the normalized scale of a PL score, a relatively lower score (e.g., PL 0) for a genotype represents a relatively higher likelihood of the genotype being present at a genomic coordinate; and a relatively higher score (e.g., PL 101) for the genotype represents a relatively lower likelihood of the genotype being present at the genomic coordinate. For example, the call integration system 106 determines a PL score of 111 for the 0/0 genotype, a PL score of 52 for the 0/1 genotype, and a PL score of 52 for the 1/1 genotype. Accordingly, in FIG. 5C, the PL score of 52 indicates genotypes with the highest likelihood or the selected genotype (e.g., the 0/1 and the 1/1 genotypes) and the PL score of 111 represents the lowest likelihood (e.g., a 0/0 genotype).

In some cases, the call integration system 106 generates the updated genotype probability fields as a ranking of a plurality of alleles identified via the call generation model (without utilizing a genotype-call-integration machine-learning model). In other cases, the call integration system 106 utilizes a specialized version of a genotype-call-integration machine-learning model that is trained to generate the updated genotype probabilities fields based on the genotype probabilities 508 and/or the variant call classifications 522.

As further illustrated in FIG. 5C, the call integration system 106 generates or updates a variant call file, such as a merged SNP variant call file 536. For example, the call integration system 106 generates the variant call file from the updated VCF fields 534 corresponding to the genotype probabilities 508 and the variant call classifications 522, respectively. Thus, the call integration system 106 generates the merged SNP variant call file 536 for an SNP genotype call based on the genotype probabilities 508 and/or the variant call classifications 522. Indeed, in some embodiments, the call integration system 106 generates a merged variant call file that merges data for SNPs and indels from both the genotype probabilities 508 and the variant call classifications 522.

As indicated by FIG. 5C, the call integration system 106 can generate the merged SNP variant call file 536 to include the updated VCF fields 534, including a base-call-quality metric, a genotype quality metric, and/or updated genotype probability fields. For instance, the call integration system 106 selects VCF fields from initial genotype calls generated by a call generation model, such as an initial genotype call for SBS reads and an initial genotype call for assembled nucleotide reads, to include within a merged variant call file. In some embodiments, however, the call integration system 106 does not select fields but instead generates new VCF fields for a merged variant call file by using a genotype-call-integration machine-learning model to process the genotype probabilities 508 and the variant call classifications 522.

As mentioned, in some cases, the call integration system 106 updates only certain fields while other fields, such as a genotype (GT) field remain unchanged. For instance, the call integration system 106 updates the genotype quality field and the based call quality field. For other data fields such as normalized PHRED-scale likelihoods (PL) for genotypes and posterior genotype probability (GP), the call integration system 106 either: (i) maintains the field as-is, (ii) removes the field, or (iii) updates fields to reflect GQ for the called genotype and Class 0 output 0/0. In some cases, the call integration system 106 maintains the relative probabilities of other genotypes with respect to the called genotype to ensure consistent updates and that the called genotype is highest. In certain embodiments, by updating only the values for 0/0 and ½, the call integration system 106 maintains distances of other genotypes from the called genotype. By updating only certain fields, the call integration system can more efficiently generate (merged) variant call files, without regenerating entirely new variant call files (as done by some prior systems) and/or updating every field (even those that are unchanged by new predictions).

Within (or as a result of generating) a merged variant call file, the call integration system 106 can include or update one or more output genotype calls (e.g., variant calls) associated with a genomic coordinate, as determined based on the updated VCF fields 534. Indeed, to generate an output genotype call, the call integration system 106 can predict nucleobases from candidate alleles at the genomic coordinate (e.g., according to their respective probabilities and metrics indicated by the merged variant call file). Thus, the call integration system 106 can generate an output SNP and/or an output indel call from the merged SNP variant call file 536.

Because the call integration system 106 generates genotype calls based on multiple read types in a single pipeline (e.g., combining data from each type of read), there are some circumstances where nucleotide reads of different types are in conflict. Indeed, in certain cases, an alternate read for a first type of nucleotide reads (e.g., SBS reads) and an alternate read for a second type of nucleotide reads (e.g., assembled nucleotide reads) may disagree, where the different read types indicate different nucleotide bases. In such circumstances, the call integration system 106 can utilize a machine learning model trained to determine which read data is more accurate between the different read types (e.g., by determining which alternate to select between SBS reads and assembled nucleotide reads). In some embodiments, the call integration system 106 resolves the conflict or disagreement between the different read types by automatically selecting alts indicated SBS over those indicated by assembled nucleotide reads (or other read types).

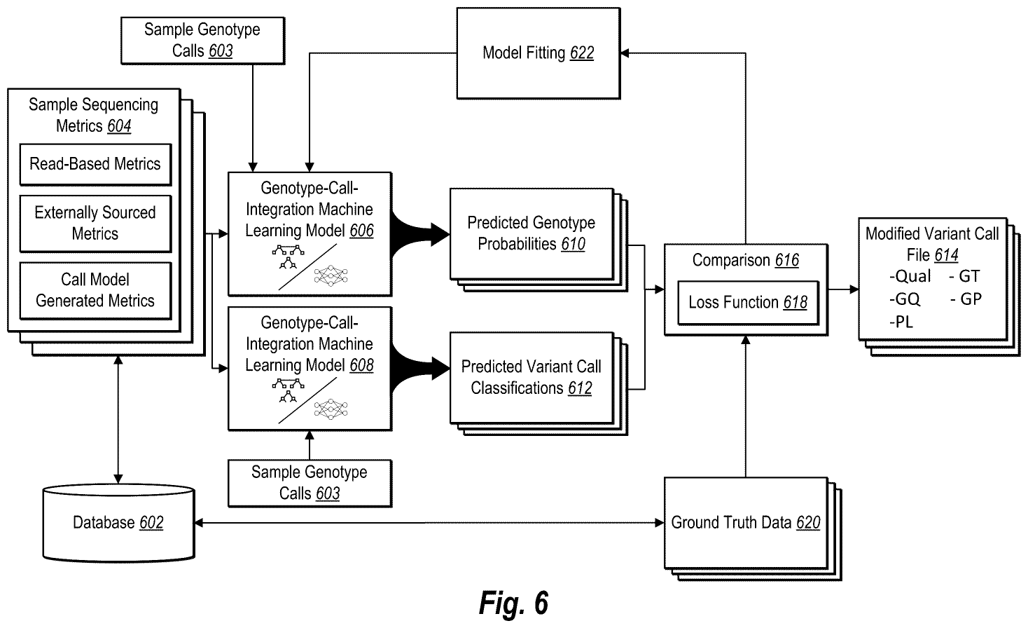

As mentioned above, in certain embodiments, the call integration system 106 trains or tunes a genotype-call-integration machine-learning model by learning model parameters, such as weights and biases for generating accurate genotype probabilities or accurate variant call classifications. In particular, the call integration system 106 utilizes an iterative training process to fit or train a genotype-call-integration machine-learning model by adjusting or adding decision trees or learning parameters that result in genotype probabilities (for SNPs) and/or variant call classifications (for indels). FIG. 6 illustrates the call integration system 106 training a genotype-call-integration machine-learning model in accordance with one or more embodiments. While FIG. 6 depicts different instances of a genotype-call-integration machine-learning model to succinctly illustrate a training process, in some embodiments, the call integration system 106 trains and adjusts model parameters for one instance or version of a genotype-call-integration machine-learning model and another instance or version of a genotype-call-integration machine-learning model 608 separately from one another. Accordingly, as depicted in FIG. 6, the call integration system 106 trains a genotype-call-integration machine-learning model 606 (e.g., a SNP-specific model) and a genotype-call-integration machine-learning model 608 (e.g., an indel-specific model) separately as different machine-learning models based on different ground truth data. Despite being trained as different machine-learning models, in some cases, the genotype-call-integration machine-learning model 606 and the genotype-call-integration machine-learning model 608 each comprise a same type of machine-learning model (e.g., gradient boosted decision trees, a deep learning transformer).

As illustrated in FIG. 6, the call integration system 106 trains one instance of the genotype-call-integration machine-learning model 606 to generate genotype probabilities for SNPs and trains another instance of a genotype-call-integration machine-learning model 608 to generate variant call classifications for indels. In particular, the call integration system 106 accesses sample sequencing metrics 604 from a database 602 to use as training data. For example, the call integration system 106 accesses sample sequencing metrics 604 including sample read-based metrics, sample externally sourced sequencing metrics, and sample call-model-generated sequencing metrics. In certain embodiments, the sample sequencing metrics 604 can be determined, generated, or derived from multiple different genomic samples analyzed or processed by different sequencing devices. Indeed, the call integration system 106 can train the genotype-call-integration machine-learning model 606 and/or the genotype-call-integration machine-learning model 608 using the sample sequencing metrics 604 with different dimensions of variability. Specifically, the sample sequencing metrics 604 can vary in the coverage or amount of sequencing performed on a sample to obtain the sequencing metrics. The sample sequencing metrics 604 can also (or alternatively) vary in library preparation method, sequencing device used to obtain the sample sequencing metrics 604, sequencing run quality (e.g., Q30, error rate, and/or % PF for percent passing filter).

In some cases, the sample sequencing metrics 604 have a corresponding ground truth variant call file (e.g., as part of ground truth data 620) associated with them (e.g., stored within the database 602), where the ground truth variant call file indicates actual VCF fields for an actual genotype call that result from the sample sequencing metrics 604. For instance, the call integration system 106 utilizes sample sequencing metrics 604 and the ground truth variant call file (e.g., as part of the ground truth data 620) from a training dataset generated by the United States Food and Drug Administration, called the PrecisionFDA dataset. In some cases, the sample sequencing metrics 604 include a subset of sample sequencing metrics for each genotype call in the ground truth variant call file. The ground truth variant call file can have a ground truth genotype call corresponding to the sample sequencing metrics.

As mentioned, the call integration system 106 trains a genotype-call-integration machine-learning model 606 for SNP genotype calls. To train the genotype-call-integration machine-learning model 606, the call integration system 106 inputs the sample sequencing metrics 604 and sample genotype calls 603 (e.g., initial genotype calls generated by a call generation model from the sample sequencing metrics 604) into the genotype-call-integration machine-learning model 606. In turn, the genotype-call-integration machine-learning model 606 generates predicted genotype probabilities 610 from the sample sequencing metrics 604. For instance, the genotype-call-integration machine-learning model 606 generates a predicted first genotype probability, a predicted second genotype probability, and a predicted third genotype probability, as described above.

As part of the training the genotype-call-integration machine-learning model 608 for indels, the call integration system 106 inputs the sample sequencing metrics 604 and the sample genotype calls 603 into the genotype-call-integration machine-learning model 608. In turn, the genotype-call-integration machine-learning model 608 generates predicted variant call classifications 612 based on the sample sequencing metrics 604. Specifically, in some embodiments, the genotype-call-integration machine-learning model 608 generates a set of five predicted variant call classifications, including a first true-positive variant probability, a second true-positive variant probability, a first zygosity-error probability, a second true-positive zygosity-error probability, and a reference probability, as described above.

Based on the predicted genotype probabilities 610 and/or the predicted variant call classifications 612, the call integration system 106 generates a modified variant call file 614. For instance, the call integration system 106 generates a modified variant call file from the predicted genotype probabilities 610 for training the genotype-call-integration machine-learning model 606. Additionally or alternatively, the call integration system 106 generates a modified variant call file from the predicted variant call classifications 612 for training the genotype-call-integration machine-learning model 608.

As further illustrated in FIG. 6, the call integration system 106 performs a comparison 616. Specifically, the call integration system 106 performs the comparison 616 to compare (i) the predicted genotype probabilities 610 with ground truth data 620 (e.g., ground truth genotype probabilities) and/or (ii) predicted variant call classifications 612 with ground truth data 620 (e.g., ground truth variant call classifications). In some embodiments, the call integration system 106 utilizes a loss function 618 to perform the comparison 616. For example, the call integration system 106 utilizes a cross entropy loss function to compare the predicted genotype probabilities 610 with ground truth genotype probabilities and/or the predicted variant call classifications 612 with the ground truth variant call classifications (e.g., to determine an error or a measure of loss between them). In cases where the genotype-call-integration machine-learning model 606 or 608 is an ensemble of gradient boosted trees, the call integration system 106 utilizes a mean squared error loss function (e.g., for regression) and/or a logarithmic loss function (e.g., for classification) as the loss function 618.

By contrast, in embodiments where the genotype-call-integration machine-learning model 606 is a neural network, the call integration system 106 can utilize a cross entropy loss function, an L1 loss function, or a mean squared error loss function as the loss function 618. For example, the call integration system 106 utilizes the loss function 618 to determine a difference between the predicted genotype probabilities 610 with ground truth genotype probabilities of the ground truth data 620 and/or the predicted variant call classifications 612 with the ground truth variant call classifications of the ground truth data 620.

In some embodiments, the call integration system 106 can utilize (i) a call generation model to generate an initial genotype call and (ii) the genotype-call-integration machine-learning model 606 or 608 to modify data fields corresponding to a variant call file for the initial genotype call—to generate a newly predicted genotype call. The call integration system 106 outputs such modified or recalibrated values as part of the modified variant call file 614. For example, the call integration system 106 determines recalibrated values for metrics within the modified variant call file 614, including a call-quality metric (QUAL), a genotype metric (GT), and a genotype-quality metric (GQ), among others.

As further illustrated in FIG. 6, the call integration system 106 performs model fitting 622. In particular, the call integration system 106 fits the genotype-call-integration machine-learning model 606 or 608 based on the comparison 616. For instance, the call integration system 106 performs modifications or adjustments to parameters (e.g., weights and biases) of the genotype-call-integration machine-learning model 606 or 608 to reduce the measure of loss from the loss function 618 and to use the adjusted parameters on a subsequent training iteration.

For gradient boosted trees, for example, the call integration system 106 trains the genotype-call-integration machine-learning model 606 or 608 on the gradients of the errors determined by the loss function 618. For instance, the call integration system 106 solves a convex optimization problem (e.g., of infinite dimensions) while regularizing the objective to avoid overfitting. In certain implementations, the call integration system 106 scales the gradients to emphasize corrections to under-represented classes (e.g., where there are significantly more true positives than false positive variant calls).

In some embodiments, the call integration system 106 adds a new weak learner (e.g., a new boosted tree) to the genotype-call-integration machine-learning model 606 or 608 for each successive training iteration as part of solving the optimization problem. For example, the call integration system 106 finds a feature (e.g., a sequencing metric) that minimizes a loss from the loss function 618 and either adds the feature to the current iteration's tree or starts to build a new tree with the feature.

In addition or in the alternative to gradient boosted decision trees, the call integration system 106 trains a logistic regression to learn parameters for generating genotype calls. To avoid overfitting, the call integration system 106 further regularizes based on hyperparameters such as the learning rate, stochastic gradient boosting, the number of trees, the tree-depth(s), complexity penalization, and/or L1/L2 regularization.

In embodiments where the genotype-call-integration machine-learning model 606 or 608 is a neural network, the call integration system 106 performs the model fitting 622 by modifying internal parameters (e.g., weights) of the genotype-call-integration machine-learning model 606 or 608 to reduce the measure of loss for the loss function 618. Indeed, the call integration system 106 modifies how the genotype-call-integration machine-learning model 606 or 608 analyzes and passes data between layers and neurons by modifying the internal network parameters. Thus, over multiple iterations, the call integration system 106 improves the accuracy of the genotype-call-integration machine-learning model 606 or 608.

Indeed, in some cases, the call integration system 106 repeats the training process illustrated in FIG. 6 for multiple iterations. For example, the call integration system 106 repeats the iterative training by selecting a new set of sequencing metrics for sample genotype calls, along with a corresponding ground variant call file. The call integration system 106 further generates a new set of predicted genotype probabilities and/or variant call classifications for each iteration along with a new modified variant call file. As described above, the call integration system 106 also compares genotype calls and/or data fields from the modified variant call file at each iteration with calls and/or data fields from the corresponding ground truth variant call file. The call integration system 106 further performs model fitting for each iteration as well. The call integration system 106 repeats this process until the genotype-call-integration machine-learning model 606 or 608 generates predicted genotype probabilities or variant call classifications that result in genotype calls or variant call files that satisfy a threshold measure of loss.

In some cases, the call integration system 106 uses a validation data set to determine when training is complete. For example, the call integration system 106 determines a loss on the validation data set (e.g., by comparing validation data with the predicted genotype probabilities 610 and/or the predicted variant call classifications 612). Based on determining that the loss value associated with the validation data set does not decrease (by a threshold amount) for at least a threshold number of iterations (e.g., 10 iterations), the call integration system 106 can determine that training is complete. In some embodiments, the call integration system 106 can perform training for a threshold number of iterations (e.g., 400 iterations), whereupon the call integration system 106 determines that training is complete.

Although not illustrated in FIG. 6, in certain embodiments, the call integration system 106 trains and adjusts model parameters for a single genotype-call-integration machine-learning model to generate different outputs (e.g., genotype probabilities and variant call classifications) in different training iterations or training epochs. For instance, the call integration system 106 (i) executes a set of training iterations to train and adjust model parameters for a genotype-call-integration machine-learning to generate genotype probabilities and (ii) executes another set of training iterations to train and adjust the same genotype-call-integration machine-learning model to generate variant call classifications. Because two different genotype-call-integration machine-learning models (e.g., an SNP-specific genotype-call-integration machine-learning model and an indel-specific genotype-call-integration machine-learning model) perform better in terms of recovering false positive variants and false negative variants, however, FIG. 6 depicts the genotype-call-integration machine-learning model 606 and/or the genotype-call-integration machine-learning model 608 being trained separately.

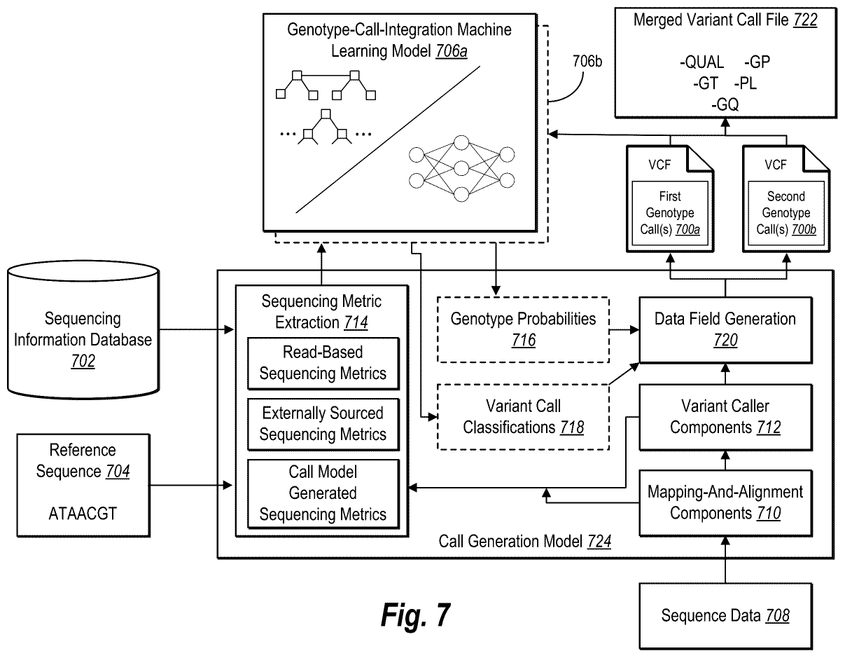

As mentioned, in certain described embodiments, the call integration system 106 utilizes a genotype-call-integration machine-learning model together with a call generation model to generate a genotype call. In particular, the call integration system 106 utilizes outputs of the genotype-call-integration machine-learning model to modify data fields corresponding to a variant call file comprising genotype call(s) initially generated by a call generation model. FIG. 7 illustrates the call integration system 106 generating genotype call(s) and modifying fields of a variant call file comprising the genotype call(s) and reported metrics based on outputs of a genotype-call-integration machine-learning model and a call generation model in accordance with one or more embodiments.

As illustrated in FIG. 7, the call integration system 106 accesses a sequencing information database 702, a reference sequence 704, and sequence data 708 extrapolated from one or more nucleotide reads (e.g., a first type of nucleotide reads and/or a second type of nucleotide reads). Indeed, the call integration system 106 performs sequencing-metric extraction 714 to extract or re-engineer sequencing metrics as described above. For example, the call integration system 106 generates read-based sequencing metrics, externally sourced sequencing metrics, and call-model-generated sequencing metrics. In some cases, the call integration system 106 utilizes mapping-and-alignment components 710 of a call generation model 724 to determine mapping-and-alignment sequencing metrics as described above. In addition, the call integration system 106 utilizes variant caller components 712 of the call generation model 724 to generate variant calling metrics as described above. Further, the call integration system 106 determines read-based sequencing metrics and externally source sequencing metrics as well (e.g., from the sequencing information database 702 and/or the reference sequence 704).

As further illustrated in FIG. 7, the call integration system 106 generates genotype probabilities 716 and/or variant call classifications 718. By analyzing the sequencing metrics, first genotype call(s) 700a corresponding to a first type of nucleotide reads, and second genotype call(s) 700b corresponding to a second type of nucleotide reads, the call integration system 106 utilizes a genotype-call-integration machine-learning model 706a to generate the genotype probabilities 716 for SNPs, as described herein. In addition, by analyzing the sequencing metrics, the first genotype call(s) 700a corresponding to a first type of nucleotide reads, and the second genotype call(s) 700b corresponding to a second type of nucleotide reads, the call integration system 106 utilizes a genotype-call-integration machine-learning model 706b to generate the variant call classifications 718 for indels, as described herein. As described above, the first genotype call(s) 700a corresponding to a first type of nucleotide reads and second genotype call(s) 700b corresponding to a second type of nucleotide reads can come from different read-type pipelines.

In some cases, the genotype-call-integration machine-learning model 706a or 706b is an ensemble of gradient boosted trees that processes the sequencing metrics to generate the genotype probabilities 716 or variant call classifications 718. For instance, the genotype-call-integration machine-learning model 706a or 706b includes a series of weak learners such as non-linear decision trees that are trained in a logistic regression to generate the genotype probabilities 716 or variant call classifications 718. In some cases, the genotype-call-integration machine-learning model 706a or 706b includes metrics within various trees that, based on the training described above, define how to process the sequencing metrics to generate the respective outputs.

As suggested above, in some embodiments, the call integration system 106 can utilize the genotype-call-integration machine-learning models 706a and 706b together. For example, the call integration system 106 utilizes the genotype-call-integration machine-learning models 706a and 706b to generate the genotype probabilities 716 and the variant call classifications 718, respectively. For example, the call integration system 106 utilizes two (or more) different genotype-call-integration machine-learning models in parallel, each trained with different random seeds (e.g., for different biases to process data differently) and/or on different training data for different types of variants, resulting in different predicted outputs.

In some embodiments, the call integration system 106 further generates a combined set of predictions from the outputs of the different genotype-call-integration machine-learning models 706a and 706b. For instance, the call integration system 106 combines (e.g., averages or totals) metrics from the genotype probabilities 716 and the variant call classifications 718. In some embodiments, the call integration system 106 determines a mean across predictions from different models and renormalizes the mean. In other embodiments, the call integration system 106 learns linear weights and adapts the weights to minimize overall error or loss. In still other embodiments, the call integration system 106 weights the genotype probabilities and/or the variant call classifications for respective genotype-call-integration machine-learning models based on the inverse of average error across the models.

In one or more implementations, the call integration system 106 further utilizes a metamodel subsequent to the genotype-call-integration machine-learning models 706a and 706b. For example, the call integration system 106 generates the genotype probabilities 716 (e.g., the genotype probabilities 508) and the variant call classifications 718 (e.g., the variant call classifications 522), as described above, and utilizes a classification-combiner-machine learning model to combine them. Specifically, the call integration system 106 can combine genotype probabilities and variant call classifications generated from each genotype-call-integration machine-learning model by selecting weights to apply to the variant call classifications generated by each genotype-call-integration machine-learning model. Indeed, in some cases, the call integration system 106 trains the classification-combiner-machine learning model to determine, select, or predict respective weights for genotype-call-integration machine-learning models to result in a highest accuracy or a minimized loss.

As an example of generating the genotype probabilities 716 and/or the variant call classifications 718, in some embodiments, the call integration system 106 utilizes statistics to summarize a mapping quality distribution of reference supporting reads and alternative supporting reads (e.g., for a comparative-mapping-quality-distribution metric). The call integration system 106 can determine and utilize the mean of the MAPQ for reads supporting an alternative allele from SBS reads and from assembled nucleotide reads. In these or other embodiments, the genotype-call-integration machine-learning model 706a or 706b learns from the data that, when the MAPQ of an alternative allele (indicated by SBS reads or assembled nucleotide reads) is low and a depth metric is high relative to other MAPQ and depth metrics in distributions, a resultant genotype call is more likely to be a false positive. Indeed, as the probability of a false positives increases, the MAPQ metrics would likely decrease.

As a further example, in some cases, the call integration system 106 compares a mapping quality (e.g., MAPQ) associated with an SBS read and/or an assembled nucleotide read with a mapping-quality threshold. For instance, the call integration system 106 utilizes a mapping-quality threshold such as a threshold difference between best and second-best alignment scores. Upon determining that one or more of mapping qualities for the different read types does not satisfy the threshold, the call integration system 106 adjusts one or more of the genotype probabilities 716 or variant call classifications 718 accordingly (e.g., to select a read with a higher MAPQ).

In addition (or in the alternative), the call integration system 106 can determine the genotype probabilities 716 and/or the variant call classifications 718 by utilizing an accumulation of statistical analyses over complex functions (depending on the architecture of the genotype-call-integration machine-learning model 706a or 706b) to determine how to best fit the data. For example, as described above, the call integration system 106 trains the genotype-call-integration machine-learning model 706a or 706b to minimize a loss generated from a number of (different types of) sequencing metrics to determine weights and biases that best fit the data (e.g., that result in a reduced or minimized loss).

As further illustrated in FIG. 7, in addition to generating the genotype probabilities 716 and the variant call classifications 718, the call integration system 106 performs data field generation 720. More specifically, the call integration system 106 generates data fields for one or more variant call files. In some cases, the call integration system 106 generates a first variant call file that includes the first genotype call(s) 700a and further generates a second variant call file that includes the second genotype call(s) 700b. As mentioned, the call integration system 106 can utilize the first genotype call(s) 700a and/or the second genotype call(s) 700b for generating predictions, such as the genotype probabilities 716 and the variant call classifications 718. As further shown, the call integration system 106 can use the data field generation 720 to generate a merged variant call file 722 (e.g., by combining all or selecting part of first and second variant call files) to indicate an output genotype call. To generate the merged variant call file 722, the call integration system 106 utilizes the variant caller components 712 of the call generation model 724 and modifies or maintains values for such data fields based the genotype probabilities 716 and/or the variant call classifications 718.

For instance, the call integration system 106 modifies various metrics such as quality metrics, mapping metrics, or other metrics associated with the genotype call. As mentioned, in some cases, the call integration system 106 selects metrics associated with a first or a second type of nucleotide reads and/or associated with the genotype probabilities 716 for SNPs and/or the variant call classifications 718 for indels. In other cases, the call integration system 106 generates new metrics from the data generated by the call generation model 724 and/or the genotype-call-integration machine-learning model 706a or 706b. In certain embodiments, the genotype call is represented or defined by the merged variant call file 722 which includes metrics corresponding to the data fields, such as a call-quality metric corresponding to a call-quality field, a genotype metric corresponding to a genotype field, and a genotype-quality metric corresponding to a genotype-quality field.

In certain embodiments, the call integration system 106 generates (data fields for) a genotype call utilizing the variant caller components 712 together with the genotype probabilities 716 and/or the variant call classifications 718. For instance, the call integration system 106 generates, for inclusion within the merged variant call file 722 and utilizing the variant caller components 712, data fields for various metrics of a genotype call such as nucleotide(s) included in the call, a call quality (QUAL), a genotype (GT), a genotype quality (GQ), one or more normalized PHRED-scale likelihoods (PL), and/or a genotype probability (GP).

In one or more embodiments, the call integration system 106 recalibrates or modifies a genotype call (or generates a new genotype call) using the genotype probabilities 716 from the genotype-call-integration machine-learning model 706a and/or the variant call classifications 718 from the genotype-call-integration machine-learning model 706b. As described, the call integration system 106 modifies the genotype call by modifying or recalibrating data fields for one or more of the metrics associated with the genotype call (e.g., as included within the merged variant call file 722).

To update or recalibrate the call-quality metric (QUAL) associated with a genotype call, for instance, the call integration system 106 determines how each of the genotype probabilities 716 and/or the variant call classifications 718 impact or affect the base-call-quality metric. For example, the call integration system 106 determines that a high probability for a genotype error results in a lower overall genotype quality and possibly a different overall call quality. As another example, the call integration system 106 determines that a high probability for a false positive variant results in a lower overall call quality. As yet another example, the call integration system 106 determines that a high probability for a true positive variant results in a higher overall (variant) call quality. The call integration system 106 accordingly updates the genotype along with the genotype quality and the call quality associated with the genotype call.

In one or more implementations, the call integration system 106 generates a combination (e.g., a weighted combination or an average) of the genotype probabilities 716 and/or the variant call classifications 718 to recalibrate the call-quality metric. In particular, the call integration system 106 weights the various predictions of the genotype probabilities 716 and/or the variant call classifications 718 according to their respective impact on (variant) call quality. In some cases, the call integration system 106 weights each genotype probability or variant call classification evenly, while in other cases the call integration system 106 determines different weights for each. In any event, the call integration system 106 determines a weighted combination or a weighted average of the genotype probabilities 716 and the variant call classifications 718 to recalibrate (increase or decrease) a call-quality metric for a genotype call (e.g., an initial variant call).

To update or recalibrate the genotype metric (e.g., within the GT field of the merged variant call file 722) associated with a genotype call, the call integration system 106 utilizes one or more of the genotype probabilities 716 and/or the variant call classifications 718. For example, the call integration system 106 compares the various constituent predictions of each to determine which of the genotype probabilities 716 or the variant call classifications 718 has a highest probability. In some cases, the call integration system 106 utilizes the genotype probability and/or the variant call classification with the highest probability to recalibrate the genotype metric (e.g., from 0 as corresponding to the reference base to 1 as corresponding to a first alternative supporting read).

To update or recalibrate the genotype-quality metric (e.g., within the GQ field of the merged variant call file 722) associated with a genotype call, the call integration system 106 utilizes one or more of the genotype probabilities 716 and/or variant call classifications 718. More specifically, the call integration system 106 determines how each of the genotype probabilities 716 and/or variant call classifications 718 affect the genotype-quality metric. The call integration system 106 recalibrates the genotype-quality metric accordingly (e.g., by increasing or decreasing the quality score between 0 to 10 or 0 to 100 or on some other scale). For example, the call integration system 106 determines that a higher genotype error probability (generally) indicates a lower genotype-quality metric, and the call integration system 106 reduces the metric accordingly.

In some cases, the call integration system 106 determines a combination (e.g., a weighted combination or a weighted average) of the genotype probabilities 716 and/or the variant call classifications 718 to modify the genotype-quality metric. For example, the call integration system 106 determines a combined effect that the genotype probabilities 716 and/or the variant call classifications 718 have on the genotype-quality metric. As another example, the call integration system 106 determines individual impacts that each constituent prediction of the genotype probabilities 716 and/or the variant call classifications 718 has on the genotype-quality metric and weights each accordingly. The call integration system 106 further recalibrates the genotype-quality metric by increasing or decreasing its value based on the indicated probabilities.

As described, the call integration system 106 generates an output genotype call from the same set of sequencing metrics (or a subset of the sequencing metrics that are shared between the genotype-call-integration machine-learning models 706a and 706b and the call generation model 724). Indeed, the call integration system 106 can operate the genotype-call-integration machine-learning model 706a or 706b in parallel with the call generation model 724 to generate metrics for an output genotype call, genotype probabilities 716, and variant call classifications 718 for recalibrating the generated metrics.

In one or more implementations, the call integration system 106 updates or otherwise modifies the data fields for the merged variant call file 722 according to particular algorithms. After modifying such data fields, the call integration system 106 can generate the merged variant call file 722 (e.g., a post-filter variant call file) to include metrics reflecting the updated data fields. For instance, in some cases, the call integration system 106 updates the QUAL field for every variant based on the probability of a false positive variant. As indicated above, in some cases, QUAL indicates the probability that there is some kind of variant (or other nucleobase call) at a given location, measured in PHRED scale.

As suggested above, in some embodiments, the call integration system 106 increases or decreases a base-call-quality metric (e.g., Q score) for a genotype call. Based on the genotype probabilities 716 and/or variant call classifications 718, for example, the call integration system 106 increases base-call-quality metrics for genotype calls that would not have previously passed a quality filter and determines that the increased base-call-quality metrics now passes the quality filter. In some such cases, the call integration system 106 includes genotype calls with such increased base-call-quality metrics (passing the quality filter) in a post-filter variant call file. By contrast, in other cases, the call integration system 106 decreases base-call-quality metrics for genotype calls that previously would have passed a quality filter and determines that the decreased base-call-quality metrics now fail the quality filter. In some such cases, the call integration system 106 excludes genotype calls with decreased base-call-quality metrics (failing the quality filter) from a post-filter variant call file, but includes the genotype calls with such decreased base-call-quality metrics in a pre-filter variant call file.

For example, the call integration system 106 can remove false positive variant calls and recover false negative variant calls by changing corresponding base-call-quality metrics. To remove a false positive, in some cases, the call integration system 106 decreases the base-call-quality metric of a genotype call that initially passed a quality filter-based on the genotype probabilities 716 and/or variant call classifications 718 from the genotype-call-integration machine-learning models 706a and 706b. Based on determining the decreased base-call-quality metric falls below a threshold metric (e.g., a Q score of 3.0 or 10.0), the call integration system 106 determines that the genotype call no longer passes the quality filter. The call integration system 106 thus filters out, or removes, the false positive-genotype call that initially passed the filter by changing its base-call-quality metric.

In addition to removing false positive variant calls based on changes to base-call-quality metrics, the call integration system 106 can remove false positive variant calls based on changes to genotype. To remove a false positive, in some cases, the call integration system 106 changes a genotype of an initial genotype call indicating a different nucleobase than a reference base (e.g., GT=1 or 2) to a genotype of an updated genotype call indicating a same nucleobase as the reference base (e.g., GT=0). Based on the genotype being the same as the reference base, the call integration system 106 does not identify the genotype call as a variant and, in some cases, excludes data for the genotype call from the merged variant call file 722. For instance, the call integration system 106 can use a null-data indicator for a genotype call (or a particular field) of the merged variant call file 722. In some cases, the call integration system 106 uses a null-data indicator in cases where a certain sequencing metric does not apply to a particular variant call or VCF field (e.g., where SBS-based calls use different metrics than assembled-nucleotide-read-based calls).

In generating the merged variant call file 722, in some embodiments, the call integration system 106 determines a first pipeline-accuracy likelihood for a first pipeline (e.g., based on a first read type) and a second pipeline-accuracy likelihood for a second pipeline (e.g., based on a second read type). To elaborate, the call integration system 106 determines a first pipeline-accuracy likelihood of a first genotype call (e.g., a genotype call generated based on SBS reads) being more accurate than a second genotype call (e.g., a genotype call generated based on assembled nucleotide reads). The call integration system 106 also determines a second pipeline-accuracy likelihood of the second genotype call being more accurate than the first genotype call. Indeed, the call integration system 106 can determine, using the genotype-call-integration machine-learning model 706a and/or 706b, a likelihood or a probability a first genotype call and/or a second genotype call is more accurate. Based on the pipeline-accuracy likelihood(s), the call integration system 106 can also generate an output genotype call (and corresponding fields within the merged variant call file 722) from the first genotype call and/or the second genotype call.

To recover a false negative, the call integration system 106 increases the base-call-quality metric of a genotype call that initially failed a quality filter. Based on determining the increased base-call-quality metric exceeds a threshold metric, the call integration system 106 determines that the genotype call passes the quality filter. The call integration system 106 thus recovers a false-negative-genotype call that was initially filtered out by changing its base-call-quality metric.

In addition to recovering false negative variant calls based on changes to base-call-quality metrics, the call integration system 106 can recover false negative variant calls based on changes to genotype. To recover a false negative, in some cases, the call integration system 106 changes a genotype of an initial genotype call indicating the same nucleobase as a reference base (e.g., GT=0) to a different genotype of an updated genotype call indicating a different nucleobase than the reference base (e.g., GT=1 or 2). Based on the differing genotype of the updated genotype call and a passing base-call-quality metric, the call integration system 106 identifies the genotype call as a variant and includes the genotype call within the merged variant call file 722.

Indeed, in some implementations, the call integration system 106 operates in a specific sequential order utilizing the call generation model 724 and the genotype-call-integration machine-learning models 706a and 706b. For example, the call integration system 106 generates a FASTQ file by converting a BCL file to FASTQ. In addition, the call integration system 106 (subsequently) utilizes the mapping-and-alignment components 710 of the call generation model 724 to map and align nucleobases from a sample nucleotide sequence. In some cases, the call integration system 106 maps and aligns the nucleobases of the sample sequence in relation to the reference sequence 704 (e.g., reference genome) and/or various alternative supporting reads.

After mapping and aligning, as described herein, the call integration system 106 then utilizes the variant caller components 712 of the call generation model 724 to generate an initial genotype call for the sample sequence corresponding to a particular genomic coordinate-based on various sequencing metrics. After or at the same time, the call integration system 106 also applies the genotype-call-integration machine-learning models 706a and 706b to generate the genotype probabilities 716 and the variant call classifications 718 from sequencing metrics extracted via the mapping and aligning, the variant calling, and/or from other sources as described above. Based on the genotype probabilities 716 and/or the variant call classifications 718, the call integration system 106 recalibrates the genotype call (e.g., by modifying various data fields corresponding to specific metrics of the nucleobase call such as QUAL, GT, GQ, GP, and/or PL), as described above.

In some cases, the call integration system 106 further applies a quality filter to the genotype call to determine whether the genotype call passes the quality filter (e.g., a hard pass filter of Q20 or other Q score). The call integration system 106 subsequently identifies a subset of genotype calls that represent variants from reference bases and pass the quality filter. The call integration system 106 further generates a modified or updated variant call file (e.g., the merged variant call file 722) that includes the subset of genotype calls and recalibrated metrics for the subset of genotype calls, such as updated QUAL metrics, updated GT metrics, updated GQ metrics, updated GP metrics, and/or updated PL metrics.

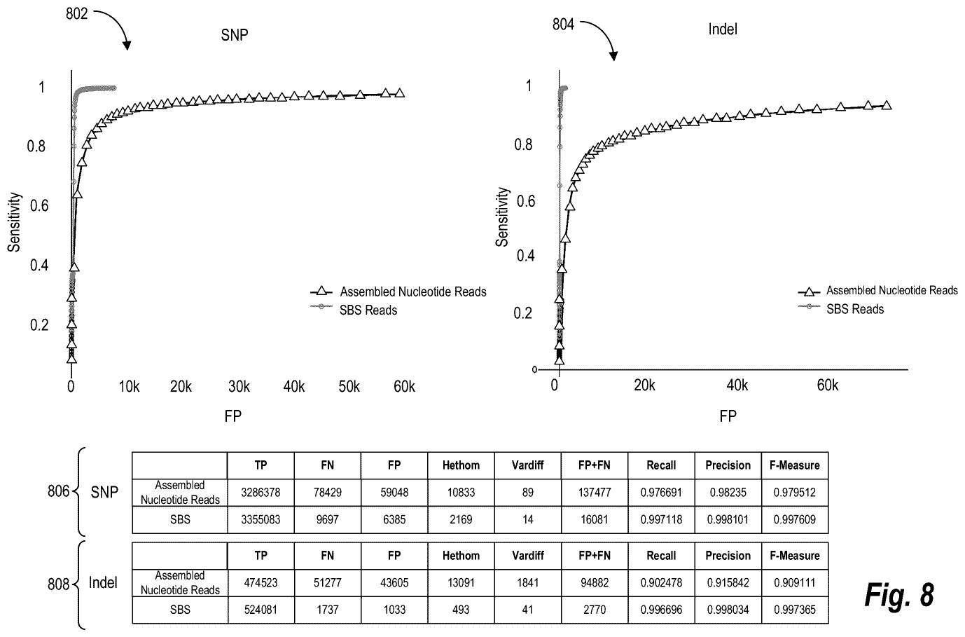

As mentioned above, in certain described embodiments, the call integration system 106 improves in accuracy over existing sequencing systems. In particular, the call integration system 106 reduces false positive variant genotype calls and false negative variant genotype calls compared to existing sequencing systems. Indeed, by utilizing a genotype-call-integration machine-learning model based on the described sequencing metrics, the call integration system 106 even improves over previous versions of the call generation model that did not utilize a genotype-call-integration machine-learning model (but which still outperform other systems). FIGS. 8-10B illustrate graphs and tables of experiments demonstrating the accuracy improvements of the call integration system 106.

For example, FIG. 8 illustrates performance of previous versions of a call generation model (e.g., a model that does not utilize a genotype-call-integration machine-learning model) in generating variant calls based on a PrecisionFDA dataset. For example, the previous version separately analyzes assembled nucleotide reads and SBS reads to generate independent results for SNPs and indels. The model generates variant calls for comparison with ground truth data (e.g., from a PrecisionFDA dataset, such as HG001 v4.2.1) to determine performance according to numbers of false positives and false negatives.

As shown, the graph 802 corresponds to the table 806, and the graph 804 corresponds to the table 808. The graph 802 depicts receiver operating characteristic (ROC) curves corresponding to the data of the table 806, where the previous version of the call generation model (e.g., without machine learning elements) determined variant calls for SNPs based on assembled nucleotide reads and SBS reads independently. Likewise, the graph 804 depicts ROC curves for the data of the table 808, where the previous version determined variant calls for indels based on assembled nucleotide reads and SBS reads independently. While the performance of the previous system is good in each case (e.g., without relatively few FPs and FNs compared to other prior systems), the call integration system 106 can nevertheless improve upon this performance by reducing false positives and/or false negatives.

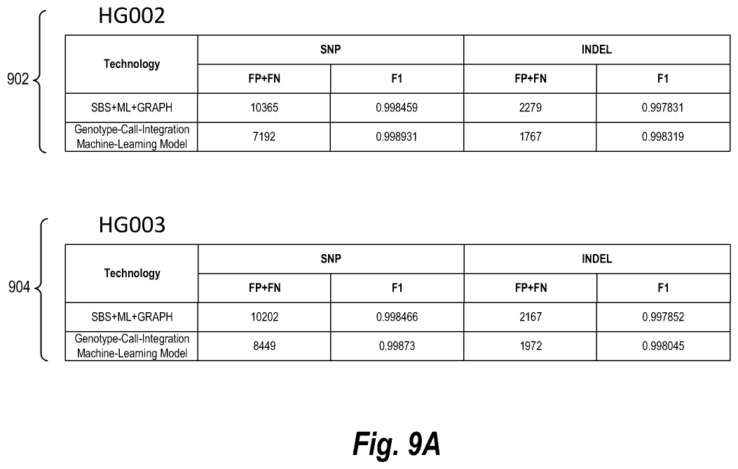

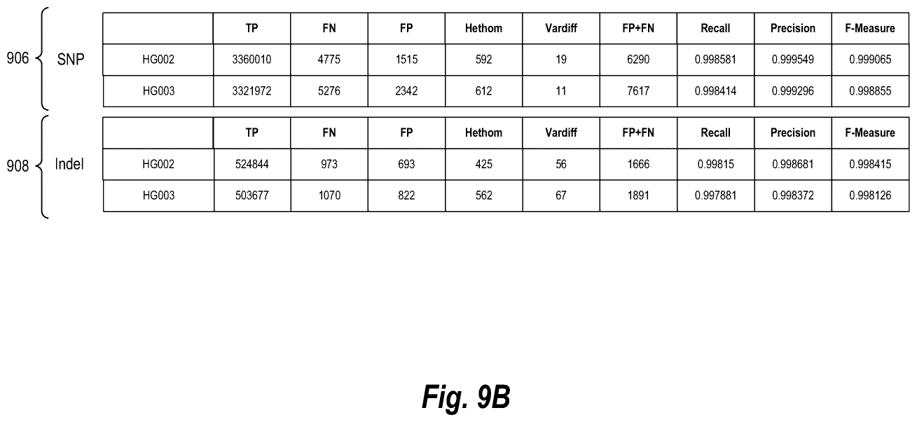

For instance, FIG. 9A illustrates tables comparing performance of a previous version of a call generation model with that of the call integration system 106. As shown, the table 902 depicts a cumulative indication of false positives and false negatives (FP+FN) for a variant calling model (SBS+ML+GRAPH) that uses single read types (e.g., SBS reads) together with machine learning predictions and a graph genome (e.g., the Illumina DRAGEN Graph Reference Genome hg19) to generate variant calls for SNPs and indels. The table 902 also depicts results from the call integration system 106 which utilizes the genotype-call-integration machine-learning model to generate variant calls (for SNPs and indels) based on both SBS reads and assembled nucleotide reads (in addition to using specific sequencing metrics and machine learning predictions).

As illustrated in FIG. 9A, experimenters generated the results of the table 902 by using the different models to generate variant calls for the HG002 dataset, which is a specific set of available human genome data for a certain genomic sample. In a similar fashion, the table 904 depicts results for both the previous model and the genotype-call-integration machine-learning model in generating variant calls for the HG003 dataset. As shown, the call integration system 106 with the genotype-call-integration machine-learning model outperforms the previous model, resulting in fewer FP+FN metrics in each table, with higher F1 scores in each case as well (e.g., for SNPs and indels in table 902 and in table 904). Indeed, by leveraging different read sources for different types of reads, the call integration system 106 can generate more accurate variant calls than systems that are not capable of processing multiple read types.

Continuing to FIG. 9B, the table 906 illustrates results generated by experimenters in using the genotype-call-integration machine-learning model to generate SNPs for the HG002 dataset and the HG003 dataset. In addition, the table 908 illustrates results generated by experimenters in using the genotype-call-integration machine-learning model to generate indels for the HG002 dataset and the HG003 dataset. Indeed, over longer training for the genotype-call-integration machine-learning model, experimenters have demonstrated further accuracy improvements beyond the metrics indicated in the previous figures. Compared to prior systems, the accuracy metrics of FIG. 9B (e.g., in table 906 and in table 908) indicate appreciable improvements in accuracy metrics for the genotype-call-integration machine-learning model, particularly in FN, FP, recall, precision, and F1-measure. Indeed, the accuracy metrics of the genotype-call-integration machine-learning model shown in FIG. 9B are improvements on those of the genotype-call-integration machine-learning model in FIG. 9A, which are still further improvements on prior systems that do not use the genotype-call-integration machine-learning model.

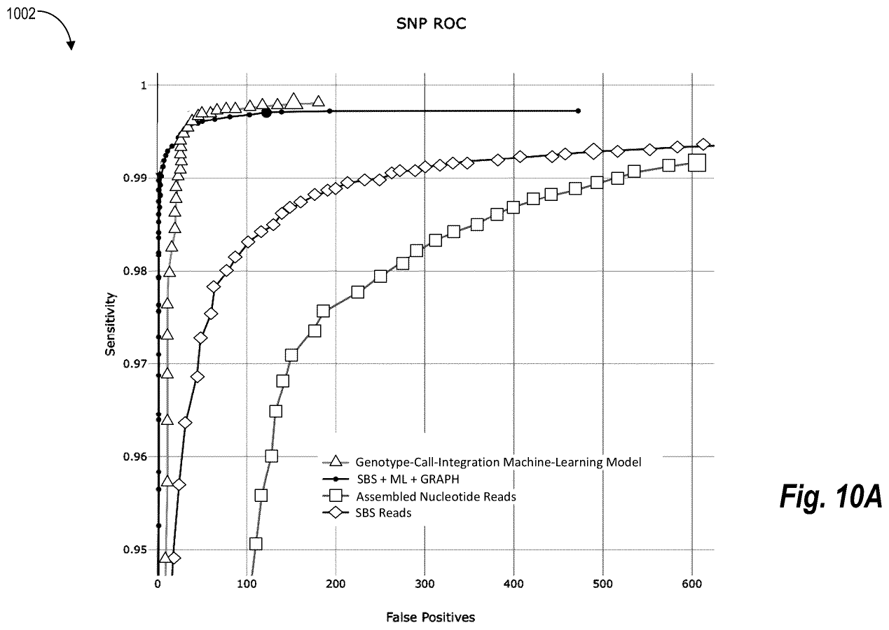

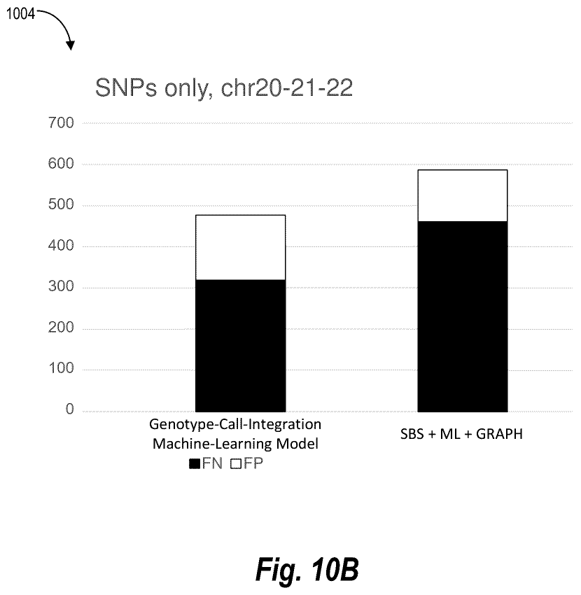

As illustrated in FIG. 10A, the graph 1002 depicts ROC curves for comparing the performance of different variant callers in generating variant calls for SNPs. For example, the graph 1002 illustrates ROC curves where the curves with the largest area under the curve generally perform best. As shown, the call integration system 106 with the genotype-call-integration machine-learning model outperforms the other models. The other models include the SBS+ML+GRAPH model (as reflected in the tables of FIG. 9), a model that generates variant calls (solely) from assembled nucleotide reads (e.g., without further analysis or machine learning techniques), and a model that generates variant calls (solely) from SBS reads (e.g., without further analysis or machine learning techniques). As indicated by the graph 1002, the genotype-call-integration machine-learning model has the highest area under the curve and the fewest false positives, outperforming the other models over the tested dataset (e.g., the PrecisionFDA dataset).

FIG. 10B illustrates a bar graph 1004 that coincides with the graph 1002. To elaborate, the bar graph 1004 provides an alternate visualization of the comparison between the genotype-call-integration machine-learning model and the SBS+ML+GRAPH model in variant calling for SNPs (e.g., for chromosomes 20-21-22). Indeed, the bar graph 1004 indicates false negatives and false positives, along with their cumulative totals for each model. As shown, the genotype-call-integration machine-learning model generates more accurate variant calls than the SBS+ML+GRAPH model, resulting in fewer false negatives, fewer false positives, and fewer FP+FN overall.

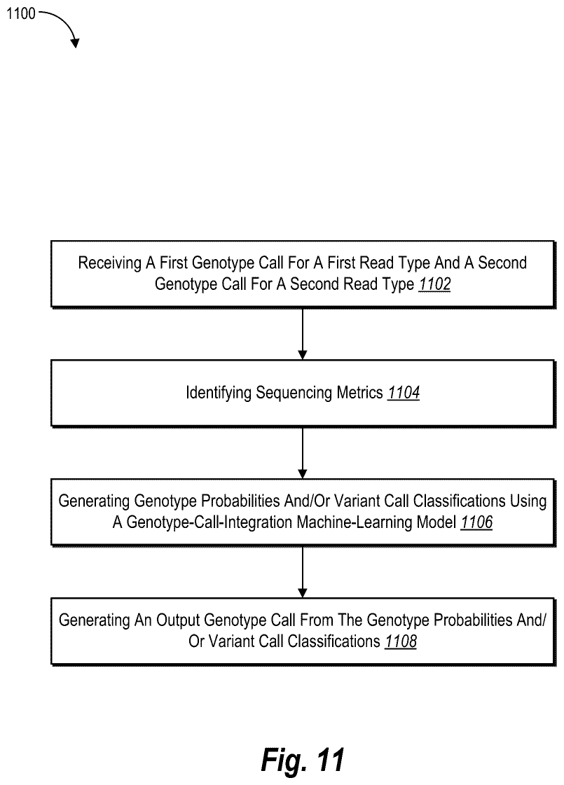

Turning now to FIG. 11, this figure illustrates an example flowchart of a series of acts of generating an output genotype call using a genotype-call-integration machine-learning model in accordance with one or more embodiments. While FIG. 11 illustrates acts according to one embodiment, alternative embodiments may omit, add to, reorder, and/or modify any of the acts shown in FIG. 11. The acts of FIG. 11 can be performed as part of a method. Alternatively, a non-transitory computer readable storage medium can comprise instructions that, when executed by one or more processors, cause a computing device to perform the acts depicted in FIG. 11. In still further embodiments, a system comprising at least one processor and a non-transitory computer readable medium comprising instructions that, when executed by one or more processors, cause the system to perform the acts of FIG. 11.

FIG. 11 illustrates a series of acts 1100 of generating an output genotype call using a genotype-call-integration machine-learning model. In particular, the series of acts 1100 includes an act 1102 of receiving a first genotype call for a first read type and a second genotype call for a second read type. For example, the act 1102 can involve receiving, for one or more genomic coordinates of a genomic sample, a first genotype call corresponding to a first type of nucleotide reads of a first threshold number of nucleobases and a second genotype call corresponding to a second type of nucleotide reads of a second threshold number of nucleobases. The first type of nucleotide reads can include nucleotide reads synthesized from sample library fragments that are shorter than the first threshold number of nucleobases. The second type of nucleotide reads can include assembled nucleotide reads that have been assembled from shorter nucleotide reads to form a contiguous sequence satisfying the first threshold number of nucleobases, circular consensus sequencing (CCS) reads satisfying the first threshold number of nucleobases, or nanopore long reads satisfying the first threshold number of nucleobases. The first genotype call can include a first variant call or a first reference call. The second genotype call can include a second variant call or a second reference call. In some cases, the first genotype call or the second genotype call include a null-data indicator

As further illustrated in FIG. 11, the series of acts 1100 can include an act 1104 of identifying sequencing metrics. In particular, the act 1104 can involve identifying sequencing metrics corresponding to the first genotype call or the second genotype call. For example, the act 1104 involves identifying the sequencing metrics corresponding to the first genotype call or the second genotype call by identifying one or more of a first set of sequencing metrics associated with the first genotype call corresponding to the first type of nucleotide reads, a second set of sequencing metrics associated with the second genotype call corresponding to the second type of nucleotide reads, or a shared set of sequencing metrics associated with both the first genotype call and the second genotype call. In some cases, the act 1104 involves identifying the sequencing metrics corresponding to the first genotype call or the second genotype call by determining one or more of read-based sequencing metrics, call-model-generated sequencing metrics, externally sourced sequencing metrics, or second-read-type sequencing metrics associated with the second genotype call corresponding to the second type of nucleotide reads.

In one or more embodiments, the act 1104 involves identifying read-based sequencing metrics comprising one or more of: an allele frequency corresponding to an allele for the first genotype call, an allele for the second genotype call, or a different allele for an alternative genotype call differing from the first and second genotype calls; a coverage depth of the first type of nucleotide reads corresponding to the first genotype call or the second type of nucleotide reads corresponding to the second genotype call; an average coverage depth of the first type of nucleotide reads corresponding to the first genotype call or the second type of nucleotide reads corresponding to the second genotype call; a mapping-quality metric for the first type of nucleotide reads corresponding to the first genotype call or the second type of nucleotide reads corresponding to the second genotype call; or a nucleobase composition of one or more nucleotide reads from the first type of nucleotide reads or the second type of nucleotide reads.

In certain embodiments, the act 1104 involves identifying call-model-generated sequencing metrics that include one or more of: a genotype metric, a base-call-quality metric, a genotype quality metric, a genotype probability metric, a genotype-likelihood metric (e.g., a non-PHRED-scaled-likelihood metric or a PHRED-scaled-likelihood metric) for the first genotype call determined from the first type of nucleotide reads or the second genotype call determined from the second type of nucleotide reads.

In these or other embodiments, the act 1104 involves identifying externally sourced sequencing metrics that include one or more of: a mappability metric indicating a degree of difficulty with which a nucleotide read is mapped to the one or more genomic coordinates within a reference genome; a guanine-cytosine-content metric indicating a count of guanine-cytosine content corresponding to the one or more genomic coordinates within the reference genome; a confidence classification or confidence score indicating a degree to which nucleobases at the one or more genomic coordinates can be accurately determined; a repeat classification indicating a category of repetitive genomic region for the one or more genomic coordinates; an indicator that the one or more genomic coordinates are part of a cytosine quadruplex (C-quadruplex) within the reference genome; an indicator that the one or more genomic coordinates are part of a guanine quadruplex (G-quadruplex) within the reference genome; or an indicator that the one or more genomic coordinates are part of a homopolymer within the reference genome.

Additionally, the series of acts 1100 can include an act 1106 of generating genotype probabilities and/or variant call classifications using a genotype-call-integration machine-learning model. In particular, the act 1106 can involve generating, utilizing a genotype-call-integration machine-learning model and based on the sequencing metrics, genotype probabilities of genotype calls for the one or more genomic coordinates. In some cases, the act 1106 involves generating, utilizing a genotype-call-integration machine-learning model and based on the sequencing metrics, variant call classifications for candidate variant calls at the one or more genomic coordinates.

In one or more embodiments, the act 1106 involves generating the genotype probabilities by generating the genotype probabilities for one or more candidate single nucleotide polymorphisms (SNPs) utilizing the genotype-call-integration machine-learning model trained with SNP training data. In certain embodiments, the act 1106 involves generating a first genotype probability of the genomic sample comprising a homozygous reference genotype at the one or more genomic coordinates, generating a second genotype probability of the genomic sample comprising a heterozygous variant genotype at the one or more genomic coordinates, and generating a third genotype probability of the genomic sample comprising a homozygous variant genotype at the one or more genomic coordinates.

In certain embodiments, the act 1106 involves generating the variant call classifications for one or more candidate insertions or deletions (indels) utilizing the genotype-call-integration machine-learning model trained with indel training data. The act 1106 can involve generating the variant call classifications for the candidate variant calls by generating one or more of: a first true-positive variant probability that the first genotype call constitutes a true positive variant for the one or more genomic coordinates; a second true-positive variant probability that the second genotype call constitutes a true positive variant for the one or more genomic coordinates; a first zygosity-error probability that the first genotype call comprises a genotype-zygosity error at the one or more genomic coordinates; a second zygosity-error probability that the second genotype call comprises a genotype-zygosity error at the one or more genomic coordinates; or a reference probability of a homozygous reference genotype at the one or more genomic coordinates.

In some embodiments, the series of acts 1108 includes an act 1108 of generating an output genotype call from the genotype probabilities and/or the variant call classifications. In particular, the act 1108 can involve generating an output genotype call for the one or more genomic coordinates of the genomic sample based on the genotype probabilities. In some cases, the act 1108 involves generating an output genotype call for the one or more genomic coordinates of the genomic sample based on the variant call classifications. In certain embodiments, the act 1108 involves generating the output genotype call indicating a presence or absence of an SNP at the one or more genomic coordinates of the genomic sample. In some embodiments, the act 1108 involves generating the output genotype call indicating a presence or absence of an indel at the one or more genomic coordinates of the genomic sample. The act 1108 can include selecting the first genotype call or the second genotype call or generating a different genotype call differing from the first genotype call and the second genotype call.

In certain embodiments, the act 1108 involves selecting the first genotype call instead of the second genotype call. Selecting the first genotype call instead of the second genotype call can involve selecting a homozygous reference genotype call from the first genotype call instead of a heterozygous variant genotype call or a homozygous variant genotype call from the second genotype call, selecting the heterozygous variant genotype call from the first genotype call instead of the homozygous reference genotype call or the homozygous variant genotype call from the second genotype call, or selecting the homozygous variant genotype call from the first genotype call instead of the heterozygous variant genotype call or the homozygous reference genotype call from the second genotype call.

In some cases, the act 1108 involves selecting the second genotype call instead of the first genotype call by selecting a homozygous reference genotype call from the second genotype call instead of a heterozygous variant genotype call or a homozygous variant genotype call from the first genotype call, selecting the heterozygous variant genotype call from the second genotype call instead of the homozygous reference genotype call or the homozygous variant genotype call from the first genotype call, or selecting the homozygous variant genotype call from the second genotype call instead of the heterozygous variant genotype call or the homozygous reference genotype call from the first genotype call. The act 1108 can involve selecting the first genotype call or the second genotype call or generating a different genotype call differing from the first genotype call and the second genotype call.

In one or more embodiments, the series of acts 1100 includes an act of modifying a genotype metric, a base-call-quality metric, a genotype quality metric, a genotype probability metric, a genotype-likelihood metric, or a PHRED-scaled-genotype-likelihood metric based on the genotype probabilities and/or the variant call classifications. In these or other embodiments, the series of acts 1100 includes an act of generating a variant call file that includes the modified genotype metric, the modified base-call-quality metric, the modified genotype quality metric, the modified genotype probability metric, the modified genotype-likelihood metric, or the modified PHRED-scaled-genotype-likelihood metric.

In certain embodiments, the series of acts 1100 includes an act of receiving the first genotype call by receiving the first genotype call as part of a first variant call file based on the first type of nucleotide reads. In the same or other embodiments, the series of acts 1100 includes acts of receiving the second genotype call by receiving the second genotype call as part of a second variant call file based on the second type of nucleotide reads and generating a merged variant call file comprising the first genotype call or the second genotype call.

In some embodiments, the series of acts 1100 includes an act of determining that the first genotype call comprises a first alternate nucleobase that differs from a second alternate nucleobase of the second genotype call. The series of acts 1100 can also include an act of generating, utilizing the genotype-call-integration machine-learning model and based on the sequencing metrics, a first pipeline-accuracy likelihood of the first genotype call being more accurate than the second genotype call and a second pipeline-accuracy likelihood of the second genotype call being more accurate than the first genotype call. Further, the series of acts 1100 can include an act of generating the output genotype call by selecting the first genotype call or the second genotype call for the one or more genomic coordinates of the genomic sample based on the first pipeline-accuracy likelihood and the second pipeline-accuracy likelihood.

The series of acts 1100 can include an act of determining that the first true-positive variant probability fails to satisfy a likelihood threshold. In addition, the series of acts 1100 can include an act 1100 of, based on determining that the first true-positive variant probability fails to satisfy the likelihood threshold, generating or utilizing the second true-positive variant probability.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleobase type from another are particularly applicable. In some embodiments, the process to determine the nucleotide sequence of a target nucleic acid (i.e., a nucleic acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using 7-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (Ppi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released Ppi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and 7-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the call integration system 106 can include software, hardware, or both. For example, the components of the call integration system 106 can include one or more instructions stored on a computer-readable storage medium and executable by processors of one or more computing devices (e.g., the client device 108). When executed by the one or more processors, the computer-executable instructions of the call integration system 106 can cause the computing devices to perform the bubble detection methods described herein. Alternatively, the components of the call integration system 106 can comprise hardware, such as special purpose processing devices to perform a certain function or group of functions. Additionally, or alternatively, the components of the call integration system 106 can include a combination of computer-executable instructions and hardware.

Furthermore, the components of the call integration system 106 performing the functions described herein with respect to the call integration system 106 may, for example, be implemented as part of a stand-alone application, as a module of an application, as a plug-in for applications, as a library function or functions that may be called by other applications, and/or as a cloud-computing model. Thus, components of the call integration system 106 may be implemented as part of a stand-alone application on a personal computing device or a mobile device. Additionally, or alternatively, the components of the call integration system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 12 illustrates a block diagram of a computing device 1200 that may be configured to perform one or more of the processes described above. One will appreciate that one or more computing devices such as the computing device 1200 may implement the call integration system 106 and the sequencing system 104. As shown by FIG. 12, the computing device 1200 can comprise a processor 1202, a memory 1204, a storage device 1206, an I/O interface 1208, and a communication interface 1210, which may be communicatively coupled by way of a communication infrastructure 1212. In certain embodiments, the computing device 1200 can include fewer or more components than those shown in FIG. 12. The following paragraphs describe components of the computing device 1200 shown in FIG. 12 in additional detail.

In one or more embodiments, the processor 1202 includes hardware for executing instructions, such as those making up a computer program. As an example, and not by way of limitation, to execute instructions for dynamically modifying workflows, the processor 1202 may retrieve (or fetch) the instructions from an internal register, an internal cache, the memory 1204, or the storage device 1206 and decode and execute them. The memory 1204 may be a volatile or non-volatile memory used for storing data, metadata, and programs for execution by the processor(s). The storage device 1206 includes storage, such as a hard disk, flash disk drive, or other digital storage device, for storing data or instructions for performing the methods described herein.

The I/O interface 1208 allows a user to provide input to, receive output from, and otherwise transfer data to and receive data from computing device 1200. The I/O interface 1208 may include a mouse, a keypad or a keyboard, a touch screen, a camera, an optical scanner, network interface, modem, other known I/O devices or a combination of such I/O interfaces. The I/O interface 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1208 is configured to provide graphical data to a display for presentation to a user. The graphical data may be representative of one or more graphical user interfaces and/or any other graphical content as may serve a particular implementation.

The communication interface 1210 can include hardware, software, or both. In any event, the communication interface 1210 can provide one or more interfaces for communication (such as, for example, packet-based communication) between the computing device 1200 and one or more other computing devices or networks. As an example, and not by way of limitation, the communication interface 1210 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1210 may facilitate communications with various types of wired or wireless networks. The communication interface 1210 may also facilitate communications using various communication protocols. The communication infrastructure 1212 may also include hardware, software, or both that couples components of the computing device 1200 to each other. For example, the communication interface 1210 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer readable medium storing instructions that, when executed by the at least one processor, cause the system to:
receive, for one or more genomic coordinates of a genomic sample, a first genotype call corresponding to a first type of nucleotide reads of a first threshold number of nucleobases and a second genotype call corresponding to a second type of nucleotide reads of a second threshold number of nucleobases;
identify sequencing metrics corresponding to the first genotype call or the second genotype call;
generate, utilizing a genotype-call-integration machine-learning model and based on the sequencing metrics, genotype probabilities of genotype calls for the one or more genomic coordinates; and
generate an output genotype call for the one or more genomic coordinates of the genomic sample based on the genotype probabilities.

2. The system of claim 1, wherein:
the first type of nucleotide reads comprise nucleotide reads synthesized from sample library fragments that are shorter than the first threshold number of nucleobases; and
the second type of nucleotide reads comprises:
assembled nucleotide reads that have been assembled from shorter nucleotide reads to form a contiguous sequence satisfying the first threshold number of nucleobases;
circular consensus sequencing (CCS) reads satisfying the first threshold number of nucleobases; or
nanopore long reads satisfying the first threshold number of nucleobases.

3. The system of claim 1, further storing instructions that, when executed by the at least one processor, cause the system to:
generate the genotype probabilities by generating the genotype probabilities for one or more candidate single nucleotide polymorphisms (SNPs) utilizing the genotype-call-integration machine-learning model trained with SNP training data; and
generate the output genotype call indicating a presence or absence of an SNP at the one or more genomic coordinates of the genomic sample.

4. The system of claim 1, further storing instructions that, when executed by the at least one processor, cause the system to generate the output genotype call by:
selecting the first genotype call or the second genotype call; or
generating a different genotype call differing from the first genotype call and the second genotype call.

5. The system of claim 1, further storing instructions that, when executed by the at least one processor, cause the system to generate the genotype probabilities by:
generating a first genotype probability of the genomic sample comprising a homozygous reference genotype at the one or more genomic coordinates;
generating a second genotype probability of the genomic sample comprising a heterozygous variant genotype at the one or more genomic coordinates; and
generating a third genotype probability of the genomic sample comprising a homozygous variant genotype at the one or more genomic coordinates.

6. The system of claim 1, wherein the first genotype call comprises a first variant call or a first reference call, and the second genotype call comprises a second variant call or a second reference call.

7. The system of claim 1, wherein the first genotype call or the second genotype call comprises a null-data indicator.

8. The system of claim 1, further storing instructions that, when executed by the at least one processor, cause the system to:
modify a genotype metric, a base-call-quality metric, a genotype quality metric, a genotype probability metric, a genotype-likelihood metric, or a PHRED-scaled-genotype-likelihood metric based on the genotype probabilities; and
generate a variant call file that includes the modified genotype metric, the modified base-call-quality metric, the modified genotype quality metric, the modified genotype probability metric, the modified genotype-likelihood metric, or the modified PHRED-scaled-genotype-likelihood metric.

9. The system of claim 1, further storing instructions that, when executed by the at least one processor, cause the system to generate the output genotype call by selecting the first genotype call instead of the second genotype call by:
selecting a homozygous reference genotype call from the first genotype call instead of a heterozygous variant genotype call or a homozygous variant genotype call from the second genotype call;
selecting the heterozygous variant genotype call from the first genotype call instead of the homozygous reference genotype call or the homozygous variant genotype call from the second genotype call; or
selecting the homozygous variant genotype call from the first genotype call instead of the heterozygous variant genotype call or the homozygous reference genotype call from the second genotype call.

10. The system of claim 1, further storing instructions that, when executed by the at least one processor, cause the system to generate the output genotype call by selecting the second genotype call instead of the first genotype call by:
selecting a homozygous reference genotype call from the second genotype call instead of a heterozygous variant genotype call or a homozygous variant genotype call from the first genotype call;
selecting the heterozygous variant genotype call from the second genotype call instead of the homozygous reference genotype call or the homozygous variant genotype call from the first genotype call; or
selecting the homozygous variant genotype call from the second genotype call instead of the heterozygous variant genotype call or the homozygous reference genotype call from the first genotype call.

11. A non-transitory computer readable medium storing instructions that, when executed by at least one processor, cause a system to:
receive, for one or more genomic coordinates of a genomic sample, a first genotype call corresponding to a first type of nucleotide reads of a first threshold number of nucleobases and a second genotype call corresponding to a second type of nucleotide reads of a second threshold number of nucleobases;
identify sequencing metrics corresponding to the first genotype call or the second genotype call;
generate, utilizing a genotype-call-integration machine-learning model and based on the sequencing metrics, genotype probabilities of genotype calls for the one or more genomic coordinates; and
generate an output genotype call for the one or more genomic coordinates of the genomic sample based on the genotype probabilities.

12. The non-transitory computer readable medium of claim 11, further storing instructions that, when executed by the at least one processor, cause the system to identify the sequencing metrics corresponding to the first genotype call or the second genotype call by identifying one or more of:
a first set of sequencing metrics associated with the first genotype call corresponding to the first type of nucleotide reads;
a second set of sequencing metrics associated with the second genotype call corresponding to the second type of nucleotide reads; or
a shared set of sequencing metrics associated with both the first genotype call and the second genotype call.

13. The non-transitory computer readable medium of claim 11, further storing instructions that, when executed by the at least one processor, cause the system to identify the sequencing metrics corresponding to the first genotype call or the second genotype call by determining one or more of read-based sequencing metrics, call-model-generated sequencing metrics, externally sourced sequencing metrics, or second-read-type sequencing metrics associated with the second genotype call corresponding to the second type of nucleotide reads.

14. The non-transitory computer readable medium of claim 11, further storing instructions that, when executed by the at least one processor, cause the system to identify the sequencing metrics corresponding to the first genotype call or the second genotype call by identifying read-based sequencing metrics comprising one or more of:
an allele frequency corresponding to an allele for the first genotype call, an allele for the second genotype call, or a different allele for an alternative genotype call differing from the first and second genotype calls;
a coverage depth of the first type of nucleotide reads corresponding to the first genotype call or the second type of nucleotide reads corresponding to the second genotype call;
an average coverage depth of the first type of nucleotide reads corresponding to the first genotype call or the second type of nucleotide reads corresponding to the second genotype call;
a mapping-quality metric for the first type of nucleotide reads corresponding to the first genotype call or the second type of nucleotide reads corresponding to the second genotype call; or
a nucleobase composition of one or more nucleotide reads from the first type of nucleotide reads or the second type of nucleotide reads.

15. The non-transitory computer readable medium of claim 11, further storing instructions that, when executed by the at least one processor, cause the system to identify the sequencing metrics corresponding to the first genotype call or the second genotype call by identifying call-model-generated sequencing metrics comprising one or more of: a genotype metric, a base-call-quality metric, a genotype quality metric, a genotype probability metric, or a PHRED-scaled-likelihood metric for the first genotype call determined from the first type of nucleotide reads or the second genotype call determined from the second type of nucleotide reads.

16. The non-transitory computer readable medium of claim 11, further storing instructions that, when executed by the at least one processor, cause the system to identify the sequencing metrics corresponding to the first genotype call or the second genotype call by identifying externally sourced sequencing metrics comprising one or more of:
a mappability metric indicating a degree of difficulty with which a nucleotide read is mapped to the one or more genomic coordinates within a reference genome;
a guanine-cytosine-content metric indicating a count of guanine-cytosine content corresponding to the one or more genomic coordinates within the reference genome;
a confidence classification or confidence score indicating a degree to which nucleobases at the one or more genomic coordinates can be accurately determined;
a repeat classification indicating a category of repetitive genomic region for the one or more genomic coordinates;
an indicator that the one or more genomic coordinates are part of a cytosine quadruplex (C-quadruplex) within the reference genome;
an indicator that the one or more genomic coordinates are part of a guanine quadruplex (G-quadruplex) within the reference genome; or
an indicator that the one or more genomic coordinates are part of a homopolymer within the reference genome.

17. A computer-implemented method comprising:
receiving, for one or more genomic coordinates of a genomic sample, a first genotype call corresponding to a first type of nucleotide reads of a first threshold number of nucleobases and a second genotype call corresponding to a second type of nucleotide reads of a second threshold number of nucleobases;
identifying sequencing metrics corresponding to the first genotype call or the second genotype call;
generating, utilizing a genotype-call-integration machine-learning model and based on the sequencing metrics, genotype probabilities of genotype calls for the one or more genomic coordinates; and
generating an output genotype call for the one or more genomic coordinates of the genomic sample based on the genotype probabilities.

18. The computer-implemented method of claim 17, wherein:
the first type of nucleotide reads comprise nucleotide reads synthesized from sample library fragments that are shorter than the first threshold number of nucleobases; and
the second type of nucleotide reads comprises:
assembled nucleotide reads that have been assembled from shorter nucleotide reads to form a contiguous sequence satisfying the first threshold number of nucleobases;
circular consensus sequencing (CCS) reads satisfying the first threshold number of nucleobases; or
nanopore long reads satisfying the first threshold number of nucleobases.

19. The computer-implemented method of claim 17, further comprising:
receiving the first genotype call by receiving the first genotype call as part of a first variant call file based on the first type of nucleotide reads;
receiving the second genotype call by receiving the second genotype call as part of a second variant call file based on the second type of nucleotide reads; and
generating a merged variant call file comprising the first genotype call or the second genotype call.

20. The computer-implemented method of claim 17, further comprising:
determining that the first genotype call comprises a first alternate nucleobase that differs from a second alternate nucleobase of the second genotype call;
generating, utilizing the genotype-call-integration machine-learning model and based on the sequencing metrics, a first pipeline-accuracy likelihood of the first genotype call being more accurate than the second genotype call and a second pipeline-accuracy likelihood of the second genotype call being more accurate than the first genotype call; and
generating the output genotype call by selecting the first genotype call or the second genotype call for the one or more genomic coordinates of the genomic sample based on the first pipeline-accuracy likelihood and the second pipeline-accuracy likelihood.
